(19) United States
(12) Patent Application Publication
(10) Pub. No.: US 2003/0175906 A1
(43) Pub. Date: Sep. 18, 2003

(54) Nuclease resistant chimeric oligonucleotides

(76) Inventors: Muthiah Manoharan, Carlsbad, CA (US); Martin A. Maier, Carlsbad, CA (US); Thazha P. Prakash, Carlsbad, CA (US); Kallanthottathil G. Rajeev, Solana Beach, CA (US)

Correspondence Address: Joseph Lucci, WOODCOCK WASHBURN LLP, 46th Floor, One Liberty Place, Philadelphia, PA, 19103, US

(21) Appl. No.: 10/013,295

(22) Filed: Dec. 10, 2001

Related U.S. Application Data
(60) Provisional application No. 60/302,682, filed on Jul. 3, 2001.

(51) Int. Cl. C12P 19/34; C07H 21/04

(52) U.S. Cl. 435/91.2; 536/23.1

(57) ABSTRACT

The present invention relates to novel nuclease-resistant oligomeric compounds and to novel methods for increasing the nuclease resistance of oligomeric compounds. In preferred embodiments of the invention, the oligomeric compounds comprise at least one modified nucleoside containing a modified sugar moiety at either the 3′ or 5′ terminus of the oligomeric compound, and farther comprise at least one internucleoside linking group that is other than phosphodiester. Other preferred embodiments of the invention include methods of enhancing the nuclease resistance of oligomeric compounds comprising incorporating at least one modified nucleoside containing a modified sugar moiety at either the 3′ or 5′ terminus of an oligomeric compound.

### CROSS REFERENCE TO RELATED APPLICATIONS

This application claims the benefit of U.S. Provisional Application Ser. No. 60/302,682, filed Jul. 3, 2001.

### FIELD OF THE INVENTION

The present invention relates to novel nuclease-resistant oligomeric compounds and to novel methods for increasing the nuclease resistance of oligomeric compounds.

### BACKGROUND OF THE INVENTION

Efficacy and sequence specific behavior of antisense oligonucleotides (ONs) in biological systems depend upon their resistance to enzymatic degradation. It is therefore essential, when designing potent antisense drugs, to combine features such as high binding affinity and mismatch sensitivity with nuclease resistance. Unmodified phosphodiester antisense oligonucleotides are degraded rapidly in biological fluids containing hydrolytic enzymes (Shaw, J. P.; Kent, K.; Bird, J.; Fishback, J.; Froehler, B. Nucleic Acids Res. 1991, 19, 747-750;Woolf, T. M.; Jennings, C. G. B.; Rebagliati, M; Melton, D. A. Nucleic Acids Res. 1990, 18, 1763-1769), and the first generation of modified antisense oligonucleotide drugs, such as 2′-deoxyphosphorothioate oligonucleotides, were also subject to enzymatic degradation (Maier, M.; Bleicher, K.; Kalthoff, H.; Bayer, E. Biomed. Pept., Proteins Nucleic Acids 1995, 1, 235-241; Agrawal, S.; Temsamani, J.; Tang, J. Y. Proc. Natl. Acad. Sci. 1991, 88, 7595-7599). Extensive stability against the various nucleases present in biological systems can best be achieved by modified oligonucleotides. Since 3′ exonuclease activity is predominantly responsible for enzymatic degradation in serum-containing medium and in various eukaryotic cell lines, modifications located at the 3′-terminus significantly contribute to the nuclease resistance of an oligonucleotide (Shaw, J. -P.; Kent, K.; Bird, J.; Fishback, J.; Froehler, B. Nucleic Acids Res. 1991, 19, 747-750; Maier, M.; Bleicher, K.; Kalthoff, H.; Bayer, E. Biomed. Pept., Proteins Nucleic Acids 1995, 1, 235-241).

Extensive modifications have been made to the phosphodiester linkages and sugar moieties of oligonucleotides, while modifications to the heterocyclic base moieties have been relatively limited, due to a desire to maintain the specific hydrogen bonding motifs required for base pair specificity (For a review see, Herdewijn, P. Antisense Nucleic Acids Drug Dev. 2000, 10, 297-310). The 2′-position is attractive for derivatization because it offers the advantages of enhancing both nuclease resistance and binding affinity (Manoharan, M. Biochim. Biophys. Acta 1999, 1489, 117-130; Kawasaki, A. M.; Casper, M. D.; Prakash, T. P.; Manalili, S.; Sasmor, H.; Manoharan, M.; Cook, P. D. Nucleosides Nucleotides 1999, 18, 1419-1420).

A large number of nucleobase modifications, which were designed to enhance the binding affinity of antisense oligonucleotides to their complementary target strands, have recently been introduced (Beaucage, S. L.; Iyer, R. P. Tetrahedron 1993; 49, 6123-94; Cook, P. D. Annu. Rep. Med. Chem. 1998, 33, 313-325; Goodchild, J. Bioconjugate Chemistry, 1990; 1, 165-87; Uhlmann, E.; Peyman, A. Chem. Rev. 1990, 90, 543-84. For reviews see: Uhlmann, E.; Peyman, A. Chem. Rev. 1990, 90, 543-584; Milligan, J. F.; Matteucci, M. D.; Martin, J. C. J. Med. Chem. 1993, 36, 1923-37; Cook, P. D. Antisense medicinal chemistry. In: Antisense Research and Application, A Handbook of Experimental Pharmacology (ed. Crooke, S. T.), pp. 51-101. Springer-Verlag, New York, 1998). Some heterocyclic modifications have been shown to enhance the binding affinity of nucleic acids through increased hydrogen bonding and/or base stacking interactions. Examples of such heterocyclic modifications include 2,6-diaminopurine, which allows for a third hydrogen bond with thymidine and replacement of the hydrogen atom at the C5 position of pyrimidine bases with a propynyl group, resulting in increased stacking interactions (Chollet, A.; Chollet-Damerius, A.; Kawashima, E. H. Chem. Scripta 1986, 26, 37-40; Wagner, R. W.; Matteucci, M. D.; Lewis, J. G.; Guttierrez, A. J.; Moulds, C.; Froehler, B. C. Science 1993,260, 1510-1513).

More recently, several tricyclic cytosine analogs, such as phenoxazine, phenothiazine (Lin, K. -Y.; Jones, R. J.; Matteucci, M. J. Am. Chem. Soc. 1995, 117, 3873-3874) and tetrafluorophenoxazin (Wang, J.; Lin, K. -Y., Matteucci, M. Tetrahedron Lett. 1998, 39, 8385-8388), have been developed and have been shown to hybridize to guanine and, in case of tetrafluorophenoxazin, also with adenine. The tricyclic cytosine analogs have also been shown to enhance helical thermal stability by extended stacking interactions.

The helix-stabilizing properties of the tricyclic cytosine analogs are further improved with G-clamp, a cytosine analog with an aminoethoxy moiety attached to the rigid phenoxazine scaffold (Lin, K. -Y.; Matteucci, M. J. Am. Chem. Soc. 1998, 120, 8531-8532). Binding studies have demonstrated that a single G-clamp enhances the binding affinity of a model oligonucleotide to its complementary target DNA or RNA with a ΔTₘ of up to 18° relative to 5-methyl cytosine (dC5^{me}), the highest known affinity enhancement for a single modification. The gain in helical stability does not compromise the binding specificity of the oligonucleotides, as the Tₘ data indicate an even greater discrimination between the perfectly matched and mismatched sequences as compared to dC5^{me}. The tethered amino group may serve as an additional hydrogen bond donor that interacts with the Hoogsteen face, namely the O6, of a complementary guanine. The increased affinity of G-clamp is thus most likely mediated by the combination of extended base stacking and additional hydrogen bonding.

The enhanced binding affinity of the phenoxazine derivatives together with their uncompromised sequence specificity makes them valuable nucleobase analogs for the development of more potent antisense-based drugs. Promising data have been derived from in vitro experiments demonstrating that heptanucleotides containing phenoxazine substitutions are capable of activating RNaseH, enhance cellular uptake, and exhibit an increased antisense activity (Lin, K. -Y.; Matteucci, M. J. Am. Chem. Soc. 1998, 120, 8531-8532). The activity enhancement was even more pronounced in the case of G-clamp, as a single substitution was shown to significantly improve the in vitro potency of a 20 mer 2′-deoxyphosphorothioate oligonucleotide (Flanagan, W. M.; Wolf, J. J.; Olson, P.; Grant, D.; Lin, K. -Y.; Wagner, R. W.; Matteucci, M. Proc. Natl. Acad. Sci. USA, 1999, 96, 3513-3518).

The efficacy and sequence specificicy of oligonucleotides in biological systems is dependent, in part, upon their nuclease stability. Resistance to the many nucleases present in biological systems is best achieved by modified oligonucleotides. It is therefore essential, when designing modified nucleotides, to evaluate and optimize their resistance to enzymatic degradation.

### SUMMARY OF THE INVENTION

The present invention relates to novel nuclease-resistant oligomeric compounds and to novel methods for increasing the nuclease resistance of oligomeric compounds.

In preferred embodiments, the compounds of the invention relate to oligomeric compounds of formula V:

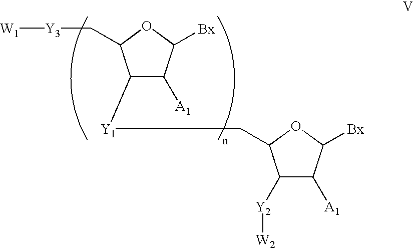

wherein:

n is from 3 to about 50;

each Y₁ is, independently, an internucleoside linking group;

Y₂ is oxygen or an internucleoside linking group;

Y₃ is oxygen or an internucleoside linking group;

each Bx is an optionally protected heterocyclic base moiety;

each A₁ is, independently, hydrogen or a sugar substituent group;

W₁ is hydrogen, a hydroxyl protecting group or a modified nucleoside selected from the group consisting of

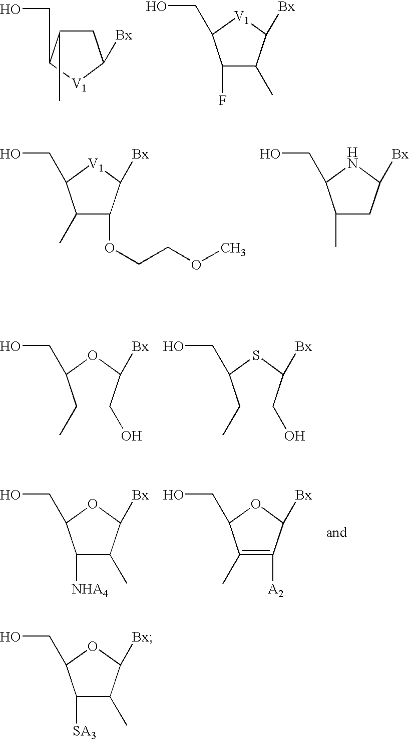

W₂ is hydrogen, a hydroxyl protecting group or a modified nucleoside selected from the group consisting of

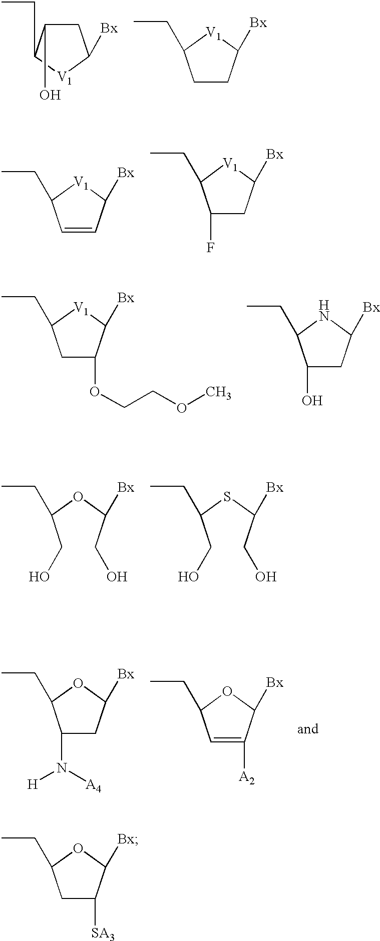

each A₂ is, independently, alkyl, alkenyl, alkynyl, aryl, alkaryl, O-alkyl, O-aryl, amino, substituted amino, —SH, —SA₃, thiolether, F, or morpholino;

each A₃ is, independently, H, a sulfur protecting group, aryl, alkaryl, substituted or unsubstituted C₁-C₁₀ alkyl, substituted or unsubstituted C₂-C₁₀ alkenyl, substituted or unsubstituted C₂-C₁₀ alkynyl, or alkaryl, wherein said substitution is OA₅ or SA₅;

each A₄ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, or alkaryl, wherein said substitution is OA₅ or SA₅;

each A₅ is, independently, hydrogen, C₁-C₁₀ alkyl, cycloalkyl or aryl;

each V₁ is, independently, O or S;

wherein at least one of W₁ and W₂ is not hydrogen or a hydroxyl protecting group and at least one internucleoside linking group is not a phosphodiester linking group.

In certain preferred embodiments, the internucleoside linking groups of the compounds of formula V are phosphorus-containing internucleoside linking groups. In still more preferred embodiments, at least one internucleoside linking group of the compounds of formula V is other than phosphodiester, and more preferably, greater than 90% of the internucleoside linking groups of the compounds of formula V are non-phosphorous containing internucleoside linking groups. In even more preferred embodiments, greater than 90% of the internucleoside linking group of the compounds of formula V are phosphorothioate linking groups.

In certain other embodiments of the invention, the oligomeric compounds of formula V comprise gapmers, hemimers or inverted gapmers. In more preferred embodiments, the oligomeric compounds of formula V comprise at least one 2′—O—CH₂CH₂—O—CH₃ sugar substituent group in at least one region of the gapmer, hemimer of inverted gapmer.

In other embodiments of the invention, the oligomeric compounds of formula V comprise at least one nucleoside wherein Bx is a polycyclic heterocyclic base moiety. In more preferred embodiments, the oligomeric compounds of formula V comprise at least one nucleoside wherein Bx is, independently, of the formula:

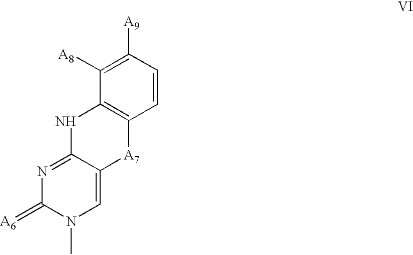

wherein

A₆ is O or S;

A₇ is CH₂, N—CH₃, O or S;

each A₈ and A₉ is hydrogen or one of A₈ and A₉ is hydrogen and the other of A₈ and A₉ is selected from the group consisting of:

—O—(CH₂)ₚ₁—G

and

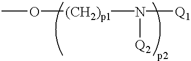

wherein

G is —CN, —OA₁₀, —SA₁₀, —N(H)A₁₀, —ON(H)A₁₀ or —C(═NH)N(H)A₁₀;

Q₁ is H, —NHA₁₀, —C(═O)N(H)A₁₀, —C(═S)N(H)A₁₀ or —C(═NH)N(H)A₁₀,

each Q₂ is, independently, H or Pg;

A₁₀ is H, Pg, substituted or unsubstituted C₁-C₁₀ alkyl, acetyl, benzyl, —(CH₂)ₚ₃NH₂, —(CH₂)ₚ₃N(H)Pg, a D or L α-amino acid, or a peptide derived from D, L or racemic α-amino acids;

Pg is a nitrogen, oxygen or thiol protecting group;

each p1 is, independently, from 2 to about 6;

p2 is from 1 to about 3; and

p3 is from 1 to about 4.

In another embodiment of the invention, Y₃ of formula V is an internucleoside likning group and W₁ of formula V is a modified nucleoside. In another embodiment of the invention, Y₂ of formula V is an internucleoside linking group and W₂ of formula V is a modified nucleoside.

In certain preferred embodiments of the invention, each sugar substituent group of formula V is, independently, —O—CH₂CH₂OCH₃, —O(CH₂)₂ON(CH₃)₂, —O—(CH₂)₂—O—(CH₂)₂—N(CH₃)₂, —O—CH₃, —OCH₂CH₂CH₂NH₂, —CH₂—CH═CH₂, or fluoro.

In another preferred embodiment, the invention relates to methods of enhancing the nuclease resistance of an oligomeric compound comprising providing at least one modified nucleoside at either the 3′ or 5′ terminus of the oligomeric compound to give a modified oligomeric compound of formula V, such that at least one of W₁ and W₂ of formula V is not hydrogen or a hydorxyl protecting group.

### BRIEF DESCRIPTION OF THE DRAWINGS

FIG. 1 depicts the structure of the tricyclic cytosine analog G-clamp, its extended analog guanidino G-clamp hybridized to complementary guanosine, and a palindromic decamer duplex that was used for x-ray crystallography.

FIG. 2 depicts a Fourier sum electron density map of a guanidino G-clamp nucleoside analog hybridized to guanosine.

FIG. 3 depicts the base stacking that occurs between a guanidinyl G-clamp nucleobase analog and guanine viewed approximately along the vertical to the phenoxazine rings.

FIG. 4 depicts the degradation of oligonucleotides 157 and 158 with SVPD as a function of incubation time and compared to degradation of an unmodified control oligonucleotide 159 as determined by CGE analysis.

FIG. 5 depicts the velocity of the hydrolysis of oligonucleotide 159 with BIPD as a function of the concentration of co-incubated oligonucleotides 158 and 158.

FIG. 6 depicts the percentage of a full-length L/D chimeric oligonucleotide that was present in various organs one hour after administration by IV bolus into BalbC mice.

FIG. 7 depicts the percentage of a full-length L/D chimeric oligonucleotide that was present in various organs twenty-four hours after administration by IV bolus into BalbC mice.

### DETAILED DESCRIPTION OF THE PREFERRED EMBODIMENTS

In the context of this invention, the terms “oligomer” and “oligomeric compound” refer to a plurality of naturally-occurring or non-naturally-occurring nucleosides joined together in a specific sequence. The terms “oligomer” and “oligomeric compound” include oligonucleotides, oligonucleotide analogs, oligonucleosides and chimeric oligomeric compounds where there are more than one type of internucleoside linkages dividing the oligomeric compound into regions. Oligomeric compounds are typically structurally distinguishable from, yet functionally interchangeable with, naturally-occurring or synthetic wild-type oligonucleotides. Thus, oligomeric compounds include all such structures that function effectively to mimic the structure and/or function of a desired RNA or DNA strand, for example, by hybridizing to a target.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for nucleic acid target and increased stability in the presence of nucleases.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure. However, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred oligomeric compounds useful in this invention include those having modified backbones or non-naturally occurring internucleoside linkages. As defined in this specification, modified backbones include those having a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

### Representative Phosphorus Containing Linkages

phosphorodithioate (—O—P(S)(S)—O—);

phosphorothioate (—O—P(S)(O)—O—);

phosphoramidate (—O—P(O)(NJ₂)—O—);

phosphonate (—O—P(J)(O)—O—);

phosphotriesters (—O—P(O J)(O)—O—);

phophosphoramidate (—O—P(O)(NJ)—S—);

thionoalkylphosphonate (—O—P(S)(J)—O—);

thionoalkylphosphotriester (—O—P(O)(OJ)—S—);

phosphoramidate (—N(J)—P(O)(O)—O—);

boranophosphate (—R⁵—P(O)(O)—J—);

where J denotes a substituent group which is commonly hydrogen or an alkyl group or a more complicated group that varies from one type of linkage to another.

Representative U.S. patents that teach the preparation of the above-noted phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified backbones that do not include a phosphorus atom therein are those that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Representative Non-Phosphorus Containing Linkages

thiodiester (—O—C(O)—S—);

thionocarbamate (—O—C(O)(NJ)—S—);

siloxane (—O—Si(J)₂—O—);

carbamate (—O—C(O)—NH— and —NH—C(O)—O—)

sulfamate (—O—S(O)(O)—N— and —N—S(O)(O)—N—;

morpholino sulfamide (—O—S(O)(N(morpholino)—);

sulfonamide (—O—SO₂—NH—);

sulfide (—CH₂—S—CH₂—);

sulfonate (—O—SO₂—CH₂—);

N,N′-dimethylhydrazine (—CH₂—N(CH₃)—N(CH₃)—);

thioformacetal (—S—CH₂—O—);

formacetal (—O—CH₂—O—);

thioketal (—S—C(J)₂—O—); and

ketal (—O—C(J)₂—O—);

amine (—NH—CH₂—CH₂—);

hydroxylamine (—CH₂—N(J)—O—);

hydroxylimine (—CH═N—O—); and

hydrazinyl (—CH₂—N(H)—N(H)—).

where J denotes a substituent group which is commonly hydrogen or an alkyl group or a more complicated group that varies from one type of linkage to another.

Representative U.S. patents that teach the preparation of the above-noted oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In certain preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Among the preferred compounds of this invention are oligonucleotides with phosphorothioate backbones and oligonucleotides with heteroatom backbones, and in particular —CH₂—NH—O—CH₂—, —CH₂—N(CH₃)—O—CH₂— [known as a methylene (methylimino), MMI backbone or more generally as methyleneimino], —CH2—O—N(CH₃)—CH₂—, —CH₂—N(CH₃)—N(CH₃)—CH₂— and —O—N(CH₃)—CH₂—CH₂— of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

“Bx,” as used herein, is intended to indicate a heterocyclic base moiety. Heterocyclic base moieties (often referred to in the art simply as a “bases” or a “nucleobases”) amenable to the present invention include naturally or non-naturally occurring nucleobases. One or more functionalities of the base can optionally bear a protecting group. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′, 2′:4,5]pyrrolo[2,3-d]pyrimidin-2-one). Modified nucleobases include those in which the purine or pyrimidine base is replaced with other heterocycles, for example 7-deaza-adenine, 7-deazaguanosine, 2-aminopyridine and 2-pyridone.

Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′—O—methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941, and 5,750,692, certain of which are commonly owned with the instant application, and each of which is herein incorporated by reference.

In one aspect of the present invention oligomeric compounds are prepared having one or more heterocyclic base moieties comprising a polycyclic heterocyclic base moiety. As used herein the term polycyclic heterocyclic base moiety is intended to include compounds comprising at least 3 or more fused rings. A number of tricyclic and some tetracyclic heterocyclic compounds have been prepared and substituted for naturally ocurring heterocyclic base moieties in oligomeric compounds. The resulting oligomeric compounds have been used in antisense applications to increase the binding properties of for example a modified strand to a target strand. The more studied modifications have been targeted to guanosines and are commonly referred to as cytidine analogs.

In one aspect of the present invention a polycyclic heterocyclic base moiety has the formula:

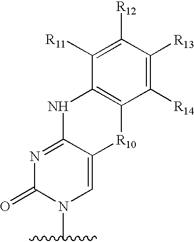

Representative cytosine analogs that make 3 hydrogen bonds with a guanosine in a second strand or elsewhere in the same strand include 1,3-diazaphenoxazine-2-one (R₁₀═O, R₁₁-R₁₄═H) [Kurchavov, et al., Nucleosides and Nucleotides, 1997, 16, 1837-1846], 1,3-diazaphenothiazine-2-one (R₁₀═S, R₁₁- R₁₄═H), [Lin, K. -Y.; Jones, R. J.; Matteucci, M. J. Am. Chem. Soc. 1995, 117, 3873-3874] and 6,7,8,9-tetrafluoro-1,3-diazaphenoxazine-2-one (R₁₀═O, R₁₁- R₁₄═F) [Wang, J.; Lin, K. -Y., Matteucci, M. Tetrahedron Lett. 1998, 39, 8385-8388]. Incorporated into oligo-nucleotides these base modifications were shown to hybridize with complementary guanine and the latter was also shown to hybridize with adenine and to enhance helical thermal stability by extended stacking interactions.

Further helix-stabilizing properties have been observed when a cytosine analogs having an aminoethoxy moiety attached to the rigid 1,3-diazaphenoxazine-2-one scaffold (R₁₀═O, R₁₁═—O—(CH₂)₂—NH₂, R₁₂₋₁₄═H, this analog has been given a particular name “G-clamp”) [Lin, K. -Y.; Matteucci, M. J. Am. Chem. Soc. 1998, 120, 8531-8532]. Binding studies demonstrated that a single incorporation could enhance the binding affinity of a model oligonucleotide to its complementary target DNA or RNA with a ΔTₘ of up to 18° C. relative to 5-methyl cytosine (dC5^{me}, which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further polycyclic heterocyclic base moieties and methods of using them that are amenable to the present invention are disclosed in U.S. Pat. Ser. Nos. 6,028,183, which issued on May 22, 2000, and 6,007,992, which issued on Dec. 28, 1999, the contents of both are commonly assigned with this application and are incorporated herein in their entirety. Such compounds include those having the formula:

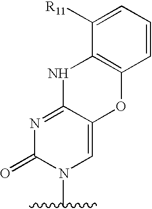

Wherein R₁₁ includes (CH₃)₂N—(CH₂)₂—O—; H₂N—(CH₂)₃—; Ph—CH₂—O—C(═O)—N(H)—(CH₂)₃—; H₂N—; Fluorenyl—CH₂—O—C(═O)—N(H)—(CH₂)₃—; Phthalimidyl—CH₂—O—C(═O)—N(H)—(CH₂)₃—; Ph—CH₂—O—C(═O)—N(H)—(CH₂)₂—O—; Ph—CH₂—O—C(═O)—N(H)—(CH₂)₃—O—; (CH₃)₂N—N(H)—(CH₂)₂—O—; Fluorenyl—CH₂—O—C(═O)—N(H)—(CH₂)₂—O—; Fluorenyl—CH₂—O—C(═O)—N(H)—(CH₂)₃—O—; H₂N—(CH₂)₂—O—CH₂—; N₃—(CH₂)₂—O—CH₂—; H₂N—(CH₂)₂—O—, and NH₂C(═NH)NH—.

Also disclosed are polycyclic heterocyclic compounds of the formula:

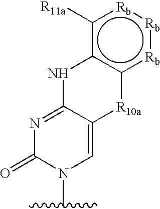

Wherein

R₁₀ₐ is O, S or N—CH₃;

R₁₁ₐ is A(Z)ₓ₁,wherein A is a spacer and Z independently is a label bonding group bonding group optionally bonded to a detectable label, but R₁₁ₐ is not amine, protected amine, nitro or cyano;

X1 is 1, 2 or3; and

R_{b} is independently —CH═, —N═, —C(C₁₋₈alkyl)═ or —C(halogen)═, but no adjacent R_{b} are both —N═, or two adjacent R_{b} are taken together to form a ring having the structure:

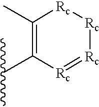

where R_{c} is independently —CH═, —N═, —C(C₁₋₈alkyl)═ or —C(halogen)═, but no adjacent R_{b} are both —N═.

The enhanced binding affinity of the phenoxazine derivatives together with their uncompromised sequence specificity makes these polycyclic heterocyclic base moieties valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity [Lin, K. -Y.; Matteucci, M. J. Am. Chem. Soc. 1998, 120, 8531-8532]. The activity enhancement was even more pronounced when the heterocyclic heterocyclic base moiety was the “G-clamp” where a single substitution was shown to significantly improve the in vitro potency of 20 mer 2′-deoxyphosphorothioate oligonucleotides [Flanagan, W. M.; Wolf, J. J.; Olson, P.; Grant, D.; Lin, K. -Y.; Wagner, R. W.; Matteucci, M. Proc. Natl. Acad. Sci. U.S.A., 1999, 96, 3513-3518]. Nevertheless, to optimize oligonucleotide design and to better understand the impact of these polycyclic heterocyclic base modifications on biological activity, it is important to evaluate their effect on nuclease stability of the oligomers.

Further polycyclic heterocyclic base moieties comprising tricyclic and tetracyclic heteroaryl compounds amenable to the present invention include those having the formulas:

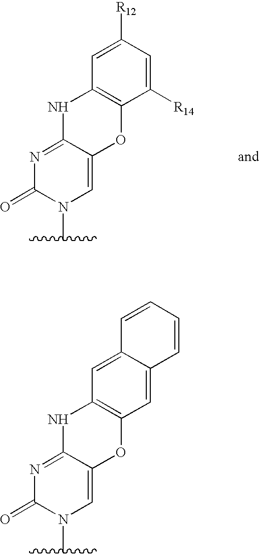

wherein R₁₄ is NO₂ or both R₁₄ and R₁₂ are independently —CH₃. The synthesis of these compounds is dicslosed in U.S. Pat. Ser. Nos. 5,434,257, which issued on Jul. 18, 1995, 5,502,177, which issued on Mar. 26, 1996, and 5,646,269, which issued on Jul. 8, 1997, the contents of which are commonly assigned with this application and are incorporated herein in their entirety.

Further polycyclic heterocyclic base moieties amenable to the present invention also disclosed in the “257, 177 and 269” Patents include those having the formula:

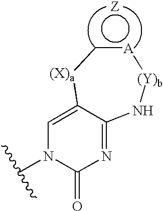

a and b are independently 0 or 1 with the total of a and b being 0 or 1;

A is N, C or CH;

X is S, O, C═O, NH or NCH₂, R⁶;

Y is C═O;

Z is taken together with A to form an aryl or heteroaryl ring structure comprising 5 or 6 ring atoms wherein the heteroaryl ring comprises a single O ring heteroatom, a single N ring heteroatom, a single S ring heteroatom, a single O and a single N ring heteroatom separated by a carbon atom, a single S and a single N ring heteroatom separated by a C atom, 2 N ring heteroatoms separated by a carbon atom, or 3 N ring heteroatoms at least 2 of which are separated by a carbon atom, and wherein the aryl or heteroaryl ring carbon atoms are unsubstituted with other than H or at least 1 nonbridging ring carbon atom is fubstituted with R²⁰ or ═O;

or Z is taken together with A to form an aryl ring structure comprising 6 ring atoms wherein the aryl ring carbon atoms are unsubstituted with other than H or at least 1 nonbridging ring carbon atom is substituted with R⁶ or ═O;

R⁶ is independently H, C₁₋₆alkyl, C₂₋₆alkenyl, C₂₋₆alkynyl, NO₂, N(R³)₂, CN or halo, or an R⁶ is taken together with an adjacent Z group R⁶ to complete a phenyl ring;

R²⁰ is, independently, H, C₁₋₆alkyl, C₂₋₆alkyl, C₂₋₆alkenyl, C₂₋₆alkynyl, NO₂, N(R²¹)₂, CN, or halo, or an R²⁰ is taken together with an adjacent R²⁰ to complete a ring containing 5 or 6 ring atoms, and tautomers, solvates and salts thereof;

R²¹ is, independently, H or a protecting group;

R³ is a protecting group or H; and tautomers, solvates and salts thereof.

More specific examples included in the “257, 177 and 269” Patents are compounds of the formula:

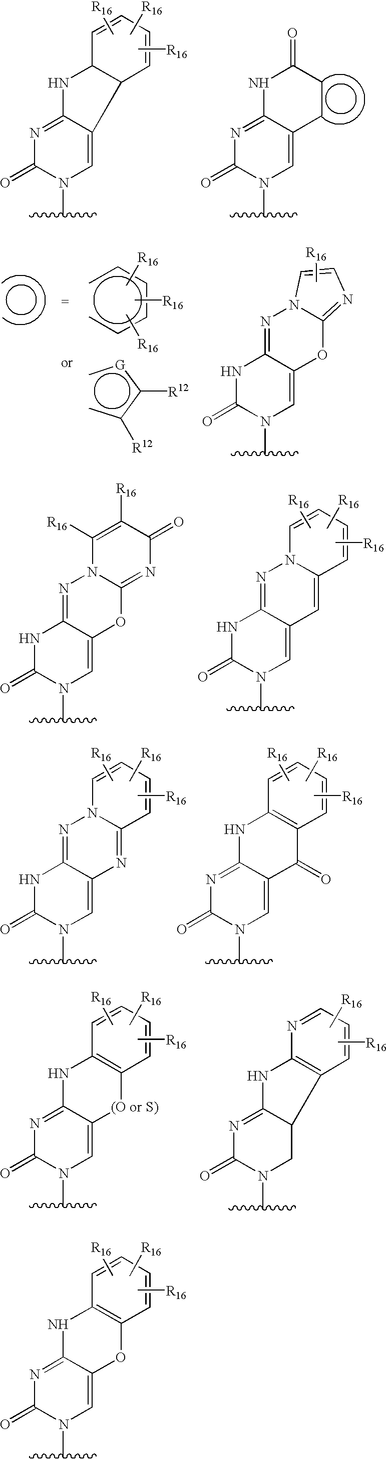

wherein each R₁₆, is, independently, selected from hydrogen and various substituent groups.

The present invention provides oligomeric compounds comprising a plurality of linked nucleosides wherein the preferred internucleoside linkage is a 3′, 5′-linkage. Alternatively, 2′, 5′-linkages can be used (as described in U.S. application Ser. No. 09/115,043, filed Jul. 14, 1998). A 2′, 5′-linkage is one that covalently connects the 2′-position of the sugar portion of one nucleotide subunit with the 5′-position of the sugar portion of an adjacent nucleotide subunit.

The compounds described herein may have asymmetric centers. Unless otherwise indicated, all chiral, diastereomeric, and racemic forms are included in the present invention. Geometric isomers may also be present in the compounds described herein, and all such stable isomers are contemplated by the present invention. It will be appreciated that compounds in accordance with the present invention that contain asymmetrically substituted carbon atoms may be isolated in optically active or racemic forms or by synthesis.

The present invention includes all isotopes of atoms occurring in the intermediates or final compounds. Isotopes include those atoms having the same atomic number but different mass numbers. By way of example, and without limitation, isotopes of hydrogen include tritium and deuterium.

As used herein, the term “sugar substituent group” refers to optionally protected groups that are attached to selected sugar moieties at the 2′, 3′, or 5′-position. Sugar substituent groups have also been attached to heterocyclic base moieties for example by attachment at amino functionalities.

A representative list of sugar substituent groups amenable to the present invention include hydroxyl, C₁-C₂₀ alkyl, C₂-C₂₀ alkenyl, C₂-C₂₀ alkynyl, C₅-C₂₀ aryl, O-alkyl, O-alkenyl, O-alkynyl, O-alkylamino, O-alkylalkoxy, O-alkylaminoalkyl, O-alkyl imidazole, S-alkyl, S-alkenyl, S-alkynyl, NH-alkyl, NH-alkenyl, NH-alkynyl, N-dialkyl, O-aryl, S-aryl, NH-aryl, O-aralkyl, S-aralkyl, NH-aralkyl, N-phthalimido, halogen (particularly fluoro), amino, thiol, keto, carboxyl, nitro, nitroso, nitrile, trifluoromethyl, trifluoromethoxy, imidazole, azido, hydrazino, hydroxylamino, isocyanato, sulfoxide, sulfone, sulfide, disulfide, silyl, aryl, heterocycle, carbocycle, intercalators, reporter groups, conjugates, polyamine, polyamide, polyalkylene glycol, and polyethers of the formula (O-alkyl)ₘ, where m is 1 to about 10. Preferred among these polyethers are linear and cyclic polyethylene glycols (PEGs), and (PEG)-containing groups, such as crown ethers and those which are disclosed by Ouchi et al. (Drug Design and Discovery 1992, 9, 93), Ravasio et al. (J. Org. Chem. 1991, 56, 4329) and Delgardo et. al. (Critical Reviews in Therapeutic Drug Carrier Systems 1992, 9, 249), each of which is herein incorporated by reference in its entirety. Further sugar modifications are disclosed in Cook, P. D., Anti-Cancer Drug Design, 1991, 6, 585-607. Fluoro, O-alkyl, O-alkylamino, O-alkyl imidazole, O-alkylaminoalkyl, and alkyl amino substitution is described in U.S. patent application Ser. No. 08/398,901, filed Mar. 6, 1995, entitled Oligomeric Compounds having Pyrimidine Nucleotide(s) with 2′ and 5′ Substitutions, hereby incorporated by reference in its entirety.

Additional sugar substituent groups amenable to the present invention include —SR₁ and —N(R₁)₂ groups, wherein each R₁ is, independently, hydrogen, a protecting group or substituted or unsubstituted alkyl, alkenyl, or alkynyl. 2′—S—R, nucleosides are disclosed in U.S. Pat. No. 5,670,633, issued Sep. 23, 1997, hereby incorporated by reference in its entirety. The incorporation of 2′-S R₁ monomer synthons are disclosed by Hamm et al., J. Org. Chem., 1997, 62, 3415-3420. 2′-N(R₁)₂ nucleosides are disclosed by Goettingen, M., J. Org. Chem., 1996, 61, 6273-6281; and Polushin et al., Tetrahedron Lett., 1996, 37, 3227-3230.

Further representative sugar substituent groups can include groups having the structure of one of formula I or II:

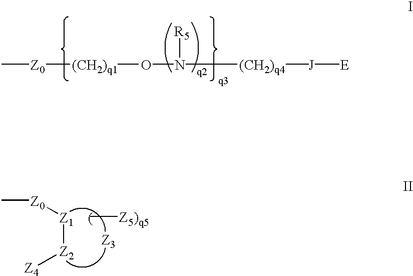

wherein:

Z₀ is O, S or NH;

J is a single bond, O or C(═O);

E is C₁-C₁₀alkyl, N(R₅)(R₆), N(R₅)(R₇), N═C(R₅ₐ)(R₆ₐ), N═C(R₅ₐ)(R₇ₐ) or has formula III;

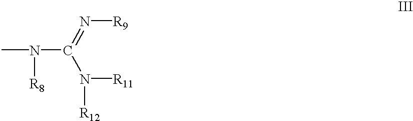

each R₈, R₉, R₁₁ and R₁₂ is, independently, hydrogen, C(O)R₁₃, substituted or unsubstituted C₁-C₁₀ alkyl, substituted or unsubstituted C₂-C₁₀alkenyl, substituted or unsubstituted C₂-C₁₀alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, R₁ and R₁₂, together form a phthalimido moiety with the nitrogen atom to which they are attached;

each R₁₃ is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

R₅ is hydrogen, a nitrogen protecting group or —T—L,

R₅ₐ is hydrogen, a nitrogen protecting group or —T—L,

T is a bond or a linking moiety;

L is a chemical functional group, a conjugate group or a solid support material;

each R₆ and R₇ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein said substitution is hydroxyl, amino, alkoxy, carboxy, benzyl, phenyl, nitro, thiol, thioalkoxy, halogen, alkyl, aryl, alkenyl, alkynyl; NH₃⁺, N(R₁₄)(R₁₅), guanidino or acyl where said acyl is an acid amide or an ester;

or R₆ and R₇, together, are a nitrogen protecting group, are joined in a ring structure that optionally includes an additional heteroatom selected from N and O or are a chemical functional group;

each R₁₄ and R₁₅ is, independently, H, C₁-C₁₀ alkyl, a nitrogen protecting group, or R₁₄ and R₁₅, together, are a nitrogen protecting group;

or R₁₄ and R₁₅ are joined in a ring structure that optionally includes an additional heteroatom selected from N and O;

Z₄ is OX, SX, or N(X)₂;

each X is, independently, H, C₁-C₈ alkyl, C₁-C₈ haloalkyl, C(═NH)N(H)R₁₆, C(═O)N(H)R₁₆ or OC(═O)N(H)R₁₆;

R₁₆ is H or C₁-C₈ alkyl;

Z₁, Z₂ and Z₃ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Z₅ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(R₅)(R₆) OR₅, halo, SR₅ or CN;

each q₁ is, independently, an integer from 1 to 10;

each q₂ is, independently, 0 or 1;

q₃ is 0 or an integer from 1 to 10;

q₄ is an integer from 1 to 10;

q₅ is from 0, 1 or 2; and

provided that when q₃ is 0, q₄ is greater than 1.

Representative sugar substituent groups of Formula I are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic sugar substituent groups of Formula II are disclosed in U.S. patent application Ser. No. 09/123,108, filed Jul. 27, 1998, entitled “RNA Targeted 2′-Modified Oligonucleotides that are Conformationally Preorganized,” hereby incorporated by reference in its entirety.

Particularly preferred sugar substituent groups include O[(CH₂)ₚ₁O]ₚ₂CH₃, O(CH₂)ₚ₁OCH₃, O(CH₂)ₚ₁NH₂, O(CH₂)ₚ₁CH₃, O(CH₂)ₚ₁ONH₂, and O(CH₂) ₚ₁ON[(CH₂)ₚ₁CH₃)]₂, where p1 and p2 are from 1 to about 10.

Some preferred oligomeric compounds of the invention contain at least one nucleoside having one of the following sugar substituent groups: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligomeric compound, or a group for improving the pharmacodynamic properties of an oligomeric compound, and other sugar substituent groups having similar properties. A preferred modification includes 2′-methoxyethoxy [2′—O—CH₂CH₂OCH₃, also known as 2′—O—(2-methoxyethyl) or 2′-MOE] (Martin et al., Helv. Chim. Acta, 1995, 78, 486), i.e., an alkoxyalkoxy group. A further preferred modification is 2′-dimethylaminooxyethoxy, i.e., a O(CH₂)₂ON(CH₃)₂ group, also known as 2′-DMAOE. Representative aminooxy sugar substituent groups are described in co-owned U.S. patent application Ser. No. 09/344,260, filed Jun. 25, 1999, entitled “Aminooxy-Functionalized Oligomers”; and U.S. patent application Ser. No. 09/370,541, filed Aug. 9, 1999, entitled “Aminooxy-Functionalized Oligomers and Methods for Making Same;” hereby incorporated by reference in their entirety.

Other preferred modifications include 2′-methoxy (2′—O—CH₃), 2′-aminopropoxy (2′—OCH₂CH₂CH₂NH₂) and 2′-fluoro (2′-F). Similar modifications may also be made at other positions on nucleosides and oligomers, particularly the 3′ position of the sugar on the 3′ terminal nucleoside or at a 3′-position of a nucleoside that has a linkage from the 2′-position such as a 2′-5′ linked oligomer and at the 5′ position of a 5′ terminal nucleoside. Oligomers may also have sugar mimetics such as cyclobutyl moieties in place of the pentofuranosyl sugar. Representative U.S. patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920, certain of which are commonly owned, and each of which is herein incorporated by reference, and commonly owned U.S. patent application Ser. No. 08/468,037, filed on Jun. 5, 1995, also herein incorporated by reference.

Representative guanidino sugar substituent groups that are shown in formula III are disclosed in co-owned U.S. patent application Ser. No. 09/612,531, entitled “Guinidinium Functionalized Oligomers and Methods”, filed Jul. 7, 2000, hereby incorporated by reference in its entirety.

Representative acetamido sugar substituent groups are disclosed in U.S. patent application Ser. No. 09/378,568, entitled “2′—O—Acetamido Modified Monomers and Oligomers”, filed Aug. 19, 1999, hereby incorporated by reference in its entirety.

Representative dimethylaminoethyloxyethyl sugar substituent groups are disclosed in International Patent Application PCT/US99/17895, entitled “2′—O—Dimethylaminoethyloxyethyl-Modified Oligonucleotides”, filed Aug. 6, 1999, hereby incorporated by reference in its entirety.

A further prefered modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

It is not necessary for all positions in a given compound to be uniformly modified, and in fact more than one of the aforementioned modifications may be incorporated in a single compound or even at a single nucleoside within an oligonucleotide.

The present invention also includes oligomeric compounds that are chimeric compounds. “Chimeric” oligomeric compounds or “chimeras,” in the context of this invention, are oligomeric compounds, particularly oligonucleotides, that contain two or more chemically distinct regions, each made up of at least one monomer unit, i.e., a nucleotide in the case of an oligonucleotide compound. Chimeric oligonucleotides typically contain at least one region wherein the oligonucleotide is modified so as to confer increased resistance to nuclease degradation, increased cellular uptake, and/or increased binding affinity for the target nucleic acid upon the oligonucleotide. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

In certain embodiments, the oligomeric compounds of the invention can be chimeric oligonucleotides, including “gapmers,” “inverted gapmers,” or “hemimers.” In a “hemimer,” a single terminal (either 5′ or 3′) region of the oligonucleotide contains modified nucleosides. When both termini of the oligonucleotide contain modified nucleosides, the oligonucleotide is called a “gapmer” and the modified 5′- and 3′-terminal regions are referred to as “wings”. In a gapmer, the 5′ and 3′ wings can contain nucleosides modified in the same or different manner. In an “inverted gapmer” a central region of the oligonucleotide contains modified nucleosides. The present invention provides compounds and methods that are useful for enhancing the nuclease resistance of oligomeric compounds. More specifically, the present invention is directed to oligomeric compounds that exhibit enhanced nuclease resistance, and to methods for improving the nuclease stability of oligomeric compounds. As noted above, resistance to enzymatic degradation is an important feature of antisense oligonucleotide therapeutics, and the efficacy of antisense oligonucleotide drugs has been hampered by the activity of nucleases present in biological systems. Surprisingly, it has been discovered that certain modifications of oligomeric compounds enhance their nuclease stability. Novel methods for increasing the nuclease stability of oligomeric compounds involving the incorporation of modified nucleosides have also been discovered.

The present invention is directed to nuclease-resistant oligomeric compounds that may be useful as pharmaceuticals. Antisense oligonucleotides can be designed to bind in predictable ways to certain nucleic acid target sequences, which can cause selective inhibition of the expression of genes whose products lead to disease. Antisense oligonucleotides can bind to specific complementary regions on mRNA, thereby inhibiting protein biosynthesis through the disruption of processes such as splicing, polyadenylation, correct RNA folding, translocation and initiation of translation of mRNA, or ribosome movement along the mRNA. The oligomeric compounds of the invention typically exhibit enhanced nuclease resistance and can be used as effective antisense oligonucleotides in therapeutic applications for the treatment of specific diseases. The methods of the invention can also be used to increase the efficacy of antisense oligonucleotides as therapeutics through enhancement of the nuclease resistance of oligomeric compounds.

Preferred embodiments of the invention include nuclease resistant oligomeric compounds that comprise at least one modified 5′ or 3′ terminal nucleoside or nucleotide and at least one internucleoside linking group other than phosphodiester, and optionally comprise modified 2′ substituent groups in the gapmer, hemimer, and inverted gapmer configuration and one or more modified nucleobases.

The tricyclic cytosine analogs phenoxazine and 9-(aminoethoxy)phenoxazine (G-clamp) have been shown to significantly enhance the nuclease resistance of oligonucleotides. Phenoxazine and G-clamp were incorporated into model oligomers with a natural phosphodiester backbone and enzymatic degradation was monitored after treatment with snake venom phosphodiesterase. A single incorporation of either phenoxazine or G-clamp at the 3′ terminus completely protected the oligonucleotides against 3′ exonuclease attack. The nuclease resistance of oligonucleotides containing phenoxazine and G-clamp is not believed to be caused by low binding affinity for the enzyme's active site, as the modified oligonucleotides are capable of slowing down the degradation of a natural DNA fragment by bovine intestinal mucosal phosphodiesterase in a dose-dependent manner. No significant difference was observed between phenoxazine and G-clamp in terms of their effects on nuclease resistance and their capacity to inhibit nuclease activity.

A guanidinyl moiety can be added to an oligonucleotide by postsynthetic guanidinylation of a primary amino group tethered to either the 2′-position or to the phenoxazine ring system of a tricyclic cytosine analog (G-clamp). The former amino group can be selectively deprotected and guanidinylated on the solid support, while the aminoethoxy tether of G-clamp can be guanidinylated in aqueous solution after deprotection and cleavage of the oligonucleotide from the support. Both methods have been successfully used to synthesize and characterize various guanidinyl-modified oligonucleotides. The conversion of a primary amine to a guanidinium moiety, which has a significantly higher pKₐ than a primary amine, allows a positive charge to be introduced to the oligonucleotide, which is maintained over a wide pH range. The introduction of cationic residues at the 2′-position greatly enhances the nuclease resistance of oligonucleotides (Prakash, T. P.; Kawasaki, A. M.; Vasquez, G.; Fraser, A. S.; Casper, M. D.; Cook, P. D.; Manoharan, M. Nucleosides Nucleotides 1999, 18, 1381-1382). X-ray crystallography studies of a decamer duplex containing guanidinyl G-clamp nucleotides revealed an additional Hoogsteen bond between the imino or amino nitrogens of the tethered guanidinium and N7 of a complementary guanine base, which was the first observation of a single base pair within a nucleic acid duplex containing a total number of five hydrogen bonds.

The current method of choice for the preparation of oligomeric compounds uses support media. Support media is used to attach a first nucleoside or larger nucleosidic synthon which is then iteratively elongated to give a final oligomeric compound. Support media can be selected to be insoluble or have variable solubility in different solvents to allow the growing oligomer to be kept out of or in solution as desired. Traditional solid supports are insoluble and are routinely placed in a reaction vessel while reagents and solvents react and or wash the growing chain until cleavage frees the final oligomer. More recent approaches have introduced soluble supports including soluble polymer supports to allow precipitating and dissolving the bound oligomer at desired points in the synthesis (Gravert et al., Chem. Rev., 1997, 97, 489-510).

Representative support media that are amenable to the methods of the present invention include without limitation: controlled pore glass (CPG); oxalyl-controlled pore glass (see, e.g., Alul, et al., Nucleic Acids Research 1991, 19, 1527); TENTAGEL Support, (see, e.g., Wright, et al., Tetrahedron Letters 1993, 34, 3373); or POROS, a copolymer of polystyrene/divinylbenzene available from Perceptive Biosystems. The use of a soluble support media, poly(ethylene glycol), with molecular weights between 5 and 20 kDa, for large-scale synthesis of phosphorothioate oligonucleotides is described in, Bonora et al., Organic Process Research & Development, 2000, 4, 225-231. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Activated phosphorus compositions (e.g. compounds having activated phosphorus-containing substituent groups) may be used in coupling reactions for the synthesis of oligomeric compounds. As used herein, the term “activated phosphorus composition” includes monomers and oligomers that have an activated phosphorus-containing substituent group that is reactive with a hydroxyl group of another monomeric or oligomeric compound to form a phosphorus-containing internucleotide linkage. Such activated phosphorus groups contain activated phosphorus atoms in p^{III} valence state. Such activated phosphorus atoms are known in the art and include, but are not limited to, phosphoramidite, H-phosphonate, phosphate triesters and chiral auxiliaries. A preferred synthetic solid phase synthesis utilizes phosphoramidites as activated phosphates. The phosphoramidites utilize P^{III} chemistry. The intermediate phosphite compounds are subsequently oxidized to the P^{V} state using known methods to yield, in a preferred embodiment, phosphodiester or phosphorothioate internucleotide linkages. Additional activated phosphates and phosphites are disclosed in Tetrahedron Report Number 309 (Beaucage and Iyer, Tetrahedron, 1992, 48, 2223-2311).

A representative list of activated phosphorus containing monomers or oligomers include those having the formula:

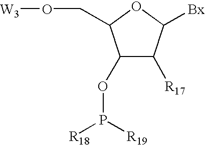

wherein

each Bx is, independently, a heterocyclic base moiety or a blocked heterocyclic base moiety; and

each R₁₇ is, independently, H, a blocked hydroxyl group, a sugar substituent group, or a blocked substituent group;

W₃ is an hydroxyl protecting group, a nucleoside, a nucleotide, an oligonucleoside or an oligonucleotide;

R₁₈ is N(L₁)L₂;

each L₁ and L₂ is, independently, C₁₋₆alkyl;

or L₁ and L₂ are joined together to form a 4- to 7-membered heterocyclic ring system including the nitrogen atom to which L₁ and L₂ are attached, wherein said ring system optionally includes at least one additional heteroatom selected from O, N and S; and

R₁₉ is X₁;

X₁ is Pg—O—, Pg—S—, C₁-C₁₀ straight or branched chain alkyl, CH₃(CH₂)ₚ₅—O— or

R₂₀R₂₁N—;

p⁵ is from 0 to 10;

Pg is a protecting group;

each R₂₀ and R₂₁ is, independently, hydrogen, C₁-C₁₀alkyl, cycloalkyl or aryl;

or optionally, R₂₀ and R₂₁, together with the nitrogen atom to which they are attached form a cyclic moiety that may include an additional heteroatom selected from O, S and N; or

R₁₈ and R₁₉ together with the phosphorus atom to which R₁₈ and R₁₉ are attached form a chiral auxiliary.

Groups that are attached to the phosphorus atom of internucleotide linkages before and after oxidation (R₁₈ and R₁₉) can include nitrogen containing cyclic moieties such as morpholine. Such oxidized internucleotide linkages include a phosphoromorpholidothioate linkage (Wilk et al., Nucleosides and nucleotides, 1991, 10, 319-322). Further cyclic moieties amenable to the present invention include mono-, bi- or tricyclic ring moieties which may be substituted with groups such as oxo, acyl, alkoxy, alkoxycarbonyl, alkyl, alkenyl, alkynyl, amino, amido, azido, aryl, heteroaryl, carboxylic acid, cyano, guanidino, halo, haloalkyl, haloalkoxy, hydrazino, ODMT, alkysulfonyl, nitro, sulfide, sulfone, sulfonamide, thiol and thiolalkyoxy. A preferred bicyclic ring structure that includes nitrogen is phthalimido.

In the context of this specification, alkyl (generally C1-C20), alkenyl (generally C2-C20), and alkynyl (generally C2-C20) groups include but are not limited to substituted and unsubstituted straight chain, branch chain, and alicyclic hydrocarbons, including methyl, ethyl, propyl, butyl, pentyl, hexyl, heptyl, octyl, nonyl, decyl, undecyl, dodecyl, tridencyl, tetradecyl, pentadecyl, hexadecyl, heptadecyl, octadecyl, nonadecyl, eicosyl and other higher carbon alkyl groups. Further examples include 2-methylpropyl, 2-methyl-4-ethylbutyl, 2,4-diethylbutyl, 3-propylbutyl, 2,8-dibutydecyl, 6,6-dimethylocyl, 6-propyl-6-butyloctyl, 2-methyl-butyl, 2-methylpentyl, 3-methylpentyl, 2-ethylhexyl and other branched chain groups, allyl, crotyl, propargyl, 2-pentenyl and other unsaturated groups containing a pi bond, cyclohexane, cyclopentane, adamantane as well as other alicyclic groups, 3-penten-2-one, 3-methyl-2-butanol, 2-cyanooctyl, 3-methoxy-4-heptanal, 3-nitrobutyl, 4-isopro-poxydodecyl, 4-azido-2-nitrodecyl, 5-mercaptononyl, 4-amino-1-pentenyl as well as other substituted groups. Representative alkyl substituents are disclosed in U.S. Pat. No. 5,212,295, at column 12, line 41-50, hereby incorporated by reference in its entirety.

A number of chemical functional groups can be introduced into compounds of the invention in a blocked form and subsequently deblocked to form a final, desired compound. Such as groups directly or indirectly attached at the heterocyclic bases, the internucleosides linkages and the sugar substituent groups at one or more or the 2′, 3′ and 5′-positions. Protecting groups can be selected to block functional groups located in a growing oligomeric compound during iterative oligonucleotide synthesis while other positions can be selectively deblocked as needed. In general, a blocking group tenders a chemical functionality of a larger molecule inert to specific reaction conditions and can later be removed from such functionality without substantially damaging the remainder of the molecule (Greene and Wuts, Protective Groups in Organic Synthesis, 3rd ed, John Wiley & Sons, New York, 1999). For example, the nitrogen atom of amino groups can be blocked as phthalimido groups, as 9-fluorenylmethoxycarbonyl (FMOC) groups, and with triphenylmethylsulfenyl, t-BOC or benzyl groups. Carboxyl groups can be blocked as acetyl groups. Representative hydroxyl protecting groups are described by Beaucage et al., Tetrahedron 1992, 48, 2223. Preferred hydroxyl protecting groups are acid-labile, such as the trityl, monomethoxytrityl, dimethoxytrityl, trimethoxytrityl, 9-phenylxanthine-9-yl (Pixyl) and 9-(p-methoxyphenyl)xanthine-9-yl (MOX).

Chemical functional groups can also be “blocked” by including them in a precursor form. Thus, an axido group can be used considered as a “blocked” form of an amine since the axido group is easily converted to the amine. Further representative protecting groups utilized in oligonucleotide synthesis are discussed in Agrawal, et al., Protocols for Oligonucleotide Conjugates, Eds, Humana Press; New Jersey, 1994; Vol. 26 pp. 1-72.

Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilyethyl, p-chlorophenyl, 2,4-dinitrophenyl, benzyl, 2,6-dichlorobenzyl, diphenylmethyl, p,p═dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, privaloate, benzoate, p-phenylbenzoate, 9-fluoroenylmethyl carbonate, mesylate and tosylate.

Examples of thiol (sulfur) protecting groups include, but are not limited to, benzyl, substituted benzyls, diphenylmethyl, phenyl, t-butyl, methoxymethyl, thiazolidines, acetyl and benzoyl. Further thiol protecting groups are illustrated in Greene and Wuts, ibid.

Additional amino-protecting groups include but are not limited to, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide-protecting groups, such as formyl, acetyl, trihaloacetyl, benzoyl, and nitrophenylacetyl; sulfonamide-protecting groups, such as 2-nitrobenzenesulfonyl; and imine- and cyclic imide-protecting groups, such as phthalimido and dithiasuccinoyl. Equivalents of these amino-protecting groups are also encompassed by the compounds and methods of the present invention.

Some preferred amino-protecting groups are stable to acid treatment and can be selectively removed with base treatment which make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p.1), and various substituted sulfonylethyl carbamates exemplified by the Nsc group (Samukov et al., Tetrahedron Lett, 1994, 35:7821; Verhart and Tesser, Rec. Trav. Chim. Pays-Bas, 1987, 107:621).

In some especially preferred embodiments, the nucleoside components of the oligomeric compounds are connected to each other by optionally protected phosphorothioate internucleoside linkages. Representative protecting groups for phosporus containing internucleoside linkages. Representative protecting groups for phosphorus containing internucleosides linkages such as phosphite, phosphodiester and phosphorothioate linages includes β-cyanoethyl, diphenylsilylethyl, δ- cyanobutenyl, cyano p-xylyl (CPX), N-methyl-N-trifluoroacetyl ethyl (META), acetoxy phenoxy ethyl (APE) and butene-4-yl groups. See for example U.S. Pat. Nos. 4,725,677 and Re. 34,069 (β-cyanoethyl); Beaucage, S. L. and Iyer, R. P., Tetrahedron, 49 No. 10, pp. 1925-1963 (1993); Beaucage, S. L. and Iyer, R. P., Tetrahedron, 49 No. 46, pp. 10441-10488 (1993); Beaucage, S. L. and Iyer, R. P., Tetrahedron, 48 No. 12, pp.

The present invention also includes pharmaceutical compositions and formulations that include the oligomeric compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated codoms, gloves and the like may also be useful. Preferred topical formulations include those in which the oligomeric compounds of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine (DOTMA). Oligomeric compounds of the invention may be encapsulated within lipsomes or may form complexes thereto, in particular to cationic liposomes. Alternatively, oligomeric compounds may be complexed to lipids, in particular to cationic lipids. Preferred fatty acids and esters include but are not limited arachidonic acid, oleic acid, eicosanoic acid, lauric acid, linolenic acid, dicaprate, tricaprate, monoolein, dilaurin, glyceryl 1-monocaprate, 1-dodecylazacycloheptan-2-one, and acylcarnitine, an acylcholine, or a C₁₋₁₀alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitables. Thickeners, flavoring agents, diluents, emulsifiers, dispersing aids or binders may be desirable. Preferred oral formulations are those in which oligomeric compounds of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligomeric compounds of the invention may be delivered orally in granular form including sprayed dried particles, or complexed to form micro or nonoparticles. Oligonucleotides complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-latic acid), poly(DL-lactic-co-glycolic acid (PLGA), alginate, and polyethyleneglycols (PEG). Oral formulations for oligonucleotides and their preparation are described in detail in U.S. applications Ser. Nos. 08/886,829 (filed Jul. 1, 1997), 09/108,673 (filed Jul. 1, 1998), 09/256,515 (filed Feb. 23, 1999), 09/082,624 (filed May 21, 1998) and 09/315,298 (filed May 20, 1999) each of which is incorporated herein by reference in their entirety.

Compositions and formulations for parenteral, intrathecal or intraventricular administrations may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation in such compositions and formulations is generally known to those skilled in the pharmaceutical and formulations arts and may be applied to the formulation of the compositions of the present invention.

The compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories synthetic surfactants, naturally occurring emulsifiers, absorption bases, and finely dispersed solids (Idson, in Pharmaceutically Dosage Forms, Lieberman, Rieger and Banker (Eds.), 1988, Marcel Dekker, Inc., New York, N.Y., volume 1, p. 199).

Synthetic surfactants, also known as surface agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a variable tool in categorizing and selecting surfactants in the preparation of formulations. Surfactants may be classified into different classes based on the nature of the hydrophilic group; nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199). Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin. The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligomeric compounds and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described in thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York pages 185-215). Microemulsions commonly are prepared via a combination of three to five components that include oil, water, surfactant, cosurfactant and electrolyte. Whether the microemulsions is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsions systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13,205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligomeric compounds and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Lipsomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administrative drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun. 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phosphilids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxytethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P.Pharma. Sci., 1994, 4, 6, 466).

Lipsomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765). Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, eg., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pt. No. 5,225,212 (both to Martin et al.) and in WO 94/20073 (Zalipsky et al.) Liposomes comprising PEG-modified ceramide lipids are described in WO 96/10391 (Choi et al.). U.S. Pat. Nos. 5,540,935 (Miyazaki et al.) and 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also includes in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium slats and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivates, substituted alkylamides, N-alkylbetaines and phosphatides. The use of surfactants in drug products, formulations and in emulsions has been reviewed (Rieger, in Pharmaceutical Dosage Forms, Marcel Dekker, Inc., New York, N.Y., 1988, p. 285).

In one embodiment, the present invention employs various penetration enhancers to effect the efficient delivery of nucleic acids, particularly oligomeric compounds, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs. Penetration enhancers may be classified as belonging to one of five broad categories. i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al. J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monoleoyl-rac-glycerol), dilaurin, caprylic acid, arachidonic acid, glycerol 1-monocaprate, 1-dodecylazacycloheptan-2-one, acylcarnitines, acylcholines, C₁₋₁₀alkyl esters therof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glyodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-aklanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides as the cellular level may also be added to the pharmaceutical and other compositions of the present invention. For example, cationic lipids, such as lipofectin (Junichi et al., U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drugs Dev., 1996, 6, 177-183).

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutically compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms or the compositions of the present invention, such as dyes, flavoring agents, preservatives, antioxidante, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative U.S. patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The oligomeric compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention; i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci. 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligomeric compounds, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The materials, methods, and examples presented herein are intended to be illustrative, and are not intended to limit the scope of the invention. All publications, patent applications, patents, and other references mentioned herein are incorporated by reference in their entirety. Unless otherwise defined, all technical and scientific terms are intended to have their art-recognized meanings.

### EXAMPLE 1

### 5′-O-DMT-L-thymidine (2)

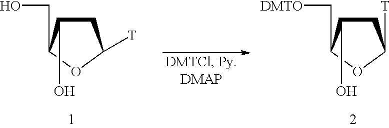

Compound 1 (800 mg, 3.3 mmol, (prepared according to Smejkal, J. et. al. Collect. Czech. Chem. Commun. 1964, 29, 2809-2813 and Jung, M. E. et al. Tetrahedron Lett. 1998, 39, 4615-4618) was dried over P₂O₅ under high vacuum overnight at 40° C. It was then co-evaporated with anhydrous pyridine (10 mL). The residue obtained was dissolved in pyridine (9 mL) under an argon atmosphere. 4-Dimethylaminopyridine (40 mg, 0.33 mmol), and DMT chloride (DMT-Cl, 1.33 g, 3.93 mmol) were added to the mixture and the reaction mixture was stirred at room temperature until all of the starting material disappeared (12 h). Methanol (0.5 mL) was added and solvent was removed in vacuo. The residue was chromatographed and eluted with ethyl acetate: exane, 6:4 to give 2 (1.42 g, 79%). R_{f}=0.17 (with ethyl acetate:hexane, 6:4). MS(ES⊕) m/z 543 (M−H).

### EXAMPLE 2

### 5′-O-DMT-L-thymidine-3′-O-[(2-cyanoethyl)-N,N-diisopropylphosphoramidite] (3)

Compound 2 (1.00 g, 1.84 mmol) was co-evaporated with toluene (20 mL). To the residue N,N-diisopropylamine tetrazolide (0.16 g, 0.92 mmol) was added and dried over P₂O₅ under high vacuum overnight at 40° C. The dried reaction mixture was dissolved in anhydrous acetonitrile: CH₂Cl₂ (9:2 mL) and 2-cyanoethyl-N,N,N′,N′-tetraisopropylphosphoramidite 1.2 mL, 3.68 mmol) was added. The reaction mixture was stirred at ambient temperature for 4 h under an inert atmosphere. The progress of the reaction was monitored by TLC (hexane:ethyl acetate 1:1). The solvent was evaporated, the residue was dissolved in ethyl acetate (70 mL) and washed with 5% aqueous NaHCO₃ (40 mL). The ethyl acetate layer was dried over anhydrous Na₂SO₄ and concentrated. Residue obtained was chromatographed (ethyl acetate:hexane, 3:2 as eluent) to give 3 as a foam (0.82 g, 60.3%). R_{f}=0.47 (ethyl acetate:hexane, 3:2). ³¹P NMR (CDCl₃) δ 149.98, 149.57 ppm; MS (API-ES) m/z 743.3 (M−H)

### EXAMPLE 3

### 5′-O-DMT-L-thymidine-3′-O-succinyl CPG (4)

Compound 2 (0.2 g, 0.37 mmol) was mixed with succinic anhydride (0.074 g 0.73 mmol) and DMAP (0.023 g, 0.19 mmol). The mixture was dried over P₂O₅ overnight in vacuum. To this Cl-CH₂-CH₂-Cl (1.1 mL) and triethylamine (0.2 mL, 1.46 mmol) were added. The reaction mixture was heated at 60° C. for 2 h. Diluted the reaction mixture with CH₂Cl₂ (20 mL), washed with 5% aqueous citric acid (20 mL), water (20 mL) and brine (20 mL). The organic phase was dried over anhydrous Na₂SO₄ and concentrated in vacuo (0.22 g, 93%) as a foam. R_{f}=0.23 (5% MeOH in CH₂Cl₂: MeOH). The residue obtained was used as such for the next reaction. ¹H NMR (200 MHz, CDCl₃)δ 1.08 (t, 9H, J=7.18 Hz), 1.4 (s, 3H), 1.92 (m, 2H), 2.54 (s, 6H), 2.62 (m, 10H), 2.91 (m, 1H), 3.37-3.74 (m, 5H), 3.61 (s, 6H), 4.29 (t, 2H, J=4.52 Hz), 5.38 (t, 1H, J=5.48 Hz), 6.05 (d, 1H, J=4.5 Hz), 6.85 (d, 4H, J=8.78 Hz), 7.26-7.42 (m, 9H), 7.62 (s, 1H); ¹³C NMR (50 MHz, CDCl₃) δ 11.33, 28.17, 28.63, 28.89, 37.70, 31.34, 55.03, 63.48, 75.43, 83.62, 84.22, 86.94, 111.36, 113.12, 126.98, 127.81, 127.92, 129.88, 134.98, 135.07, 135.68, 144.03, 150.61, 158.52, 164.54, 171.62, 175.98.

The succinyl derivative (0.19 g 0.25 mmol) was dried over P₂O₅ in vacuo at 40° C. overnight. Anhydrous DMF (0.62 mL) was added followed by 2-(1H-benzotriazole)-1-yl)-1,1,3,3-tetramethyluronium tetrafluoroborate (0.081 g, 0.25 mmol) and N-methylmorpholine (55 μL, 0.5 mmol). Vortexed to give a clear solution. To this anhydrous DMF (2.4 mL) and activated CPG (1.08 g, 115.2 mmol/g, particle size 120/200, mean pore diameter 520 Å) were added. It was then allowed to shake on a shaker for 18 h. Aliquot was withdrawn to estimate the loading capacity. Filtered the functionalized CPG and washed thoroughly with DMF, CH₃CN and Et₂O. Dried in vacuo overnight. Suspended the functionalized CPG (3) in capping solution (2 mL, Cap A, acetic anhydride/lutidine/THF, 2 mL, Cap B, N-methylimidazole/THF, Perspective Biosystems Inc.) and allowed to shake on a shaker for 2 h. Filtered and washed with CH₃CN and Et₂O. Dried in vacuo and loading capacity was determined by standard procedure. Final loading 52.62 μmol/g.

### EXAMPLE 4

### 5′-O-DMT-L-N⁴-benzoyl-2′-deoxyadenosine-3′-O-[(2-cyanoethyl)-N,N-diisopropylphosphoramidite] (5)

1-Chloro-5,3-bis(tolyl)-2-deoxy L-ribose is prepared as described in [Jung, M. E. et. al. Tetrahedron Lett. 1990, 31, 6983-6986; Gosselin, G. et al. Tetrahedron Lett. 1997, 38, 4199-4202, Nucleosides & Nucleotides 1998, 17, 1731-1738]. This is then coupled with N⁴-benzoyl adenine under Vorbruggen condition to give the N⁴-benzoyl-5′,3′-tolyl-1-adenosine. Deprotection of the tolyl group with methylamine gives L-adenosine. It is then converted into N⁴-benzoyl L-adenosine under transient protection conditions in the presence of benzoyl chloride, TMSCl, pyridine and aqueous ammonia. 5′-Tritylation in presence of DMTCl, in pyridine and phosphitylation at the 3′-position gives compound 5.

### EXAMPLE 5

### 5′-O-DMT-L-N⁴-benzoyl-5-methyl-2′-deoxycytidine-3′-O-[(2-cyanoethyl)-N,N-diisopropylphosphoramidite] (6)

Compound 2 is converted into 5′-O-DMT-L-5-methylcytidine according to literature procedure [Divakar K. J. et. al. J. Chem. Soc. Perk. Trans. 1 1982, 1171-1176]. It is then converted into N⁴-benzoyl derivative according to literature procedure [Bhat, V. et. al. Nucleosides Nucleotides 1989, 8, 179-183]. This is then phosphitylated at the 3′-position to give compound 6.

### EXAMPLE 6

### 5′-O-DMT-L-N²-isobutyryl-2′-deoxyguanosine-3′-O-[(2-cyanoethyl)-N,N-diisopropylphosphoramidite] (7)

1-Chloro-5,3-bis(tolyl)-2-deoxy L-ribose is prepared as described in Jung, M. E. et. al. Tetrahedron Lett. 1997, 38, 4199-4202 and Gosselin, G. et. al. Nucleosides & Nucleotides 1998, 17, 1731-1738. This is then coupled with 4-chloro-2-aminopyrrolo [2,3-d]pyrimidine [prepared according to the procedure described by Davoll, J. et. al. J. Chem. Soc. 1960, 131-138] under NaH and acetonitrile [Ramasamy, K. et. al. J. Hetrocyclic Chem., 1988, 25, 1893-1897]. This is then treated with aqueous ammonia at 80° C. to give L-2′-deoxyguanosine. This is converted into L-N²-isobutyryl-2′-deoxyguanosine under transient protection conditions in presence of isobutyryl chloride, pyridine and TMSCl [Ti, G. S. et. al. J. Am. Chem. Soc., 1982, 104, 1316-1319]. This is then converted into 5′-O-DMT-L-N²-isobutyryl-2′-deoxyguanosine in the presence of DMTCl, DMAP and pyridine followed by phosphitylation at 3′-position to give compound 7.

### EXAMPLE 7

### 5′-O-DMT-L-5-(1-propynyl)uridine-3′-O-[(2-cyanoethyl)-N,N-diisopropylphosphamidite] (8)

1-Chloro-5,3-bis(tolyl)-2-deoxy L-ribose is prepared as described in Jung, M. E. et al. Tetrahedron Lett. 1997, 38, 4199-4202 and Gosselin, G. et. al. Nucleosides & Nucleotides 1998, 17, 1731-1738. This is then coupled with 5-iodouracil under Vorbruggen condition to give the L-5-(iodo)-5′,3′-tolyl-1-uridine. This is then coupled with propyne [as described in Switzer C. et. al., Bioorg. Med. Chem. Lett. 1996, 6, 815-818] to give L-5-(propynyl)-5′,3′-tolyl uridine. Deprotection of protecting groups at 5′ and 3′ position gives L-5-(propynyl)uridine. This compound is converted into the 5′-O-DMT compound with DMTCl, DMAP and pyridine followed by phosphitylation to give the title compound 8.

### EXAMPLE 8

### 5′-O-DMT-L-5-(1-propynyl)cytidine-3′-O-[(2-cyanoethyl)-N,N-diisopropylphosphoramidite) (9)

5′-O-DMT-L-5-(1-propynyl)uridine (prepared following the procedure described for compound 7) is converted into 5′-O-DMT-L-5-(1-propynyl)cytidine according to literature procedure [Divakar K. J. et. al. J. Chem. Soc. Perk. Trans. 1 1982, 1171-1176]. This is phosphitylated at the 3′-position to give compound 9.

### EXAMPLE 9

### 5′-O-DMT-L-3(2-deoxy-β-D-erythro-pentofuranosyl)(9I)-1H-pyrimido[5,4-b]benzoxazin-2(3H)-one-3′-O-[(2-cyanoethyl)-N,N-diisopropylphosphoramidite] (10)

L-5-Bromouridine is obtained from 5-bromo uridine and 1-Chloro-5,3-bis(tolyl)-2-deoxy L-ribose under Vorbruggen conditions. This is converted into 5,3-bis(tolyl)-L-3-(2-deoxy-β-D-erythro-pentofuranosyl) (9I)-1H-pyrimido[5,4-b]benzoxazin-2(3H)-one according to literature procedure [Lin, K -Y. et. al. J. Am. Chem. Soc. 1995, 117, 3873-3874, Matteucci, M. D. et. al. 94-US10536]. This is then deprotected with methyl amine, tritylated at 5′ position and phosphitylated at 3′ position to give compound 10.

### EXAMPLE 10

### 5′-O-DMT-L-N⁴-benzoyl-2′-deoxyadenosine-3′-O-succinyl CPG (11)

5′-O-DMT-L-N⁴-benzoyl-2′-deoxyadenosine (prepared as described in the synthesis of compound 5) is converted into 3′-O-succinyl derivative in the presence of succinic anhydride and DMAP in dichloroethane at 60° C. The succinyl derivatives is coupled to amino alkyl CPG in presence of 2-(1H-benzotriazole)-1-yl)-1,1,3,3-tetramethyluronium tetrafluoroborate and N-methylmorpholine in DMF to give compound 11.

### EXAMPLE 11

### 5′-O-DMT-L-N⁴-benzoyl-5-methyl-2′-deoxycytidine-3′-O-succinyl CPG (12)

5′-O-DMT-L-N⁴-benzoyl-5-methyl-2′-deoxycytidine (prepared as described in the synthesis of compound 6) is converted into 3′-O-succinyl derivative in the presence of succinic anhydride and DMAP in dichloroethane at 60° C. The succinyl derivative is coupled to amino alkyl CPG in presence of 2-(1H-benzotriazole)-1-yl)-1,1,3,3-tetramethyluronium tetrafluoroborate and N-methylmorpholine in DMF to yield the compound 12.

### EXAMPLE 12

### 5′-O-DMT-L-N²-isobutyryl-2′-deoxyguanosine-3′-O-succinyl CPG (13)

5′-O-DMT-L-N²-isobutyryl-2′-deoxyguanosine (prepared as described in the synthesis of compound 7) is converted into 3′-O-succinyl derivative in the presence of succinic anhydride and DMAP in dichloroethane at 60° C. The succinyl derivative is coupled to amino alkyl CPG in presence of 2-(1H-benzotriazole)-1-yl)-1,1,3,3-tetramethyluronium tetrafluoroborate and N-methylmorpholine in DMF to yield the compound 13.

### EXAMPLE 13

### 5′-O-DMT-L-5-(1-propynyl)uridine-3′-O-succinyl CPG (14)

5′-O-DMT-L-5-(1-propynyl)uridine (prepared as described in the synthesis of compound 8) is converted into 3′-O-succinyl derivative in the presence of succinic anhydride and DMAP in dichloroethane at 60° C. The succinyl derivative is coupled to amino alkyl CPG in presence of 2-(1H-benzotriazole)-1-yl)-1,1,3,3-tetramethyl-uronium tetrafluoroborate and N-methylmorpholine in DMF to yield the compound 14

### EXAMPLE 14

### 5′-O-DMT-L-5-(1-propynyl)cytidine-3′-O-succinyl CPG (15)

5′-O-DMT-L-5-(1-propynyl)cytidine (prepared as described in the synthesis of compound 8) is converted into 3′-O-succinyl derivative in the presence of succinic anhydride and DMAP in dichloroethane at 60° C. The succinyl derivative is coupled to amino alkyl CPG in the presence of 2-(1H-benzotriazole)-1-yl)-1,1,3,3-tetramethyluronium tetrafluoroborate and N-methylmorpholine in DMF to yield the compound 15.

### EXAMPLE 15

### 5′-O-DMT-L-3(2-deoxy-β-D-erythro-pentofuranosyl)(9I)-1H-pyrimido[5,4-b]benzoxazin-2(3H)-one-3′-O-succinyl CPG (16)

5′-O-DMT-L-3(2-deoxy-β-D-erythro-pentofuranosyl)(9I)-1H-pyrimido[5,4-b]benzoxazin-2(3H)-one (prepared as described in the synthesis of compound 10) is converted into 3′-O-succinyl derivative in the presence of succinic anhydride and DMAP in dichloroethane at 60° C. The succinyl derivative is coupled to amino alkyl CPG in presence of 2-(1H-benzotriazole)-1-yl)-1,1,3,3-tetramethyluronium tetrafluoroborate and N-methylmorpholine in DMF to yield the compound 16.

### EXAMPLE 16

### Synthesis of Oligonucleotides Containing L-thymidine Modification

The amidite 3 was dissolved in anhydrous acetonitrile to give a 0.1 M solution and loaded on to a Expedite Nucleic Acid Synthesis system (Millipore 8909) to synthesize the oligonucleotides. The coupling efficiencies were more than 98%. For the coupling of the modified amidite (3) coupling time was extended to 10 min and this step was carried out twice. All other steps in the protocol supplied by Millipore were used as such. After completion of the synthesis the CPG was suspended in aqueous ammonia (30 wt %) and at room temperature for 2 h to deprotect oligonucleotides form the CPG. Filtered the CPG and heated the filtrate at 55° C. for 6 h to complete the deprotection of all protecting groups. Ammonia was removed on a speed vac concentrator and then the product was purified by High Performance Liquid Chromatography (HPLC, Waters, C-4, 7.8×300 mm, A=50 mM triethylammonium acetate, pH=7, B=acetonitrile, 5 to 60% B in 55 Min, Flow 2.5 mL/min, λ=260 mm). Detritylation with aqueous 80% acetic acid and evaporation followed by desalting by HPLC on Waters C-4 column gave 2′-modified oligonucleotides (Table I). Oligonucleotides were analyzed by HPLC, CGE and mass spectrometry.

### EXAMPLE 17

TABLE I
Oligonucleotides containing L-thymidines

| | | | | HPLC |
|---|---|---|---|---|
| | | | | Retention |
| | | Mass | Mass | Time |
| ISIS No. | Sequence | Calcd | Observed | (min.^{a}) |
| 120745 | 5′ T*GC ATC CCC CAG GCC ACC AT* 3′ | 6591.06 | 6591.29 | 23.40 |
| | (SEQ ID NO: 1) | | | |
| 121785 | 5′ T*C^{o}C^{o}GCGCTGTGATGCA^{o}T^{o}T* 3′ | 6673.02 | 6673.85 | 28.74 |
| | (SEQ ID NO:2) | | | |
| 124585 | 5′ T*C^{o}C^{o}GTCATCGCTC^{o}C^{o}T^{o}C^{o}A^{o}G^{o}G^{o}T* 3′ | 7061.48 | 7061.60 | 33.46 |
| | (SEQ ID NO:3) | | | |

T* = L-Thymidine, All P = S, C^{o} = 2′-O-MOE ^{5Me}C, A^{o} = 2′-O-MOE A, T^{o} = 2′-O-MOE ^{5Me}U, G^{o} = 2′-O- MOE G, ^{a}Waters C-4, 3.9 × 300 mm, solvent A = 50 mm TEAAc,pH 7; Solvent B = CH₃CN; gradient 5-60% B in 55 min; flow rate 1.5 mL/min, λ = 260 nm.

### EXAMPLE 18

TABLE II
Tm values of L-thymidine modified oligonucleotides
against RNA

| | | Tar- | |
|---|---|---|---|
| | | get | |
| | | RNA | ΔTm |
| ISIS # | Sequence | ° C. | ° C. |
| 8651 | TGC ATC CCC CAG GCC ACC AT | 68.7 | |
| | (SEQ ID NO:4) | | |
| 120745 | 5′ T*GC ATC CCC CAG GCC ACC AT* | 66.94 | −1.76 |
| | (SEQ ID NO:5) | | |
| 5132 | 5′ TCCCGCTGTGATGCATT 3′ | 60.6 | |
| | (SEQ ID NO:6) | | |
| 121785 | 5′ T*C^{o}C^{o}CGCTGTGATGCA^{o}T^{o}T* 3′ | 63.3 | 2.7 |
| | (SEQ ID NO:7) | | |

T* = L-Thymidine, All P = S, C^{o} = 2′-O-MOE ^{5Me}C, A^{o} = 2′-O-MOE A, T^{o} = 2′-O-MOE ^{5Me}U, G^{o} = 2′-O- MOE G.

In order to overcome the binding affinity loss due to the L-isomer placement we also incorporated 2′-O-MOE (2′-O-(2-methoxyethyl) modification in the L/D-chimera and evaluated the binding affinity of the resultant chimeric compound to RNA target. The Tm analysis indicated that incorporation of 2′-O-MOE modification along with L-thymidine in the chimera compensates the affinity loss due to L-thymidine towards RNA binding. Thus the designer oligonucleotide construct consisting of combined L-thymidine caps, 2′-O-MOE and 2′-deoxyphophorothioates provide favorable properties for superior antisense oligonucleotide drugs.

### EXAMPLE 19

TABLE III
L-D Chimeric oligonucleotide Gapmers, hemimers and Inverted
Gapmers

| Entry | Sequence | Target | Class |
|---|---|---|---|
| 17 | 5 C*T^{o}A^{o}G^{o}A^{o}TTCCACACTCT^{o}C^{o}G^{o}T^{o}C^{o} 3′ | Mur. MDM2 | Gapmer |
| | (SEQ ID NO:8) | | |
| 18 | 5 C^{o}T^{o}A^{o}G^{o}A^{o}TTCCACACTCT^{o}C^{o}G^{o}T^{o}C* 3′ | Mur. MDM2 | Gapmer |
| | (SEQ ID NO:9) | | |
| 19 | 5′ C*T^{o}A^{o}G^{o}A^{o}TTCCACACTCT^{o}C^{o}G^{o}T^{o}C* 3 | Mur. MDM2 | Gapmer |
| | (SEQ ID NO:10) | | |
| 20 | 5′ CCGGTACCCC^{o}A^{o}G^{o}G^{o}T^{o}TC^{o}T^{o}T^{o}C^{o}A* 3′ | Mur. A-raf | 3′-hemimer |
| | (SEQ ID NO:11) | | |
| 21, 22 | 5′ C*CGGTACCCC^{o}A^{o}G^{o}G^{o}T^{o}TC^{o}T^{o}T^{o}C^{o}A* 3′ | Mur. A-raf | 3′-hemimer |
| | (SEQ ID NO:12) | | |
| 23 | 5′ CTAGATTCC^{o}A^{o}C^{o}A^{o}CTCTCGTC* 3′ | Mur. MDM2 | Inverted gapmer |
| | (SEQ ID NO:13) | | |
| 24 | 5′ C*TAGATTCC^{o}A^{o}C^{o}A^{o}CTCTCGTC 3′ | Mur. MDM2 | Inverted gapmer |
| | (SEQ ID NO:14) | | |
| 25 | 5′C*TAGATTCC^{o}A^{o}C^{o}A^{o}CTCTCGTC 3′ | Mur. MDM2 | Inverted gapmer |
| | (SEQ ID NO:15) | | |

C* = L-Cytidine, A* = L-Adenosine, All P = S, C^{o} = 2′-O-MOE ^{5Me}C, A^{o} = 2′-O-MOE A, T^{o} 2′-O-MOE ^{5Me}U, G^{o} = 2′-O- MOE G.

### EXAMPLE 20

### L-Nucleosides with Novel Nucleobases and Oligonucleotides Derived Therefrom

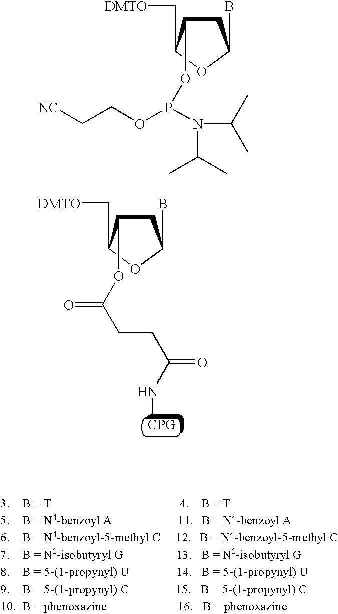

### EXAMPLE 21

### 5′-O-DMT-2′,3′-dideoxy-N⁴-[4-(CPG-succinyl)methylester]benzoylcytidine (29)

2′,3′-dideoxycytidine 26 [Prepared according to the literature procedure Horwitz, J. P. et al. J. Org. Chem., 1967, 32, 817-818] is converted into 5′-O-silyl derivative in presence of TBDMSCl and pyridine. This is then treated with 4-(hydroxymethyl)benzoylchloride in pyridine to give compound 27 (Scheme 3). Compound 27 is treated with succinic anhydride and DMAP in 1,2-dichloroethane to give the succinyl derivative. The succinyl derivative is coupled with aminoalkyl CPG in presence of TBTU and 4-methylmorpholine in DMF to give 28. Compound 28 is desilylated with triethylamine trihydrofluoride and triethylamine in THF. It is then tritylated with DMTCl in pyridine and DMAP to give compound 29.

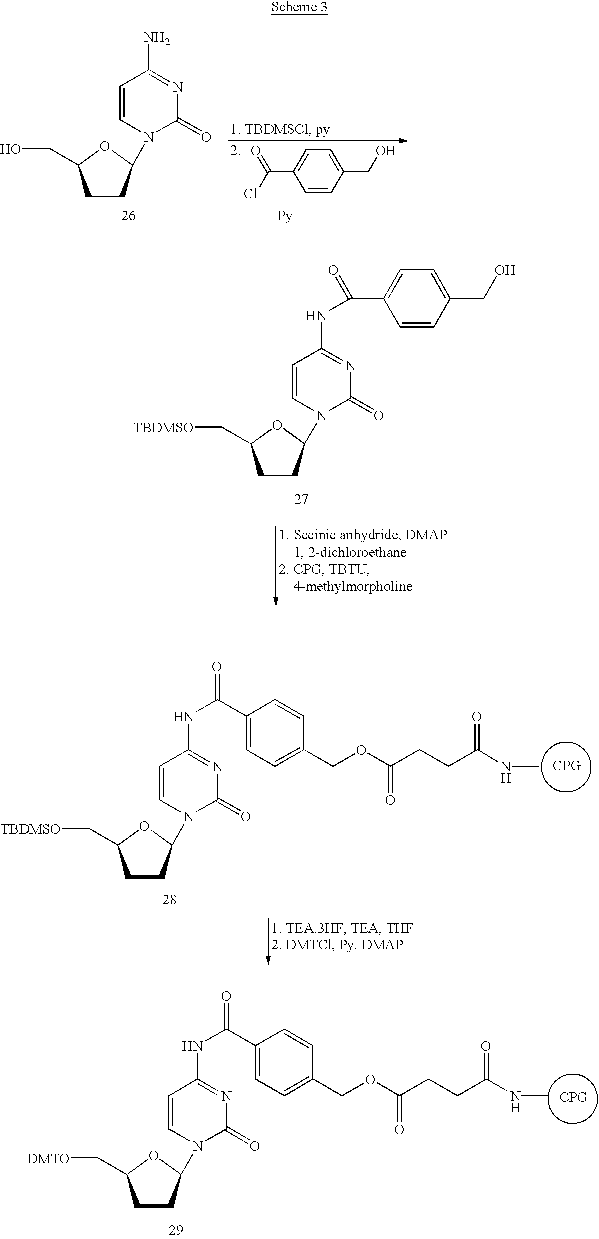

### EXAMPLE 22

### 5′-O-DMT-2′,3′-dideoxy-N⁴-[4-(CPG-succinyl)methylester]benzoyladenosine (33)

2′,3′-Dideoxyadenosine 30 [Prepared according to the literature procedure Horwitz, J. P. et. al. J. Org. Chem. 1967, 32, 817-818] is converted into 5′-O-silyl derivative in presence of TBDMSCl and pyridine. This is then treated with 4-(hydroxymethyl)benzoylchloride in pyridine to give compound 31 (Scheme 4) Compound 31 is treated with succinic anhydride, DMAP in 1,2-dichloroethane to give the succinyl derivative. The succinyl derivative is coupled with aminoalkyl CPG in presence of TBTU and 4-methylmorpholine in DMF to give 32. Compound 32 is desilylated with triethylamine trihydrofluoride and triethylamine in THF. It is then tritylated with DMT chloride to pyridine and DMAP to give compound 33.

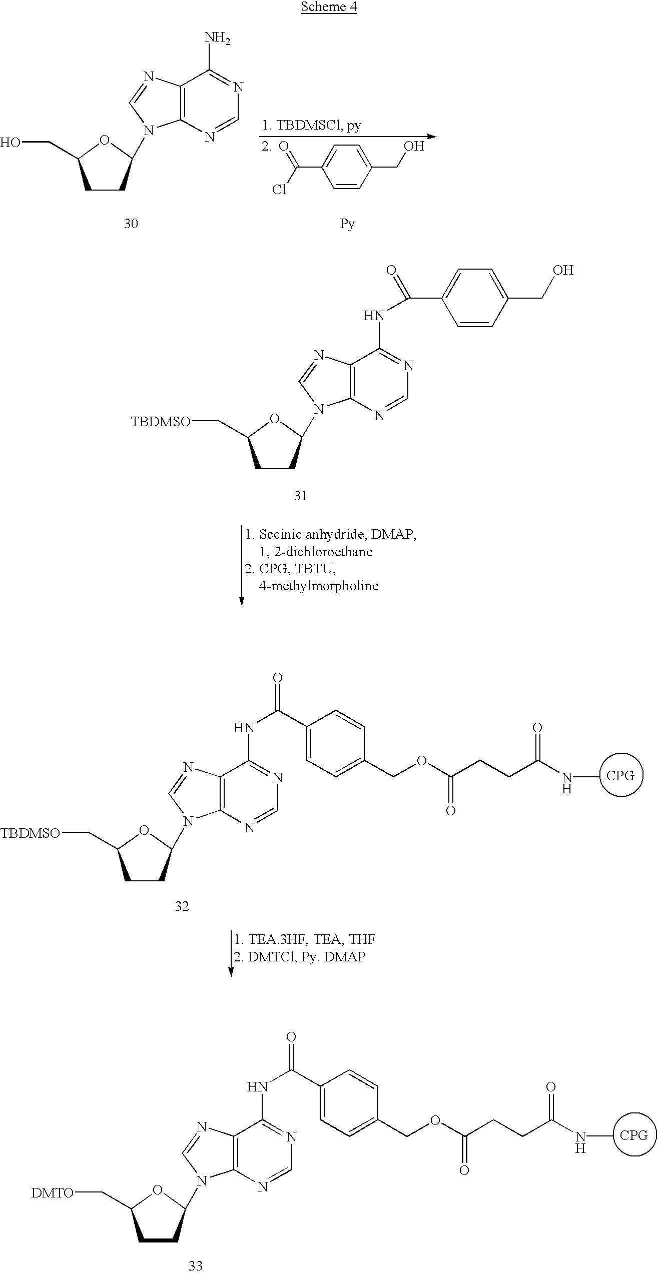

### EXAMPLE 23

### Synthesis of 2′-3′-dideoxy Oligonucleotides

Oligonucleotides 34 (SEQ ID NO:16) and 35 (SEQ ID NO17) are prepared according to the procedure used for the synthesis of compounds 17-25 (SEQ ID NOs:8-15) using solid support 29 and 33 respectively.

### EXAMPLE 24

TABLE IV
2,-3′-Dideoxy containing oligonucleotide Gapmers, hemimers

| Entry | Sequence | Target | Class |
|---|---|---|---|
| 34 | 5 C^{o}T^{o}A^{o}G^{o}A^{o}TTCCACACTCT^{o}C^{o}G^{o}T^{o}C 3′ | Mur. MDM2 | Gapmer |
| | (SEQ ID NO:16) | | |
| 35 | 5′ CCGGTACCCC^{o}A^{o}G^{o}G^{o}T^{o}TC^{o}T^{o}T^{o}C^{o}A* 3′ | Mur. A-raf | 3′-hemimer |
| | (SEQ ID NO: 17) | | |

C* = 2,-3′-Dideoxycytidine, A* = 2,-3′-Dideoxyadenosine All P = S, C^{o} = 2′-O-MOE ^{5Me}C, A^{o} = 2′-O-MOE A, T^{o} = 2′-O-MOE ^{5Me}U, G^{o} = 2′-O- MOE G.

### EXAMPLE 25

### 5′-O-DMT-2′,3′-dideoxy-2′,3′-didehydro-N⁴-[4-CPG-succinyl)methylester]-benzoylcytidine (39)

2′,3′-Dideox-2′,3′-didehydroycytidine 36 [prepared according to the reported procedure, Chu, C. K. et. al. J. Org. Chem. 1989, 54, 217-225] is converted into 5′-O-silyl derivative in presence of TBDMSCl in pyridine. This is then treated with 4-(hydroxymethyl)benzoylchloride in pyridine to give compound 37 (Scheme 5). Compound 37 is treated with succinic anhydride, DMAP in 1,2-dichloroethane to give the succinyl derivative. The succinyl derivative is coupled with aminoalkyl CPG in presence of TBTU and 4-methylmorpholine in DMF to give 38. Compound 38 is desilylated with triethylamine trihydrofluoride and triethylamine in THF. It is then tritylated with DMT chloride in pyridine and DMAP to give compound 39.

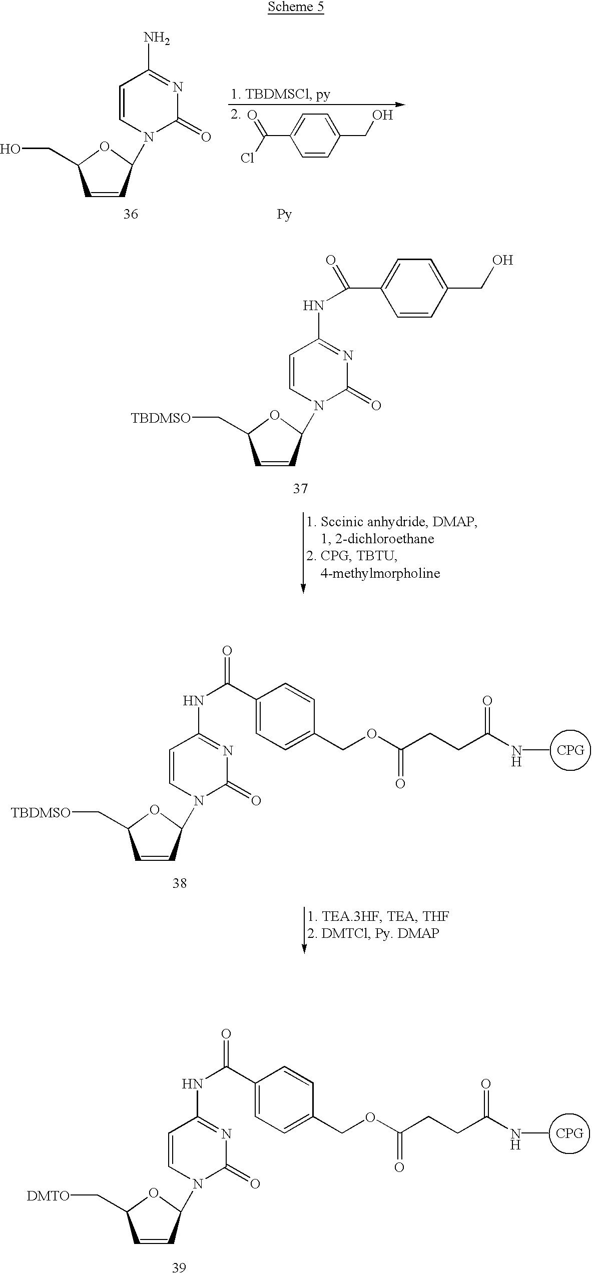

### EXAMPLE 26

### 5′-O-DMT-2′,3′-didehydro-2′,3′-dideoxy-N⁴-[4-(CPG-succinyl)methylester]-benzoyladenosine (43)

2′,3′-Dideoxy-2′,3′-didehydroadenosine 40 [prepared according to the reported procedure, Chu, C. K. et. al. J. Org. Chem. 1989, 54, 217-225] is converted into 5′-O-silyl derivative in presence of TBDMSCl and pyridine. This is then treated with 4-(hydroxymethyl)benzoylchloride in pyridine to give compound 41 (Scheme 6). Compound 41 is treated with succinic anhydride, DMAP in 1,2-dichloroethane to give the succinyl derivative. The succinyl derivative is coupled with aminoalkyl CPG in the presence of TBTU and 4-methylmorpholine in DMF to give 42. Compound 42 is desilylated with triethylamine trihydrofluoride and triethylamine in THF. It is then tritylated with DMTCl in pyridine and DMAP to give compound 43.

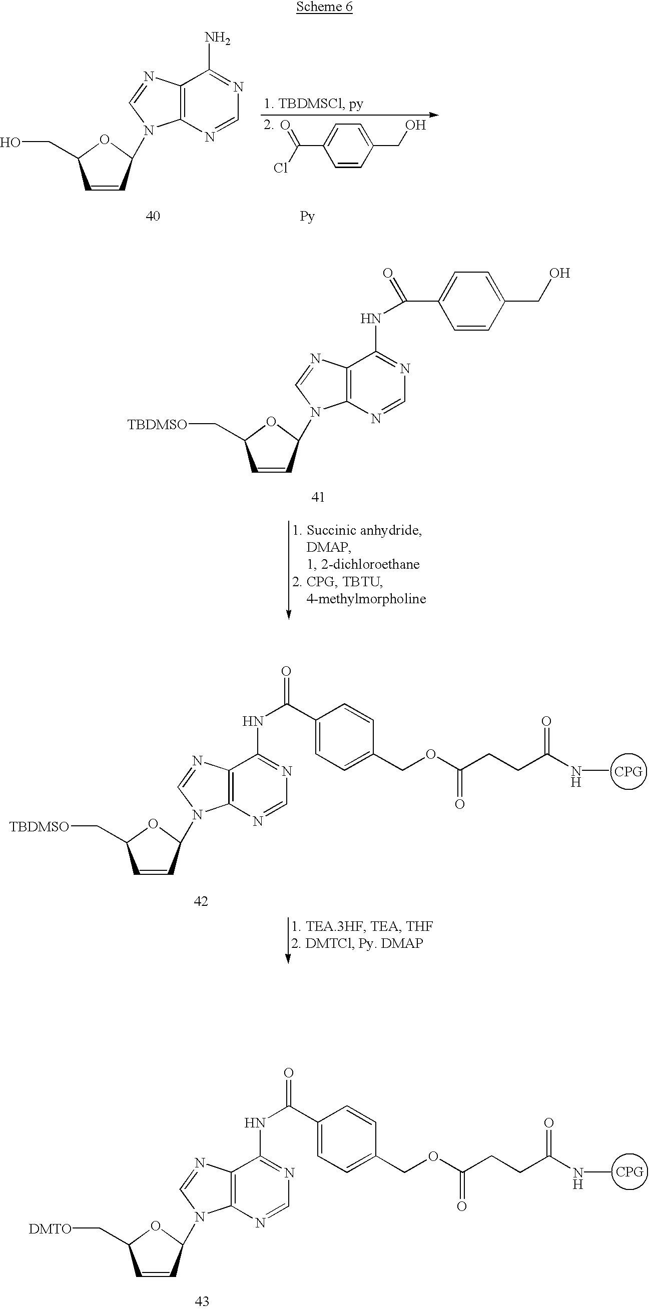

### EXAMPLE 27

TABLE V
2,3′-Didehydro-2′,3′-dideoxy modified nucleoside containing
Chimeric oligonucleotide Gapmers, hemimers

| Entry | Sequence | Target | Class |
|---|---|---|---|
| 44 | 5 C^{o}T^{o}A^{o}G^{o}A^{o}TTCCACACTCT^{o}C^{o}G^{o}T^{o}C* 3′ | Mur. MDM2 | Gapmer |
| | (SEQ ID NO:18) | | |
| 45 | 5′ CCGGTACCCC^{o}A^{o}G^{o}G^{o}T^{o}T^{o}C^{o}T^{o}T^{o}C^{o}A* 3′ | Mum A-raf | 3′-hemimer |
| | (SEQ ID NO:19) | | |

C* = 2,3′-Didehydro-2′,3′-dideoxycytidine, A* = 2,3′-Didehydro-2′,3′-dideoxyadenosine, All P = S. C^{o} = 2′-O-MOE ^{5Me}C, A^{o} = 2′-O-MOE A, T^{o} = 2′-O-MOE ^{5Me}U G^{o} = 2′-O- MOE G.

### EXAMPLE 28

### 5′-O-DMT-2′,3′-dideoxy-2′-fluoro-N⁴-[4-(CPG-succinyl)methylester]benzoyl-cytidine (50)

2′,3′-Dideoxy-2′-fluro uridine 46 [prepared as reported, Martin J. A. et. al. J. Med. Chem. 1990, 33, 2137-2145] as converted into 2′,3′-dideoxy-2′-flurocytidine 47 (Scheme 7) according to the reported procedure [Reference:- Divakar, K. J. et. al. J. Chem. Soc. Perk. Trans. 1 1982, 1171-1176]. Compound 47 is converted into 5′-O-silyl derivative in presence of TBDMSCl and pyridine. This is then treated with 4-(hydroxymethyl)benzoylchloride in pyridine to give compound 48. Compound 48 is treated with succinic anhydride, DMAP in 1,2-dichloroethane to give the succinyl derivative. The succinyl derivative is coupled with aminoalkyl CPG in presence of TBTU and 4-methylmorpholine in DMF to give 49. Compound 49 is desilylated with triethylamine trihydrofluoride and triethylamine in THF. It is then tritylated with DMTCl in pyridine and DMAP to give compound 50.

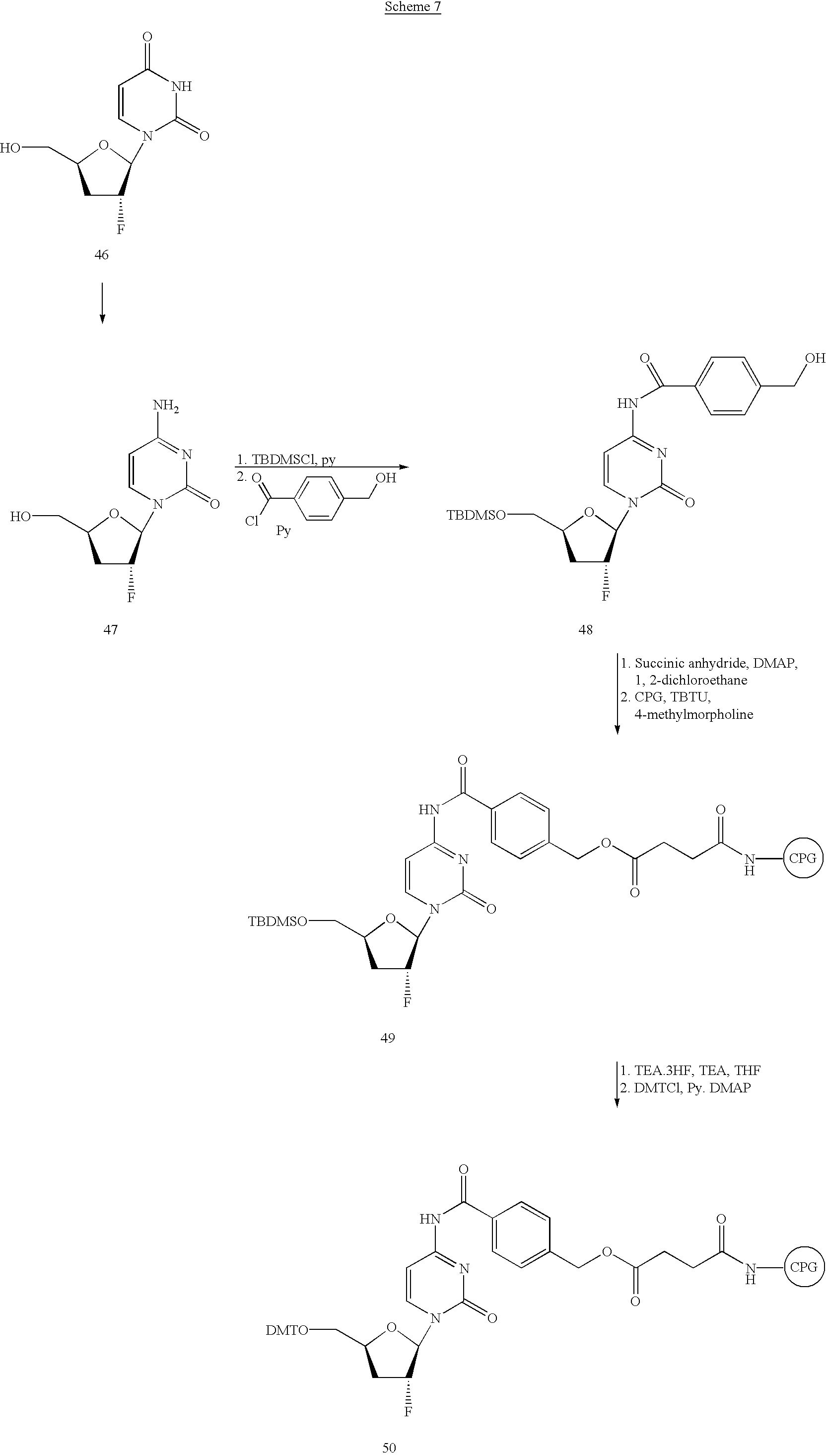

### EXAMPLE 29

TABLE VI
3′-Deoxy-2′-fluorocytidine Chimeric oligo-
nucleotide Gapmers, hemimers and Inverted Gapmers

| En- | | Tar- | |
|---|---|---|---|
| try | Sequence | get | Class |
| 51 | 5 C^{o}T^{o}A^{o}G^{o}A^{o}TTCCACACTCT^{o}C^{o}G^{o}T^{o}C* 3′ | Mur. | Gapmer |
| | (SEQ ID NO:20) | MDM2 | |

C* = 2′,3′-Dideoxy-2′-fluorocytidine, All P = S, C^{o} = 2′-O-MOE ^{5Me}C, A^{o} = 2′-O-MOE A, T^{o} = 2′-O-MOE ^{5Me}U, G^{o} = 2′-O- MOE G.

### EXAMPLE 30

### 5′-O-DMT-2′,3′-deoxy-3′-fluro-N⁴-[4-(CPG-succinyl)methylester]benzoyl-cytidine (56)

2′,3′-Dideoxy-3′-fluro uridine 52 [prepared according to thereported procedure Zaitseva, G. V. et. al. Bioorg. Khim. 1988, 14, 1275-1281] is converted into 2′,3′-dideoxy-3′-fluorocytidine 53 (Scheme 8) according to the reported procedure [Reference:-.Divakar, K. J. et. al. J. Chem. Soc. Perk. Trans. 1 1982, 1171-1176]. Compound 53 is converted into 5′-O-silyl derivative in presence of TBDMSCl and pyridine. This is then treated with 4-(hydroxymethyl)benzoyl chloride in pyridine to give compound 54. Compound 54 is treated with succinic anhydride, DMAP in 1,2-dichloroethane to give the succinyl derivative. The succinyl derivative is coupled with aminoalkyl CPG in presence of TBTU and 4-methylmorpholine in DMF to give 55. Compound 55 is desilylated with triethylamine trihydrofluoride and triethylamine in THF. It is then tritylated with DMT chloride in pyridine and DMAP to give compound 56.

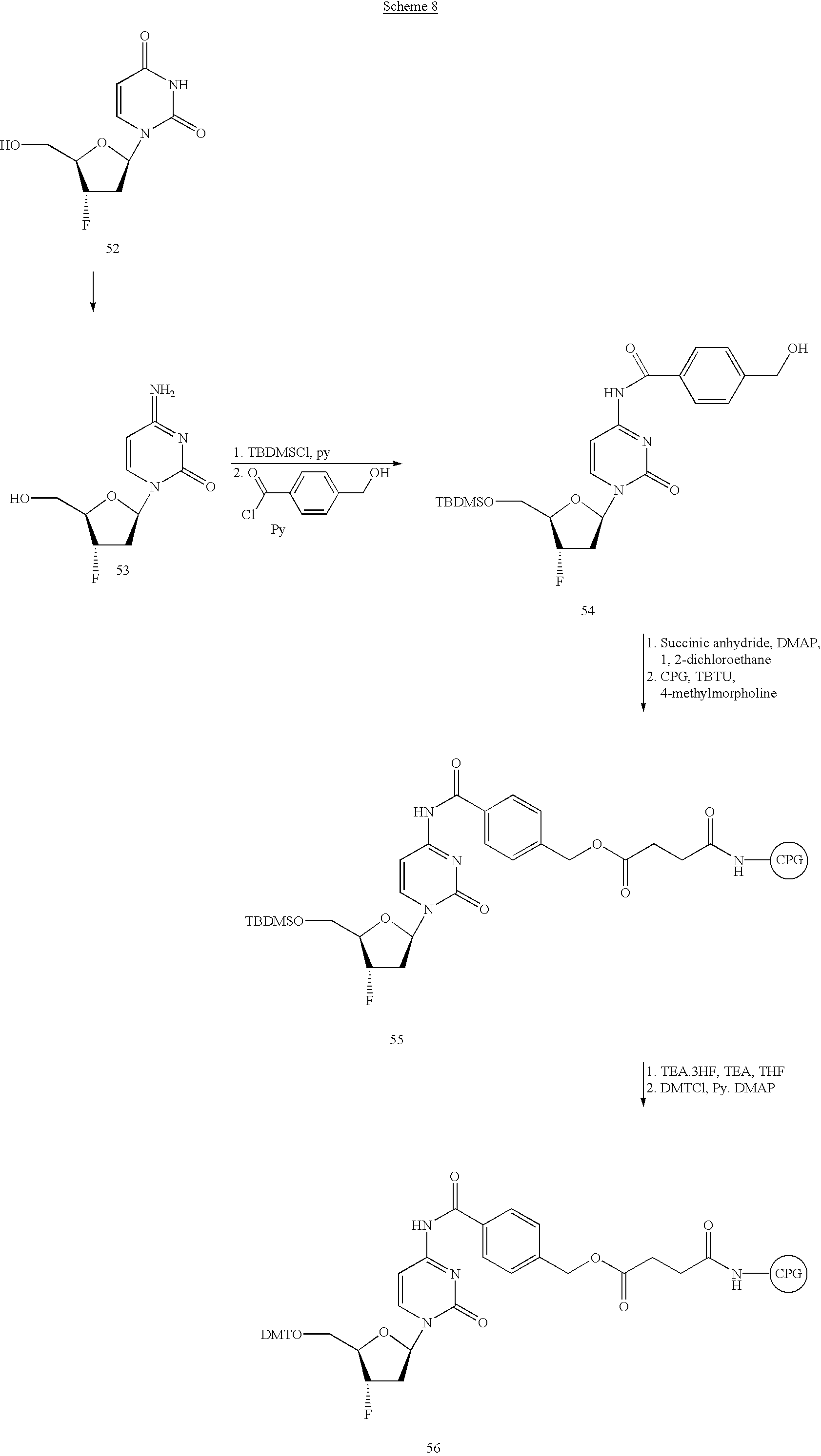

### EXAMPLE 31

TABLE VII
2′,3′-Dideoxy-3′-fluorocytidine Chimeric oligo-
nucleotide Gapmers

| En- | | Tar- | |
|---|---|---|---|
| try | Sequence | get | Class |
| 57 | 5 C^{o}T^{o}A^{o}G^{o}A^{o}TTCCACACTCT^{o}C^{o}G^{o}T^{o}C* 3′ | Mur. | Gapmer |
| | (SEQ ID NO:20) | MDM2 | |

C* = 2′,3′-Dideoxy-3′-fluorocytidine, All P = S, C^{o} = 2′-O-MOE ^{5Me}C, A^{o} =2′-O-MOE A, T^{o} = 2′-O-MOE ^{5Me}U, G^{o} = 2′-O- MOE G.

### EXAMPLE 32

### 5′-O-DMT-3′-deoxy-2′-O-[2-(methoxy)ethyl]-N⁴-[4-(CPG-succinyl)methyl-ester]benzoylcytidine (62)

5′-O-TBDMS-N⁴-benzoyl-5-methylcytidine 58 is synthesized according to the literature procedure [Reese, C. B. et. al. Tetrahedron Lett. 1999, 55, 5635-5640]. The compound 58 is then converted into 59 according to reported procedure [Danel, K. et. al. J. Med. Chem. 1996, 39, 2427-2431]. Compound 59 is converted into 5′-O-silyl derivative in presence of TBDMSCl and pyridine. This is then treated with 4-(hydroxymethyl)benzoylchloride in pyridine to give compound 60. Compound 60 is treated with succinic anhydride, DMAP in 1,2-dichloroethane to give the succinyl derivative. The succinyl derivative is coupled with aminoalkyl CPG in presence of TBTU and 4-methylmorpholine in DMF to give 61. Compound 61 is desilylated with triethylamine trihydrofluoride and triethylamine in THF. It is then tritylated with DMTCl in pyridine and DMAP to give compound 62.

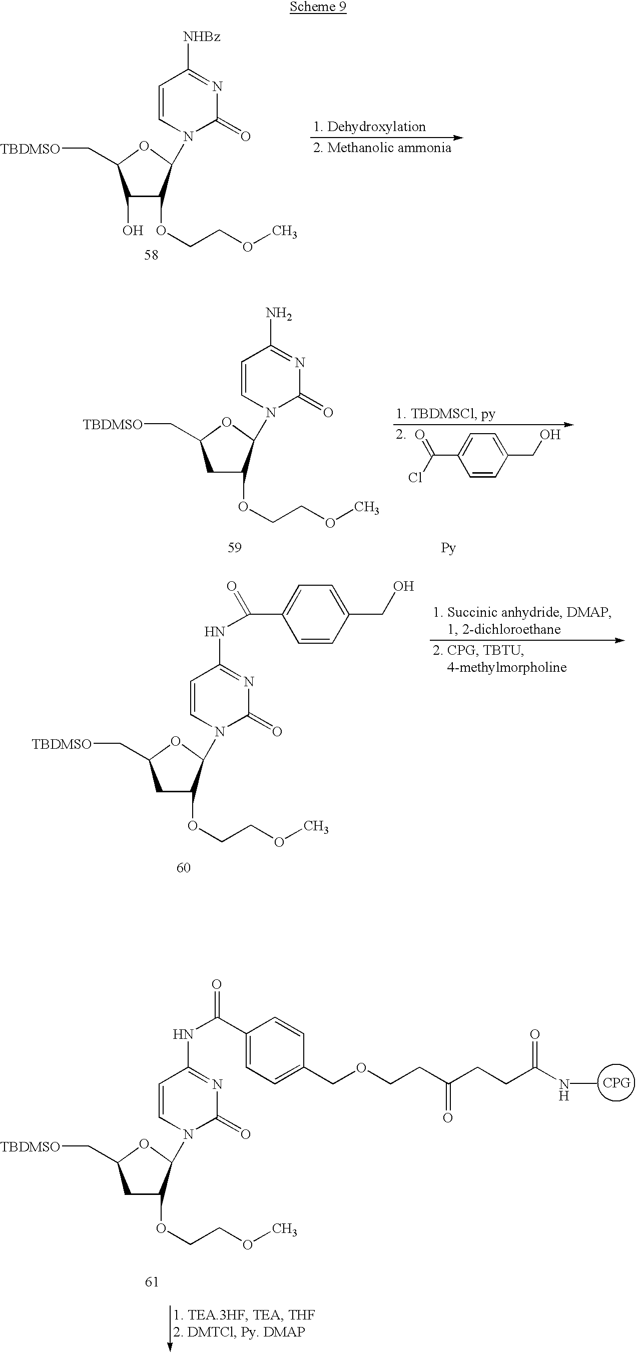

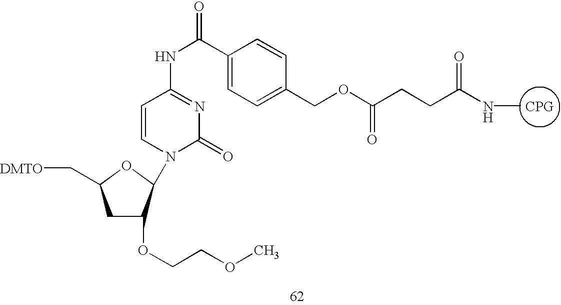

### EXAMPLE 33

TABLE VIII
3′-Deoxy-2′-O-[2-(methoxy)ethyl]-5-methylcytidine
Chimeric oligonucleotide Gapmers

| En- | | Tar- | |
|---|---|---|---|
| try | Sequence | get | Class |
| 63 | 5 C^{o}T^{o}A^{o}G^{o}A^{o}TTCCACACTCT^{o}C^{o}G^{o}T^{o}C* 3′ | Mur. | Gapmer |
| | (SEQ ID NO:21) | MDM2 | |

C* = 3′-Deoxy-2′-O-[2-(methoxy)ethyl]-5-methylcytidine, All P = S, C^{o} = 2′-O-MOE ^{5MeC, A}^{o} = 2′-O-MOE A, T^{o} = 2′-O-MOE ^{5Me}U, G^{o} = 2′-O- MOE G.

### EXAMPLE 34

### N-trifluroacetyl-pyrrolidine-2-(DMT)methanol-3-O-[(2-cyanoethyl)-N,N-diisopropylphosphoramidite] (67)

Compound 64 is synthesized according to the literature procedure [Huwe, C. M. et. al. Synthesis, 1997, 1, 61-67]. It is then converted into trifluromethyl derivative 65 in presence of ethyl trifluroacetate in ethanol. Compound 65 is tritylated to give compound 66. Compound 66 is phosphitylated to give the compound 67.

### EXAMPLE 35

### 3-O-(CPG-succinyl)-N-trifluoroacetyl-pyrrolidine-2-(DMT)methanol (68)

Compound 66 is treated with succinic anhydride, DMAP in 1,2-dichloroethane to give the succinyl derivative. The succinyl derivative is coupled with aminoalkyl CPG in presence of TBTU and 4-methylmorpholine in DMF to give 68.

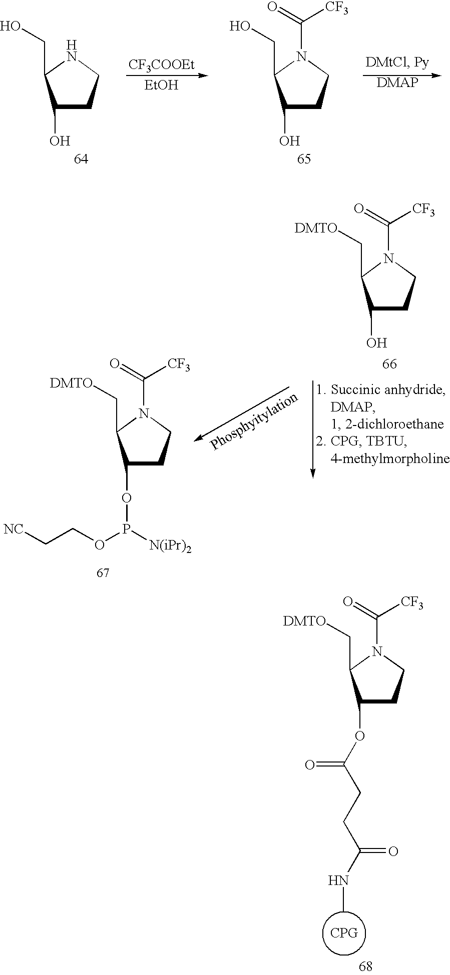

### EXAMPLE 36

TABLE IX
3-hydroxy-2-pyrrolidinemethanol Chimeric oligonucleotide Gapmers,
hemimers

| Entry | Sequence | Target | Class |
|---|---|---|---|
| 69 | 5 C^{o}T^{o}A^{o}G^{o}A^{o}TTCCACACTCT^{o}C^{o}G^{o}T^{o}B* 3′ | Mur. MDM2 | Gapmer |
| | (SEQ ID NO:22) | | |
| 70 | 5 B*C^{o}T^{o}A^{o}G^{o}A^{o}TTCCACACTCT^{o}C^{o}G^{o}T^{o}B* 3′ | Mur. MDM2 | Gapmer |
| | (SEQ ID NO:23) | | |
| 71 | 5′ CCGGTACCCC^{o}A^{o}G^{o}G^{o}T^{o}T^{o}C^{o}T^{o}T^{o}C^{o}AB* 3′ | Mur. A-raf | 3′-hemimer |
| | (SEQ ID NO:24) | | |
| 72 | 5′ B*CCGGTACCCC^{o}A^{o}G^{o}G^{o}T^{o}T^{o}C^{o}T^{o}T^{o}C^{o}AB* 3′ | Mur. A-raf | 3′-hemimer |
| | (SEQ ID NO:25) | | |

B* = 3-hydroxy-2-pyrrolidinemethanol, All P = S, C^{o} = 2′-O-MOE ^{5Me}C, A^{o} = 2′-O-MOE A, T^{o} = 2′-O-MOE ^{5Me}U, G^{o} = 2′-O- MOE G.

### EXAMPLE 37

### 1-[2-(O-succinylCPG)-1-[2-hydroxy-1-(O-DMT-methyl)ethoxy]ethyl]cytosine (76)

Compound 73 is prepared according to the reported procedure (Scheme 11) [Reference:-Bessodes, M. et. al. Tetrahedron Lett. 1985, 26(10), 1305-1306]. This is converted into silylated compound in presence of 1,3-dichloro-1,1,3,3-tetraisopropyldisiloxane in pyridine followed by benzoylation of exocyclic amino group with benzoic anhydride in DMF give compound 74. Compound 74 is succinylated to give sucinyl derivative. The succinyl derivative is coupled with aminoalkyl CPG in presence of TBTU and 4-methylmorpholine in DMF to give 75. This is desilylated and tritylated to give compound 76.

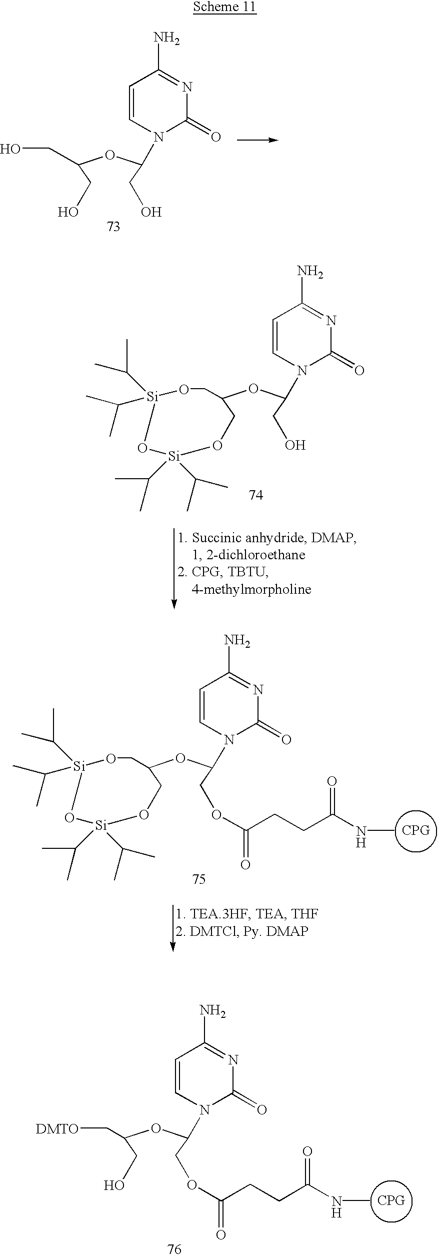

### EXAMPLE 38

### 1-[2-O-(acetyl)-1-[2-[(2-cyanoethyl)-N,N-diisopropylphosphoramidite]-1-(O-DMT-methyl)ethoxy]ethyl]cytosine (79)

Compound 73 is silylated with 1,3-dichloro-1,1,3,3-tetraisopropyldisiloxane in pyridine to give compound 77. This is then acetylated with acetyl chloride in pyridine to give compound 78. Compound 78 is desilylated with TEA, 3HF and TEA and THF. This is tritylated with DMTCl, DMAP and pyridine followed by phosphitylation give compound 79.

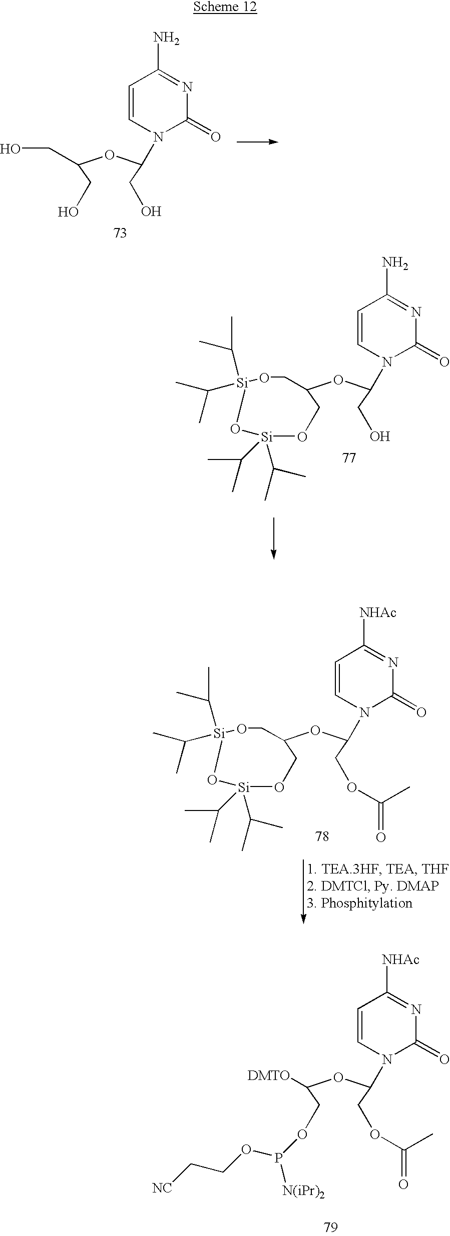

TABLE X
1-[2-hydroxy-1-[2-hydroxy-1-(hydroxymethyl)ethoxy]
ethylcytosine Chimeric oligonucleotide Gapmers

| En- | | Tar- | |
|---|---|---|---|
| try | Sequence | get | Class |
| 80 | 5 C^{o}T^{o}A^{o}G^{o}A^{o}TTCCACACTCT^{o}C^{o}G^{o}T^{o}C* 3′ | Mur. | Gapmer |
| | (SEQ ID NO:26) | MDM2 | |
| 81 | 5 C*T^{o}A^{o}G^{o}A^{o}TTCCACACTCT^{o}C^{o}G^{o}T^{o}C* 3′ | Mur. | Gapmer |
| | (SEQ ID NO:27) | MDM2 | |

C* = 1-[2-hydroxy-1-[2-hydroxy-1-(hydroxymethyl)ethoxy]ethylcytosine, All P = S, C^{o} = 2′-O-MOE ^{5Me}C, A^{o} = 2′-O-MOE A, T^{o} =2′-O-MOE ^{5Me}U, G^{o} = MOE G.

### EXAMPLE 40

### 1-[2-O-(acetyl)-1-[2-(2-cyanoethyl)-N,N-diisopropylphosphoramidite]-1-(O-DMT-methyl)thioethyl]ethyl]cytosine (86)

Compound 82 is synthesized according to literature procedure [Nake, T. et. al. J. Am. Chem. Soc. 2000, 122, 7233-7243]. This is converted into 83 by following a reported procedure for cleavage of vicinlal diols and subsequent reduction of aldehyde thus obtained [Bessodes, M. et. al. Tetrahedron Lett. 1985, 26(10), 1305-1306]. Compound 83 is silylated with 1,3-dichloro-1,1,3,3-tetraisopropyldisiloxane in pyridine to give compound 84. This is then acetylated with acetyl chloride in pyridine to give compound 85. Compound 85 is desilylated with TEA.3HF and TEA in THF. This is tritylated with DMTCl, DMAP and pyridine followed by phosphitylation give compound 86.

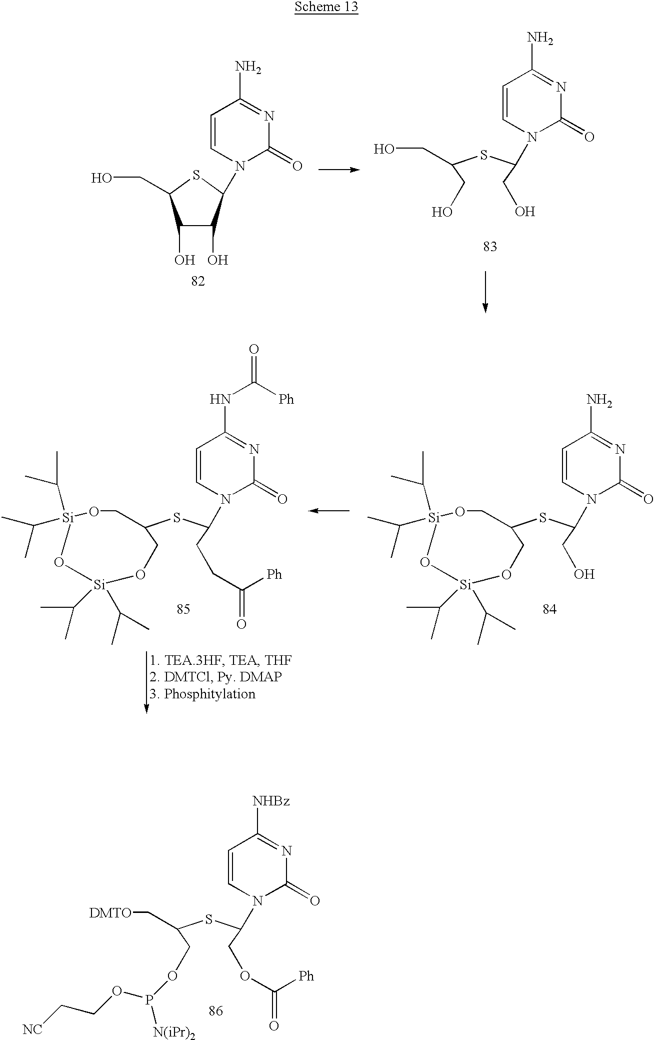

### EXAMPLE 41

### 1-[2-(O-succinyl-CPG)-1-[2-hydroxy-1-(O-DMT-methyl)thioethyl]ethyl]cytosine (89)

Compound 83 is converted into silylated compound in presence of 1,3-dichloro-1,1,3,3-tetraisopropyldisiloxane in pyridine followed by benzoylation of exocyclic amino group with benzoic anhydride in DMF give compound 87. Compound 87 is succinylated to give sucinyl derivative. The succinyl derivative is coupled with aminoalkyl CPG in presence of TBTU and 4-methylmorpholine in DMF to give 88. This is desilylated followed by tritylation give compound 89.

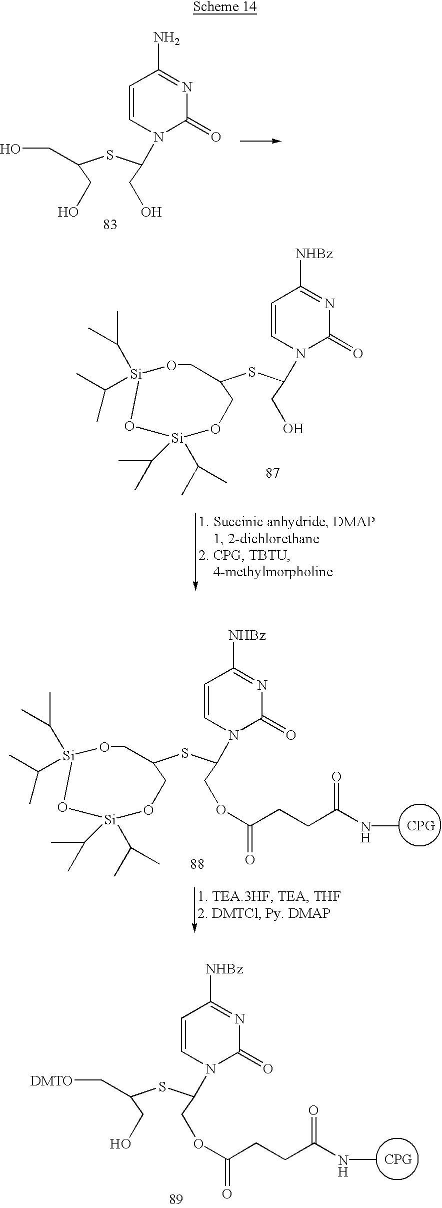

### EXAMPLE 42

TABLE XI

1-[2-hydroxy-1-[2-hydroxy-1-(hydroxymethyl)thioethyl]ethylcytosine
Chimeric oligonucleotide Gapmers

| | | | |
|---|---|---|---|
| Entry | Sequence | Target | Class |

| | | | | |
|---|---|---|---|---|
| 90 | 5 C⁰T⁰A⁰G⁰A⁰TTCCACACTCT⁰C⁰G⁰T⁰C* 3′ | (SEQ ID NO:28) | Mur. MDM2 | Gapmer |
| 91 | 5 C*T⁰A⁰G⁰A⁰TTCCACACTCT⁰C⁰G⁰T⁰C* 3′ | (SEQ ID NO:29) | Mur. MDM2 | Gapmer |

C* = 1-[2-hydroxy-1-[2-hydroxy-1-(hydroxymethyl)thioethyl]ethylcytosine,
All P = S,
C⁰ = 2′-O-MOE ^{5Me}C,
A⁰ = 2′-O-MOE A,
T⁰ = 2′-O-MOE ^{5Me}U,
G⁰ = 2′-O-MOE G.

### EXAMPLE 43

### 5′-O-DMT-2′,3′-dideoxy-3′-(N-acetyl)amino-N⁴-[4-(CPG-succinyl)methylester]benzoylcytidine (95)

Compound 92 is prepared according to the procedure reported in the literature (Reference:-Krenitsky, T. A. et. al. J. Med. Chem. 1983, 26(6), 891-895). This is then selectively tritylated with DMTCl and pyridine to give the 5′-O-DMT derivative which is acetylated to give acetylated product. Selective removal of the acetyl group at N⁴-position with aqueous ammonia at room temperature gives compound 93. This is then treated with 4-(hydroxymethyl)benzoyl chloride in pyridine to give compound 94. Compound 94 is treated with succinic anhydride, DMAP in 1,2-dichloroethane to give the succinyl derivative. The succinyl derivative is coupled with aminoalkyl CPG in presence of TBTU and 4-methylmorpholine in DMF to give 95.

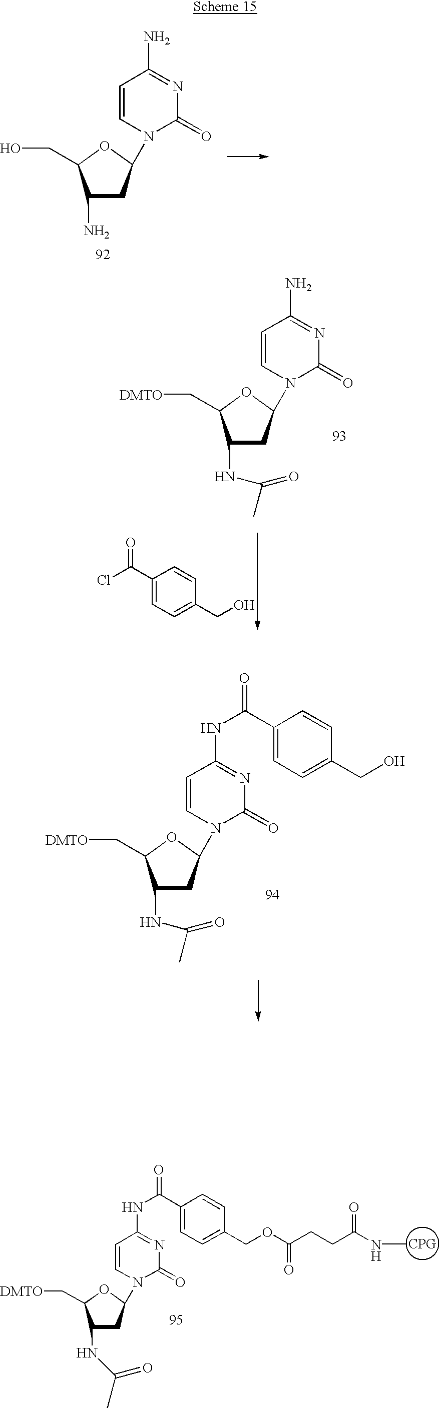

### EXAMPLE 44

TABLE XII

2′,3′-dideoxy-3′-(amino)cytidine Chimeric oligonucleotide Gapmers

| | | | |
|---|---|---|---|
| Entry | Sequence | Target | Class |

| | | | | |
|---|---|---|---|---|
| 96 | 5 C⁰T⁰A⁰G⁰A⁰TTCCACACTCT⁰C⁰G⁰T⁰C* 3′ | (SEQ ID NO:30) | Mur. MDM2 | Gapmer |

C* = 2′,3′-dideoxy-3′-(amino)cytidine,
All P = S,
C⁰ = 2′-O-MOE ^{5Me}C,
A⁰ = 2′-O-MOE A,
T⁰ = 2′-O-MOE ^{5Me}U,
G⁰ = 2′-O-2′-O-MOE G.

### EXAMPLE 45

### 5′-O-DMT-2′-deoxy-3′-S-phenyl-3′-thio-N⁴-[4-(CPG-succinyl)methylester]-benzoylcytidine (101)

2′-Deoxy-3′-S-phenyl-3′-thiouridine 97 [prepared as reported in Kawakami, H. et. al. Heterocycles, 1991, 32(12), 2451-2470] is converted into 2′-deoxy-3-S-phenyl-3-thiocytidine 98 (Scheme 7) according to the reported procedure [Divakar, K. J. et. al. J. Chem. Soc. Perk. Trans. 1, 1982, 1171-1176]. Compound 98 is converted into 5′-O-silyl derivative in presence of TBDMSCl and pyridine. This is then treated with 4-(hydroxymethyl)benzoylchloride in pyridine to give compound 99. Compound 99 is treated with succinic anhydride, DMAP in 1,2-dichloroethane to give the succinyl derivative. The succinyl derivative is coupled with aminoalkyl CPG in presence of TBTU and 4-methylmorpholine in DMF to give 100. Compound 100 is desilylated with triethylamine trihydrofluoride and triethylamine in THF. It is then tritylated with DMTCl in pyridine and DMAP to give compound 101.

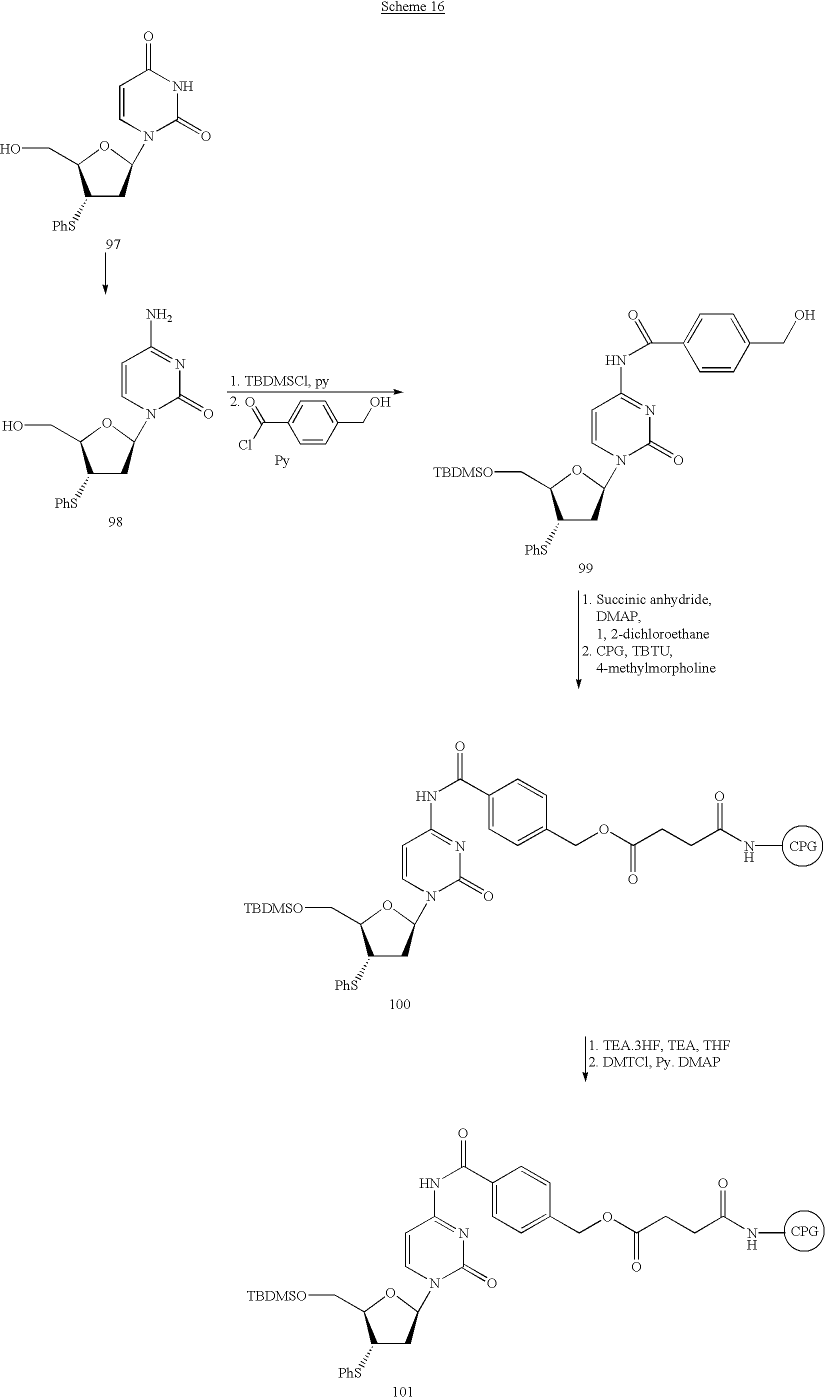

### EXAMPLE 46

TABLE XIII
2′-deoxy-3′-S-phenyl-3′-thiocytidine Chimeric oligonucleotide Gapmers

| Entry | Sequence | | Target | Class |
|---|---|---|---|---|
| 102 | 5 C⁰T⁰A⁰G⁰A⁰TTCCACACTCT⁰C⁰G⁰T⁰C* 3 | (SEQ ID NO:31) | Mur. MDM2 | Gapmer |

C* = 2′-deoxy-3′-S-phenyl-3′-thiocytidine,
All P = S,
C⁰ = 2′-O-MOE ^{5Me}C
A⁰ = 2′-O-MOE A,
T⁰ = 2′-O-MOE ^{5Me}U,
G⁰ = 2′-O-2′-O-MOE G.

### EXAMPLE 47

### 5′-O-DMT-3′-deoxy-2′-S-phenyl-2′-thio-N⁴-[4-(CPG-succinyl)methylester]-benzoylcytidine (107)

3′-Deoxy-2′-S-phenyl-2′-thiouridine 103 [prepared as reported, Kawakami, H. et. al. Heterocycles, 1991, 32(12), 2451-2470] is converted into 2′,3′-dideoxy-2′-flurocytidine 104 (Scheme 17) according to the reported procedure [Divakar, K. J. et. al., J. Chem. Soc. Perk. Trans. 1 1982, 1171-1176]. Compound 104 is converted into 5′-O-silyl derivative in presence of TBDMSCl and pyridine. This is then treated with 4-(hydroxymethyl)benzoylchloride in pyridine to give compound 105. Compound 105 is treated with succinic anhydride, DMAP in 1,2-dichloroethane to give the succinyl derivative. The succinyl derivative is coupled with aminoalkyl CPG in presence of TBTU and 4-methylmorpholine in DMF to give 106. Compound 106 is desilylated with triethylamine trihydrofluoride and triethylamine in THF. It is then tritylated with DMTCl in pyridine and DMAP to give compound 107.

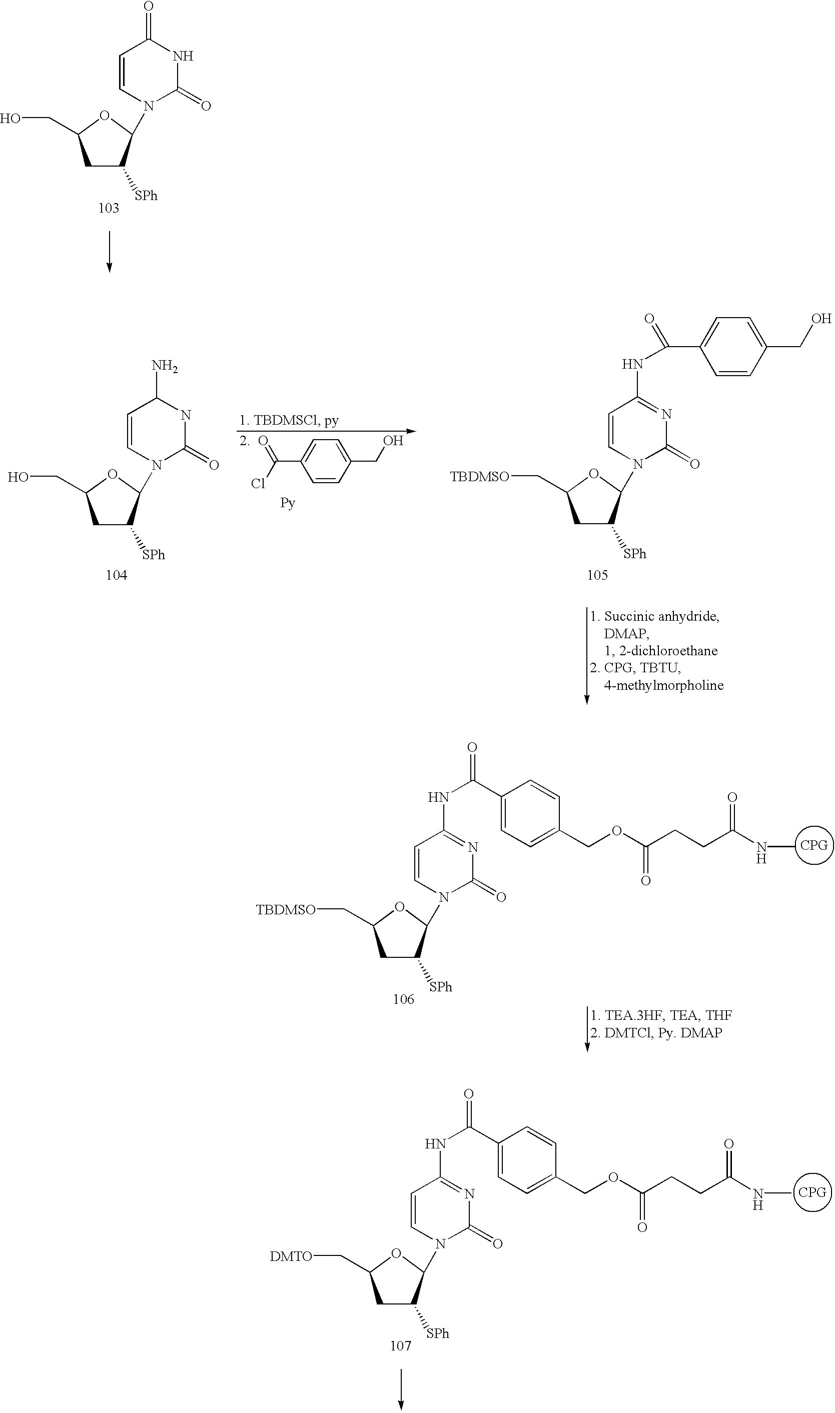

### EXAMPLE 48

TABLE XIV

3′-deoxy-2′-S-phenyl-2′-thiocytidine Chimeric oligonucleotide Gapmers

| Entry | Sequence | | Target | Class |
|---|---|---|---|---|
| 108 | 5 C⁰T⁰A⁰G⁰A⁰TTCCACACTCT⁰C⁰G⁰T⁰C* 3′ | (SEQ ID NO:32) | Mur. MDM2 | Gapmer |

C* = 3′-deoxy-2′-S-phenyl-2′-thiocytidine,
All P = S,
C⁰ = 2′-O-MOE ^{5Me}C,
A⁰ = 2′-O-MOE A,
T⁰ = 2′-O-MOE ^{5Me}U,
G⁰ = 2′-O- 2′-O-MOE G.

### EXAMPLE 49

### 5′-O-DMT-1[2,3-deoxy-2-N-morpholino-β-D-glycero-pent-2-enofuranosyl]-cytosine-N⁴-[4-(CPG-succinyl)methylester]benzoyl (113)

1[2,3-Deoxy-2-N-morpholino-β-D-glycero-pent-2-enofuranosyl]uracil 109 [prepared as reported in Kandasamy, S. et. al. Tetrahedron, 1996, 52(13), 4877-4882] is converted into 2′,3′-dideoxy-2′-flurocytidine 100 (Scheme 18) according to the reported procedure [Divakar, K. J. et. al. J. Chem. Soc. Perk. Trans. 1 1982, 1171-1176]. Compound 110 is converted into 5′-O-silyl derivative in present of TBDMSCl and pyridine. This is then treated with 4-(hydroxymethyl)benzoylchloride in pyridine to give compound 111. Compound 111 is treated with succinic anhydride, DMAP in 1,2-dichloroethane to give the succinyl derivative. The succinyl derivative is coupled with aminoalkyl CPG in presence of TBTU and 4-methylmorpholine in DMF to give 112. Compound 112 is desilylated with triethylamine trihydrofluoride and triethylamine in THF. It is then tritylated with DMT chloride in pyridine and DMAP to give compound 113.

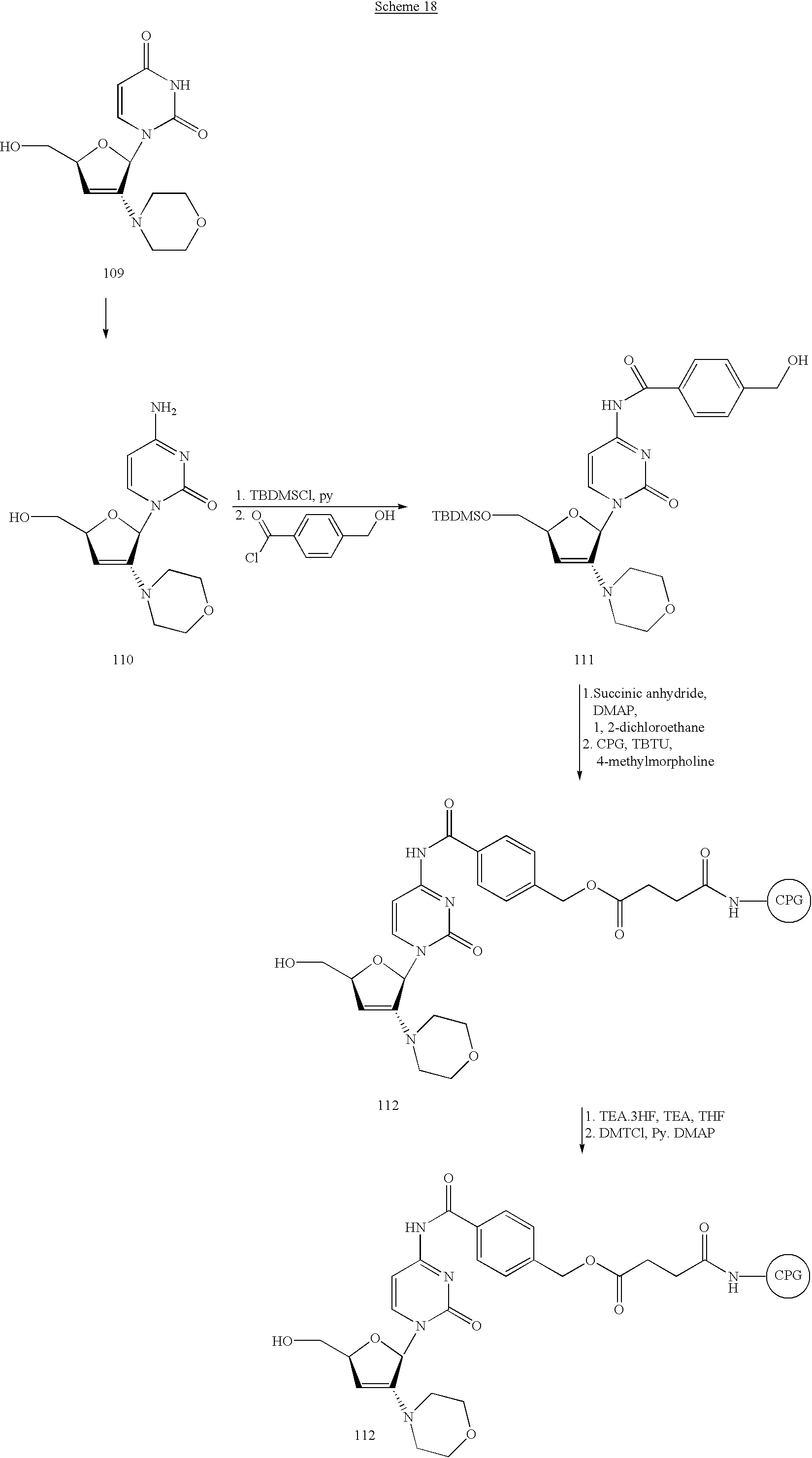

### EXAMPLE 50

TABLE XV

1[2,3-deoxy-2-N-morpholino-β-D-glycero-pent-2-
enofuranosyl]cytosine Chimeric oligonucleotide Gapmers

| Entry | Sequence | | Target | Class |
|---|---|---|---|---|
| 114 | 5 C⁰T⁰A⁰G⁰A⁰TTCCACACTCT⁰C⁰G⁰T⁰C* 3 | (SEQ ID NO:33) | Mur. MDM2 | Gapmer |

C* = 1[2,3-deoxy-2-N-morpholino-β-D-glycero-pent-2-enofuranosyl]cytosine,
All P = S,
C⁰ = 2′-O-MOE ⁵MeC,
A⁰ = 2′-O-MOE A,
T⁰ = 2′-O-MOE ^{5Me}U,
G⁰ = 2′-O-2′-O-MOE G.

### EXAMPLE 51

### Preparation of CPG Resin Substituted with 9-(Aminoethoxy)phenoxazine Nucleotide (G-Clamp), G-Clamp Succinate 154

After drying at 50° C. in vacuo overnight, the G-clamp 2′-deoxynucleoside (152, 0.51 g, 0684 mmol) was dissolved in anhydrous DMC/Pyr (5:1) and 0.103 g (1.03 mmol) succinic anhydride were added to the solution. Subsequently 41.5 mg (0.34 mmol) DMAP in 1 mL DMF were added and the mixture was stirred overnight. After completion of the reaction (TLC) the solvent was evaporated in vacuo and the remaining yellow oil was dissolved in DCM, washed twice with 10% aq. NAHCO₃, 10% aq. citrate and brine. After drying over Na₂SO₄ the organic phase was evaporated in vacuo to yield a yellow solid (0.45 g, 75%). MS (HR-FAB) m/z 897.256 (M+Na)⁺.

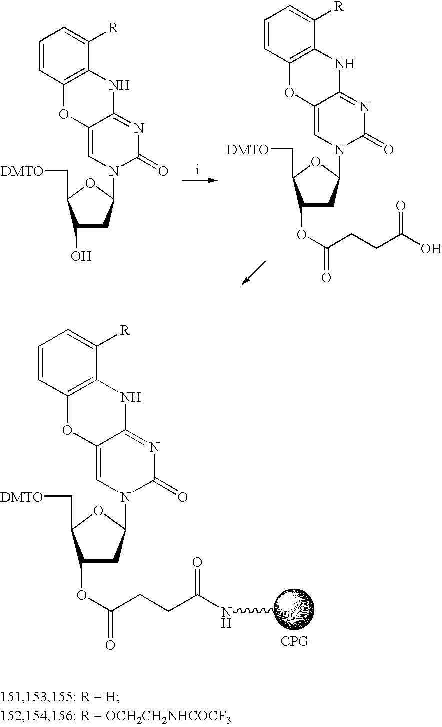

### EXAMPLE 52

### G-Clamp-Succinyl-LCAA-CPG 156

131 mg (0.15 mmol) G-clamp succinate were dissolved in DMF and 68 μL (0.4 mmol) DIEA were added. Subsequently a solution of 57 mg (0.15 mmol) HATU in DMF was added to the mixture under stirring. Stirring was continued for about 1 min in order to allow pre-activation before the mixture was added to 1 g of LCAA-CPG (initial loading: 115 μmol/g) and the suspension was shaken overnight. Subsequently the resin was washed 3 times each with DMF, DCM and CH₃CN and the unreacted amino groups of the resin were capped by shaking the resin with 0.24 mL (2 mmol) ethyl trifluoroacetate and 0.28 ml (2 mmol) TEA in 5 ml MeOH. Finally the resin was washed with MeOH, CH₃CN and DCM and dried in vacuo. The loading with G-clamp succinate was determined by DMT assay (final loading: 65 μmol/g).

### EXAMPLE 53

### 2′-deoxy Phenoxazine CPG

2′-deoxy phenoxazine CPG was synthesized following the procedures illustrated in example 52 above.

### EXAMPLE 54

### Oligonucleotide Synthesis

Solid phase syntheses of oligonucleotides containing G-clamp and phenoxazine units were carried out using standard phosphoramidite chemistry and an Applied Biosystems (Perkin Elmer Corp.) DNA/RNA synthesizer 380B. Cleavage and deprotection of the oligonucleotides was performed using a solution of 40% aq. MeNH₂ and 28-30% aq. NH₃ (1:1) at r.t. for 4 h. The oligonucleotides were purified by reversed phase HPLC using a 306 Piston Pump System, a 811C Dynamic Mixer, a 170 Diode Array Detector and a 215 Liquid Handler together with the Unipoint Software from Gilson (Middleton, Wis.). The HPLC conditions were as follows: Column: Waters Deltapak C₁₈ reversed phase (300×3.9 mm, 15 μ, 300 Å); Solvent A: 0.1 M NH₄OAc in H₂O; solvent B: 0.1 M NH₄OAc in CH₃CH/H₂O (80:20); Gradient: 0-40 min 0-50% B. After chromatographic purification the oligonucleotides were desalted by RP-HPLC, lyophilized, and stored at −20° C.

### EXAMPLE 55

### Guanidinylation of Solid Support

As outlined in Scheme 21, we have used two different strategies to introduce the guanidinium moiety. One strategy is the selective deprotection of the primary amino group followed by guanidinylation on the solid support (A). In the case of the 2′-O-(aminohexyl) function the allyloxycarbonyl (Alloc) protecting group was selectively removed by treating the support-bound oligonucleotides with 1.0 mL of 10 mg Pd₂[(Ph—CH═CH)₂CO]₃ and 26 mg P(Ph)₃ in a solution of 1.2 M nBuNH₂/HCOOH in THF at 50° C. for 1.5 h. After the removal of Alloc, the support-bound oligonucleotides were washed with DCM, acetone, sodium N,N-diethyldithio-carbamate (ddtc Na⁺), H₂O, acetone, DCM, diethyl ether and dried in vacuo. Prior to guanidinylation, the resin was suspended in a solution of 10% DIEA in DMF, shaken for 5 min, and washed with DMF followed by DCM. Subsequently, a 1.0 M solution of 1H-pyrazole-1-carboxamidine hydrochloride and DIEA in DMF was added to the support-bound oligonucleotides and the suspension was shaken at r.t. for 5 h. For final deprotection and cleavage of the oligonucleotides, the resin was treated with conc. aqueous ammonia at 55° C. for 1 h. After separation from the CPG support and evaporation of ammonia, the aqueous solution was filtered through a 0.45 μm Nylon-66 filter and stored frozen at −20° C. for further analysis.

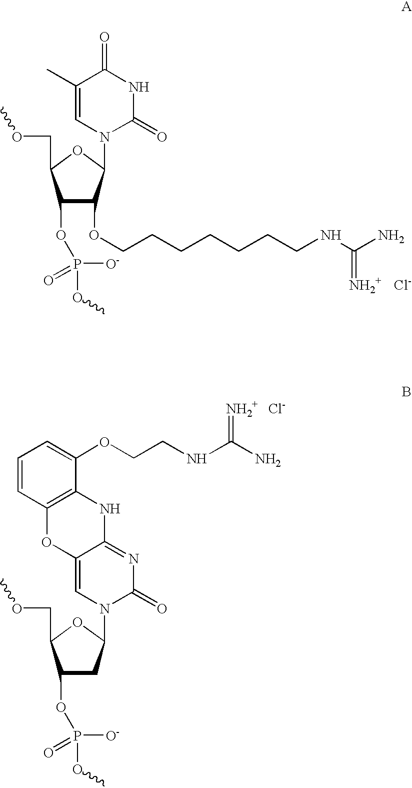

Modified nucleotides 2′-O-(guanidinylhexyl)-5-methyluridine (A); 9-guanidinylethoxy phenoxazine nucleotide (B)

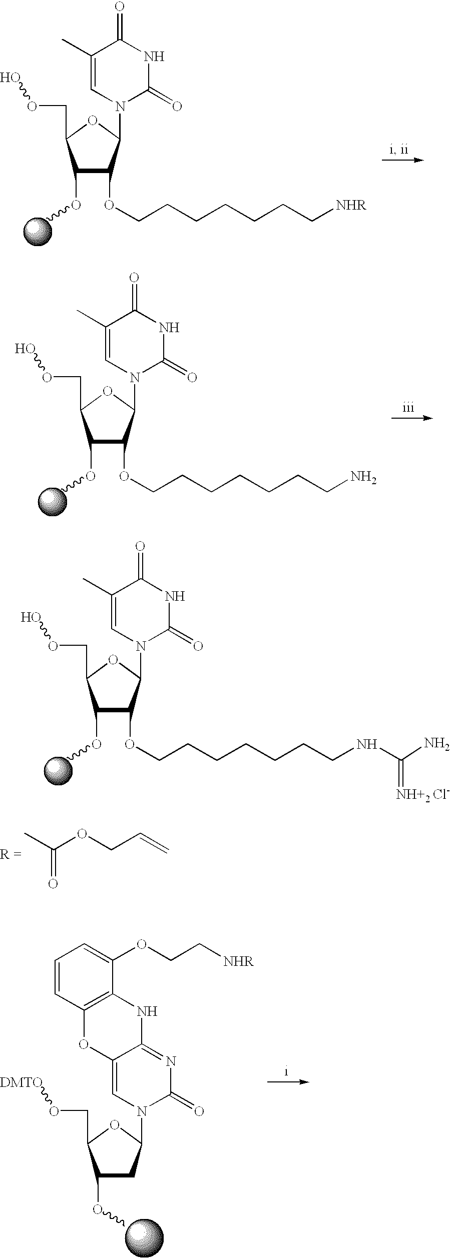

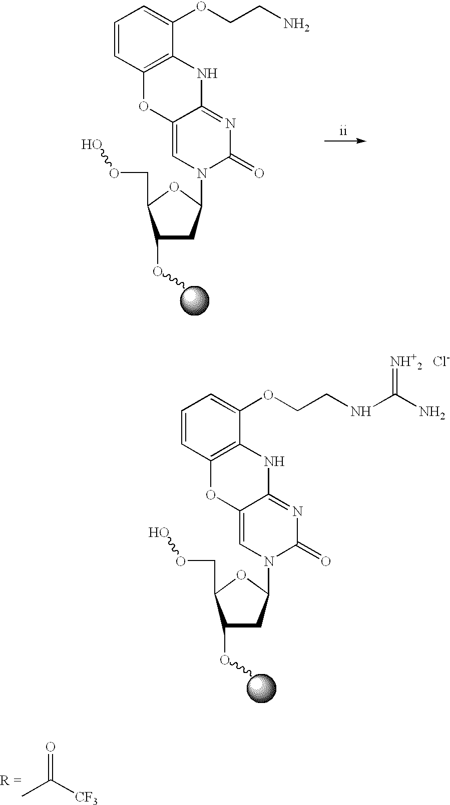

(A) Reaction conditions: (i) 1.0 mL of 10 mg Pd₂[(Ph—CH═CH)₂CO]₃, 26 mg P(Ph)₃ in 1.2 M nBuNH₂/HCOOH in THF, 50° C., 1.5 h; (ii) washing with DCM, acetone, sodium N,N-diethyldithiocarbamate (ddtc Na⁺), H₂O, acetone, DCM, diethyl ether, (iii) 1.0 M of 1H-pyrazole-1-carboxamidine hydrochloride and DIEA in DMF, r.t., 5 h (B) (i) 40% aq. CH₃—NH₂/conc. aq. NH₃ (1:1), 55° C., 1 h; (ii) 1.0 M 1H-pyrazole-1-carboxamidine hydrochloride in 1.0 M aq. Na₂CO₃, r.t., 3 h for ON-3, ON-4 and 55° C., 12 h for ON-5, ON-6, respectively.

### EXAMPLE 56

### Guanidinylation of Completely Deprotected Oligonucleotide in Solution

The base-labile trifluoroacetyl group (Tfa), which is compatible with the conditions of oligonucleotide synthesis and deprotection, was chosen for protection of the primary amino group of G-clamp. The oligonucleotide were deprotected and cleaved from the solid support prior to guanidinylation by using a 1:1 mixture of 40% aqueous CH₃—NH₂ and conc. aqueous ammonia (AMA), which prevents the formation of acyl- or acrylonitrile adducts with the highly nucleophilic primary amino group. To avoid transamination at cytosine during the deprotection step, N-acetyl- instead of N-benzoyl-protected C was used for oligonucleotide synthesis. After the oligomers were purified by RP-HPLC and analyzed by ES-MS, the primary amino group of G-clamp was guanidinylated by treating the oligonucleotides with 1-2 μmol of 2 mmol (297 mg) of 1H-pyrazole-1-carboxamidine hydrochloride in 2 mL of a 1.0 M aqueous Na₂CO₃ solution at r.t. for 3 h. Subsequently, the oligonucleotides were purified by gel chromatography (Sephadex G25) followed by RP-HPLC and analyzed by capillary gel electrophoresis (CGE) and electrospray mass spectrometry (ES-MS). The guanidynyl-modified oligonucleotides synthesized during this study are summarized in Table XVI.

Interestingly, in the case of self-complementary sequences, such as ON-5 (SEQ ID NO:38) or ON-6 (SEQ ID NO:39), the conditions described above yielded only a small fraction of guanidinyl G-clamp oligomer. Apparently, the double-stranded structure of these palindromic oligonucleotides with the primary amino group being involved in base pairing interaction with complementary guanine prohibited guanidinylation. In order to disrupt hydrogen bond interaction and to prevent duplex formation, the reaction was carried out at elevated temperature of 55° C. and extended reaction time of about 12 h. Using these conditions, complete guanidinylation of the amino groups of ON-5 (SEQ ID NO:38) and ON-6 (SEQ ID NO:39) was achieved without causing any detectable side reactions.

Guanidinylation of the primary amino groups slightly increased the hydrophobicity of the corresponding oligomers, which could be detected by RP-HPLC analysis as a minor change in the retention time. The Tₘ data of ON-3 in comparison to the unmodified G-clamp ON-2 (SEQ ID NO:35) show a decrease in hybridization affinity towards complementary RNA and DNA of 5.9 and 5.7° K., respectively (Table XVII). These findings, which seem to be contradictory to the formation of the additional hydrogen bonding between guanidinyl G-clamp and a complementary guanine, could be explained by another structural detail observed by crystallographic X-ray analysis of the duplex of self-complementary ON-5 (SEQ ID NO:38) [Wilds, C. J.: Maier, M. A.; Tereshko, V.; Manoharan, M.; Egli, M. in preparation]. The modified base pairs C* and G showed some buckling relative to the other base pairs in the duplex, which might be a consequence of altered steric requirements for accommodating the guanidinium-ethoxy moiety within the geometric boundaries of both the Watson-Crick and Hoogsteen-type hydrogen bonds. It can be assumed that the out-of-plane distortion is responsible for the loss of affinity observed for the guanidinyl-modified ON-3 (SEQ ID NO:36) compared to the parent G-clamp containing ON-2 (SEQ ID NO:35).

In summary, two methods for postsynthetic modification of oligonucleotides have been developed, which involve the conversion of primary amino functions into guanidinium groups by using 1H-pyrazole-1-carboxamidine hydrochloride. For reaction on the solid support, the amino groups were protected by Alloc, which can be selectively removed without cleaving the oligonucleotide from the support, and the guanidinylation was carried out in 10% DIEA in DMF. On the other hand, primary amino groups were protected with Tfa, which can be readily removed under the conditions of oligonucleotides bearing guanidinium moieties, facing either the minor or major groove, have been prepared and analyzed.

TABLE XV1

Oligonucleotide Sequence and Guanidinyl Modification.

| | | | | |
|---|---|---|---|---|
| OLIGO | Sequence 5′→3′ | Modification | MW_{calc} | MW_{found} |

| | | | | |
|---|---|---|---|---|
| ON-1 | TTT TU*T TTT T | all PO; U*: 2′-O- | 3281.6 | 3281.7 |
| | (SEQ ID NO:34) | hexylguanidinyl-U^{5me} | | |
| ON-2 | TCT CC*C TCT C | all PO; C* = 2′-deoxy- | 3039.1 | 3039.4 |
| | (SEQ ID NO:35) | G-clamp | | |
| ON-3 | TCT CC*C TCT C | all PO; C* = 2′-deoxy- | 3081.1 | 3080.8 |
| | (SEQ ID NO:36) | guanidinyl G-clamp | | |
| ON-4 | CTC GTA CCC* TCC | all PO; C* = 2′-deoxy- | 5553.7 | 5552.1 |
| | CGG TCC (SEQ ID | guanidinyl G-clamp | | |
| | NO:37) | | | |
| ON-5 | GC*G TAU_{M} ACGC | all PO; U_{M} = 2′-MOE-U^{5me}; | 3293.3 | 3292.8 |
| | (SEQ ID NO:38) | C* = 2′-deoxy- | | |
| | | guanidino G-clamp | | |
| ON-6 | GCG TAU_{M} AC*GC | all PO; U_{M} = 2′-MOE-U^{5me}; | 3293.3 | 3293.0 |
| | (SEQ ID NO:39) | C* = 2′-deoxy- | | |
| | | guanidino G-clamp | | |

### EXAMPLE 58

TABLE XVII
Tₘ Data of ON-3 (SEQ ID NO:36) in comparison to the parent G-clamp-
modified ON-2 (SEQ ID NO:35).

| ON | Modification | Target Strand^{a} | Tₘ | ΔTₘ/mod^{b} |
|---|---|---|---|---|
| ON-2 | G-Clamp | RNA | 70.8 | 18.4 |
| ON-3 | Guanidinyl G-clamp | RNA | 64.9 | 12.5 |
| ON-2 | G-Clamp | DNA | 59.2 | 22.1 |
| ON-3 | Guanidinyl G-clamp | DNA | 53.5 | 16.4 |

^{a}Sequence: 5′-AAAAA GAG AGG GAG A (SEQ ID NO:40); ^{b}vs. parent DNA.

### EXAMPLE 59

### Guanidinyl G-clamp Modification

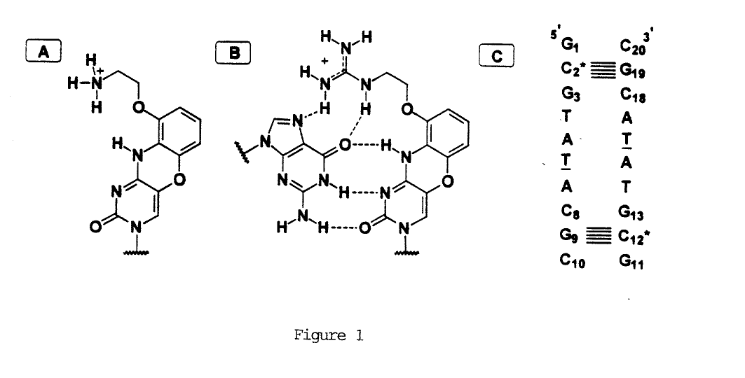

The guanidinyl G-clamp modifications was designed to allow for additional hydrogen bonds to the O6 and N7 Hoogsteen binding sites of guanosine (FIG. 1B). Binding studies of DNA oligomers containing a single unit to a RNA target revealed an increase in the melting temperature of 16° C. relative to the wildtype DNA, slightly lower than the ΔTₘ observed for the original G-clamp modification. To investigate the structural properties of this modification we determined the X-ray crystal structure of a modified decamer duplex with the sequence GC*GTAT_{MOE}ACGC (SEQ ID NO:41), where C* is the guanidino G-clamp and a 2′-O-methoxyethyl thymine is T_{MOE} (FIG. 1C). Altmann, K. -H.; Dean, N. M.; Fabbro, D.; Freier, S. M.; Geiger, T.; Häner, R.; Husken, D.; Martin, P.; Monia, B. P.; Müller, M.; Natt, F.; Nicklin, P.; Phillips, J.; Pieles, U.; Sasmor, H.; Moser H. E. Chimia 1996, 50, 168-176; Teplova, M.; Minasov, G.; Tereshko, V.; Inamati, G. B.; Cook, P. D.; Manoharan, M.; Egli. M. Nature Struct. Biol. 1999, 6, 535-539. The synthesis and purification of the oligonucleotides was carried out according to standard procedures. Crystals of this decamer duplex were grown by the hanging drop vapor diffusion method using commercially available screen (Hampton Research, Laguna Niguel, Calif.) [Hanging drop vapor diffusion: a 2 μL droplet (1.2 mM DNA, 5% MPD, 20 mM Na cacodylate pH 6.0, 6 mM spermine·4 HCl, 40 mM NaCl, 6 mM KCl, 10 mM MgCl₂ was equilibrated against a reservoir of 1 mL 35% v/v MPD. Space group P2ₗ2ₗ2ₗ; cell dimensions a=24.52 Å, b=43.02 Å, c=46.68 Å]. Data collection was performed synchrotron source [A crystal (0.7×0.2×0.2 mm) was picked up from a droplet with a nylon loop and transferred into a cold N₂ stream (120 K). High- and low-resolution data sets were collected on the 5-ID beam line (λ=0.978 Å) of the DND-CAT at the Advanced Photon Source, Argonne, Ill., using a MARCCD detector. Data were integrated and merged with DENZO/SCALEPACK¹⁰. The overall R_{merge} for all reflections between 20 and 1 Å was 4.7% (Otwinowski, Z.; Minor W. Methods Enzymol. 1997, 276, 307-326) and data collection and refinement statistics are listed in Table XVIII. The structure was solved by molecular replacement using the DNA decamer as the initial model and refined with the programs CNS¹² and SHELX-97¹³. After monitoring the R_{free} using 10% of the reflections and reaching 22%, all reflections were included in the final rounds of isotropic refinement; Brünger, A. T. Crystallography & NMR System (CNS), Version 0.9, Yale University, New Haven, Conn., 1998 [Sheldrick, G. M.; Schneider, T. R. Methods Enzymol. 1997, 277, 319-343; Egli, M.; Tereshko, V.; Teplova, M.; Minasov, G.; Joachimiak, A.; Sanishvili, R.; Weeks, C. M.; Miller, R.; Maier, M. A.; An, H. Y.; Cook, P. D.; Manoharan, M. Biopolymers: Nucleic Acids Sciences 1998, 48, 234-252; Clarke, N. D.; Beamer, L. J.; Goldberg, H. R.; Berkower, C.; Pabo, C. O. Science 1991, 254, 267-270; Rich, A. In The Chemical Bond: Structure and Dynamics; Zewail, A. Ed.; Academic Press, New York, 1992; pp 31-86; Pabo, C. O.; Sauer, R. T. Annu. Rev. Biochem. 1992, 61, 1053-1059; Lin, K. -Y.; Jones, R. J.; Matteucci, M. D. J. Am. Chem. Soc. 1995, 117, 3873-3874].

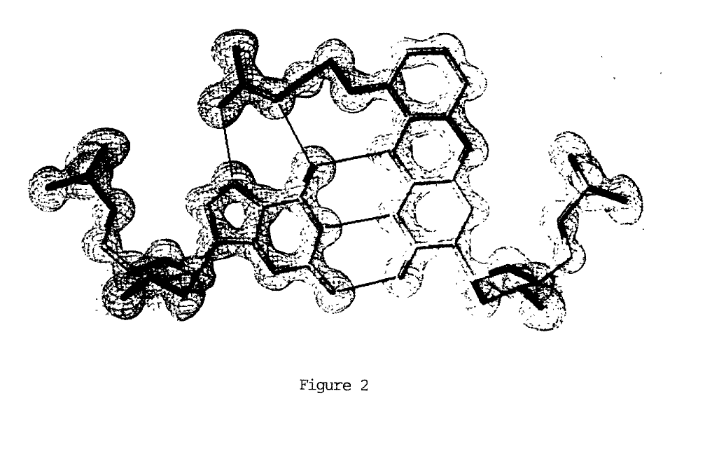

The overall structure of this duplex is A-form as a result of 2′-O-methoxyethyl thymine units at positions 6 and 16 in the duplex. An A-form environment is desirable to study the structure of nucleic acid modifications for antisense purposes. As illustrated in the case of base pair C12*-G9 (FIG. 2), electron density around the heterocycle clearly shows the two Hoogsteen-type hydrogen bonds formed between the amino and imino nitrogen of the tethered guanidinium and O6 and N7 of guanosine, respectively. The hydrogen bond lengths are 2.88 Å and 2.86 Å and the lengths of the corresponding hydrogen bonds in base pair C2*-G19 and 2.92 Å and 2.87 Å, respectively. The quality of the electron density around individual atoms of the phenoxazine ring and tethered group demonstrate that this modification is well ordered and does not assume random conformations. There is some buckling of modified base pairs relative to the other base pairs in the duplex. This out-of-plane distortion of the base pair between the G-clamp and G may be a consequence of the requirement to optimize the geometry of both the Watson-Crick and Hoogsteen-type hydrogen bonds within the geometric boundaries provided by a guanidinium-ethoxy moiety. In addition, the observed arrangements help avoid a steric contact between O6 of G and the ethoxy-linker oxygen of the G-clamp (FIGS. 1 and 2).

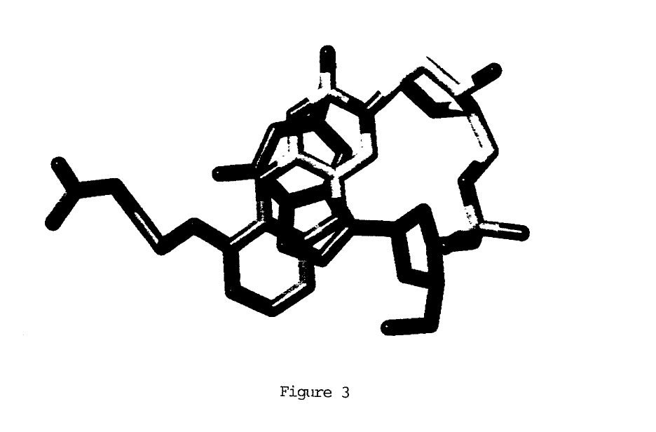

Presence of the G-clamp results in a considerable improvement of intra-strand stacking at the GpC* step compared with stacking between cytosine and the 5′-adjacent base (G1 and G11, respectively). The overlap between G1 and C2* is depicted in FIG. 3. While the “cytosine core” displays relatively little stacking to the guanosine base, the remainder of the phenoxazine ring system virtually covers the entire guanosine base. However, while stacking between G-clamp and the base to the 5′-side is improved, stacking to the 3′-adjacent base is not affected by incorporation of the modified base.

Placement of the positively charged guanidinium moiety in the center of the major groove, a site of strong negative potential, likely results in a significant electrostatic contribution to stability. Moreover, the guanidinium group and phosphates from opposite strands are relatively closely spaced. The average distance between the imino nitrogens of C* and O2P oxygens of phosphates is 5.8 Å. Although too long for direct salt bridges, water molecules mediate contacts between a water bound between guanidinium imino nitrogens and O2P oxygens of residues C8 and G9.

Interactions between positively charged amines and the Hoogsteen binding site of guanosine are well known. For example, X-ray crystallographic studies of the λ repressor bound to duplex DNA revealed specific contacts between a lysine and the O6 position of G [Clarke, N. D.; Beamer, L. J.; Goldberg, H. R.; Berkower, C.; Pabo, C. O. Science 1991, 254, 267-270; Rich, A. In The Chemical Bond: Structure and Dynamics; Zewail, A. Ed.; Academic Press, New York, 1992, 31-86; Pabo, C. O.; Sauer, R. T. Annu. Rev. Biochem. 1992, 61, 1053-1059]. The present structure of the guanidyl G-clamp is similar to the bidentate hydrogen bonding of the arginine fork with the N7 and O6 positions of guanine in protein-nucleic acids interactions [Clarke, N. D.; Beamer, L. J.; Goldberg, H. R.; Berkower, C.; Pabo, C. O. Science 1991, 254, 267-270; Rich, A. In The Chemical Bond: Structure and Dynamics; Zewail, A. Ed.; Academic Press, New York, 1992, 31-86; Pabo, C. O.; Sauer, R. T. Annu. Rev. Biochem. 1992, 61, 1053-1059]. The observed structure reveals some buckling of the C*-G base pair, presumably due to sterics as a consequence of the extended guanidinylethoxy spacer arm. A comparison of the Tₘ data of the G-clamp and guanidino G-clamp revealed that guanidinylation appears to have only a slight effect on overall stability.

Two crucial stabilizing factors of this modification are an increase in the number of hydrogen bonds and improved stacking interactions. Additional contributions to stability are favorable electrostatic interactions and well-ordered water networks. It is difficult to discern if one of these contributions plays a more important role than the others. Binding studies of oligomers with the phenoxazine moiety alone showed moderate increases in Tₘ of 2-7° C [Lin, K. -Y.; Jones, R. J.; Matteucci, M. D. J. Am. Chem. Soc. 1995, 117, 3873-3874]. Stability was increased most when several phenoxazine groups were clustered together on the same strand, allowing for tricyclic-tricyclic stacking interactions. In the case of an acyclic G-clamp modification, no enhancement in binding was observed. Only when both the phenoxazine and tethered amino group were present was a drastic improvement in binding observed [Lin, K. -Y.; Matteucci, M. D. J. Am. Chem. Soc. 1998, 120, 8531-8532; Flanagan, W. M.; Wolf, J. J.; Olson, P.; Grant, D.; Lin, K.; Wagner, R. W.; Matteucci, M. D. Ti Proc. Natl. Acad. Sci. U.S.A. 1999, 96, 3513-3518]. Clearly, hydrogen bonds from the guanidinium group maintain the guanidino G-clamp modification in a position that allows stacking interactions and formation of stable water networks. This is the first report of a single base pair within a nucleic acid duplex combining Watson-Crick and Hoogsteen binding to a total number of five hydrogen bonds.

### EXAMPLE 60

### Reflection Data and Refinement Statistics

TABLE XVIII

| Resolution | | Mean | | |
|---|---|---|---|---|
| (Å) | N (unique) | [I/σ(I)] | % complete | R-factor^{a} |
| 10.00-3.00 | 1073 | 26.90 | 98.8 | 0.175 |
| 3.00-2.50 | 768 | 31.51 | 99.9 | 0.182 |
| 2.50-2.00 | 1722 | 34.38 | 100.0 | 0.180 |
| 2.00-1.80 | 1288 | 36.70 | 100.0 | 0.154 |
| 1.80-1.60 | 2005 | 30.33 | 99.9 | 0.153 |
| 1.60-1.40 | 3314 | 27.90 | 100.0 | 0.166 |
| 1.40-1.20 | 5804 | 24.68 | 100.0 | 0.179 |
| 1.20-1.10 | 4680 | 20.08 | 100.0 | 0.187 |
| 1.10-1.00 | 6666 | 14.35 | 99.6 | 0.200 |
| All data | 27320 | 23.63 | 99.6 | 0.175 |

^{a}R-factor = Σₕₖₗ | F(hkl)ₒ − F(hkl)_{c} |/Σₕₖₗ F(hkl)ₒ; no σ cutoff was used.

### EXAMPLE 61

Synthesis of G-clamp Modified Oligonucleotides
targeting c-raf Message

| | | |
|---|---|---|
| Sequence (5′-3′) | Backbone | Modification |

| | | |
|---|---|---|
| ATG-CAT-TCT-GCC-CCC-AAG-GA | P = S | (SEQ ID NO:42) |
| ATG-C*AT-TCT-GCC-CCC-AAG-GA | P = S | (SEQ ID NO:43) |
| ATG-CAT-TC*T-GCC-CCC-AAG-GA | P = S | (SEQ ID NO:44) |
| ATG-CAT-TCT-GC*C-CCC-AAG-GA | P = S | (SEQ ID NO:45) |
| ATG-CAT-TCT-GCC*-CCC-AAG-GA | P = S | (SEQ ID NO:46) |
| ATG-CAT-TCT-GCC-C*CC-AAG-GA | P = S | (SEQ ID NO:47) |
| ATG-CAT-TCT-GCC-CC*C-AAG-GA | P = S | (SEQ ID NO:48) |
| ATG-CAT-TCT-GCC-CCC*-AAG-GA | P = S | (SEQ ID NO:49) |

C* = G-clamp modification.

### EXAMPLE 62

### In vivo Stability of Modified MDM-2 Oligonucleotides

TABLE XIX

2′-deoxy Oligonucleotides for in vivo
Stability Evaluation

| | | |
|---|---|---|
| Sequence (5′-3′) | Target | Backbone |

| | | |
|---|---|---|
| CTA GAT TCC ACA CTC TCG TC | MDM-2 | P = S |
| (SEQ ID NO:50) | | |
| C*TA GAT TCC ACA CTC TCG TC | MDM-2 | P = S |
| (SEQ ID NO:51) | | |
| CTA GAT TCC ACA CTC TCG TC* | MDM-2 | P = S |
| (SEQ ID NO:52) | | |
| C*TA GAT TCC ACA CTC TCG TC* | MDM-2 | P = S |
| (SEQ ID NO:53) | | |

C* = G-clamp modification.

The in vivo stability of selected modified oligonucleotides synthesized is determined in BALB/c mice. Following a single i.v. administration of 5 mg/kg of oligonucleotide, blood samples are drawn at various time intervals and analyzed by CGE.

For each oligonucleotide tested, 9 male BALB/c mice (Charles River, Wilmington, Mass.) weighing about 25 g are used. Following a one week acclimatization the mice received a single tail-vein injection of oligonucleotide (5 mg/kg) administered in phosphate buffered saline (PBS), pH 7.0. One retro-orbital bleed (either at 0.25, 0.5, 2 or 4 h post-dose) and a terminal bleed (either 1, 3, 8, or 24 h post-dose) are collected from each group. The terminal bleed (approximately 0.6-0.8 mL) is collected by cardiac puncture following ketamine/xylazine anasthesia. The blood is transferred to an EDTA-coated collection tube and centrifuged to obtain plasma. At termination, the liver and kidneys are collected from each mouse. Plasma and tissue homogenates are used for analysis to determine intact oligonucleotide content by CGE. All samples are immediately frozen on dry ice after collection and stored at −80° C. until analysis.

The CGE analysis indicated the relative nuclease resistance of G-clamp modification containing oligomers compared to the parent MDM-2 (uniformly 2′-deoxy-phosphorothioate oligonucleotide targeted to mouse MDM-2). Because of the nuclease resistance of the G-clamp modification, the modified oligonucleotides are found to be more stable in plasma, while ISIS 11061 (SEQ ID NO:42) was not. Similar observations are noted in kidney and liver tissue. This implies that G-clamp modifications offer excellent nuclease resistance in plasma, kidney and liver against exonucleases and endonucleases. Thus oligonucleotides with longer duration of action can be designed by incorporating both the G-clamp modification and other analogous motifs into their structure. A plot of the percentage of full length oligonucleotide remaining intact in plasma one hour following administration of an i.v. bolus of 5 mg/kg oligonucleotide is determined to evaluate the stability in plasma.

A plot of the percentage of full length oligonucleotide remaining intact in tissue 24 hours following administration of an i.v. bolus of 5 mg/kg oligonucleotide is determined. CGE traces of test oligonucleotides and a standard phosphorothioate oligonucleotide in both mouse liver samples and mouse kidney samples after 24 hours are evaluated. There is a greater amount of intact oligonucleotide for the oligonucleotides of the invention as compared to the standard of the parent unmodified. The maximum stability is seen when both 5′ and 3′ ends are capped with C*.

### EXAMPLE 63

Control of c-raf Message in bEND Cells using
G-clamp Modified Oligonucleotides

| | | | |
|---|---|---|---|
| ISIS # | Sequence (5′-3′) | Backbone | Sequence ID NO: |

| | | | |
|---|---|---|---|
| 11061 | ATG-CAT-TCT-GCC-CCC-AAG-GA | P = S | (SEQ ID NO:42) |
| ----- | ATG-C*AT-TCT-GCC-CCC-AAG-GA | P = S | (SEQ ID NO:43) |
| ----- | ATG-CAT-TC*T-GCC-CCC-AAG-GA | P = S | (SEQ ID NO:44) |
| ----- | ATG-CAT-TCT-GC*C-CCC-AAG-GA | P = S | (SEQ ID NO:45) |
| ----- | ATG-CAT-TCT-GCC*-CCC-AAG-GA | P = S | (SEQ ID NO:46) |
| ----- | ATG-CAT-TCT-GCC-C*CC-AAG-GA | P = S | (SEQ ID NO:47) |
| ----- | ATG-CAT-TCT-GCC-CC*C-AAG-GA | P = S | (SEQ ID NO:48) |
| ----- | ATG-CAT-TCT-GCC-CCC*-AAG-GA | P = S | (SEQ ID NO:49) |

C* = G-clamp modification

In order to assess the activity of some of the oligonucleotides, an in vitro cell culture assay is used that measures the cellular levels of c-raf expression in bEND cells.

Cells and Reagents

The bEnd.3 cell line, a brain endothelioma, is obtained from Dr. Werner Risau (Max-Planck Institute). Opti-MEM, trypsin-EDTA and DMEM with high glucose are purchased from Gibco-BRL (Grand Island, N.Y.). Dulbecco=s PBS is purchased from Irvine Scientific (Irvine, Calif.). Sterile, 12 well tissue culture plates and Facsflow solution are purchased from Polysciences (Warrington, Pa.). NAP-5 columns are purchased from Pharmacia (Uppsala, Sweden).

Oligonucleotide Treatment

Cells are grown to approximately 75% confluency in 12 well plates with DMEM containing 4.5 g/L glucose and 10% FBS. Cells are washed 3 times with Opti-MEM pre-warmed to 37° C. Oligonucleotide is premixed with a cationic lipid (Lipofectin reagent, (GIBCO/BRL) and, serially diluted to desired concentrations and transferred on to washed cells for a 4 hour incubation at 37° C. Media is then removed and replaced with normal growth media for 24 hours for northern blot analysis of mRNA.

Northern Blot Analysis

For determination of mRNA levels by Northern blot analysis, total RNA is prepared from cells by the guanidinium isothiocyanate procedure [Monia et. al., Proc. Natl. Acad. Sci. U.S.A. 1996, 93, 15481-15484] 24 h after initiation of oligonucleotide treatment. Total RNA is isolated by centrifugation of the cell lysates over a CsCl cushion. Northern blot analysis, RNA quantitation and normalization to G3PDH mRNA levels are done according to the reported procedure [Dean and McKay, Proc. Natl. Acad. Sci. U.S.A., 1994, 91, 11762-11766].

In bEND cells the G-clamp oligonucleotides showed reduction of c-raf message activity as a function of concentration. The fact that these modified oligonucleotides retained activity promises reduced frequency of dosing with these oligonucleotides which also show increased in vivo nuclease resistance. All G-clamp modified oligonucleotides retained the activity of the parent 11061 oligonucleotide (SEQ ID NO:42) and improved the activity even further.

### EXAMPLE 64

### Compound 201 (R′=CN, n=1, Scheme 22a, Table XX)

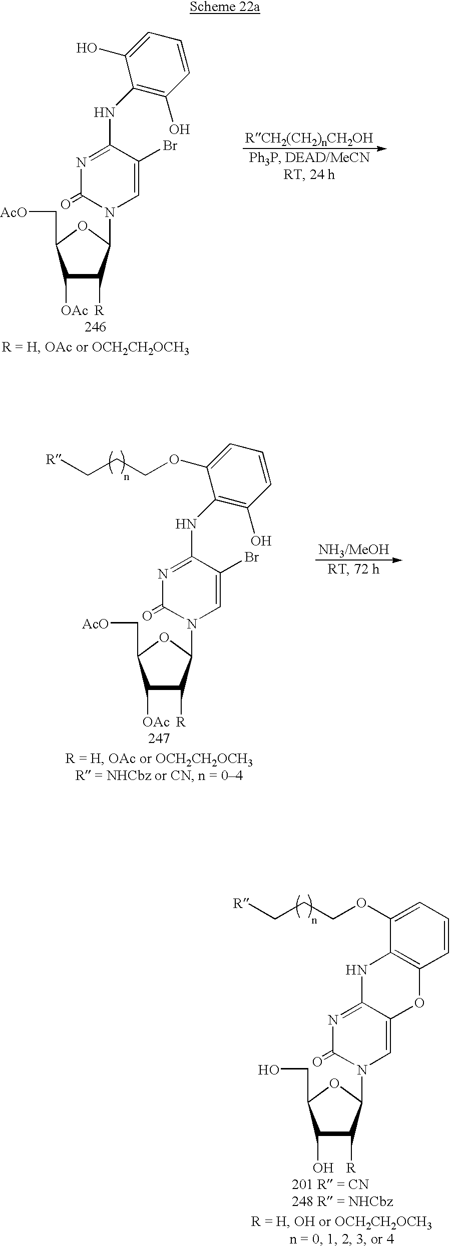 TABLE XX

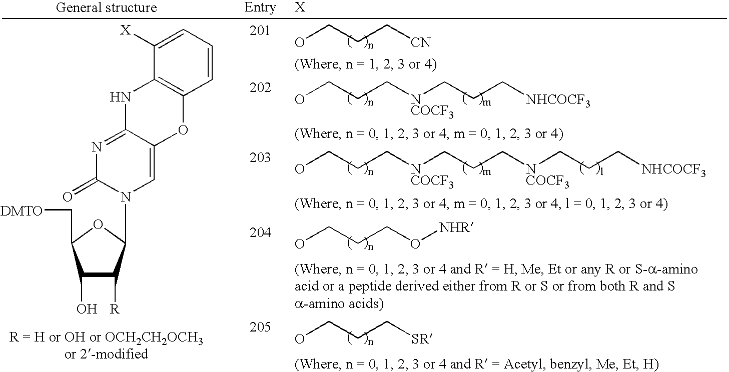

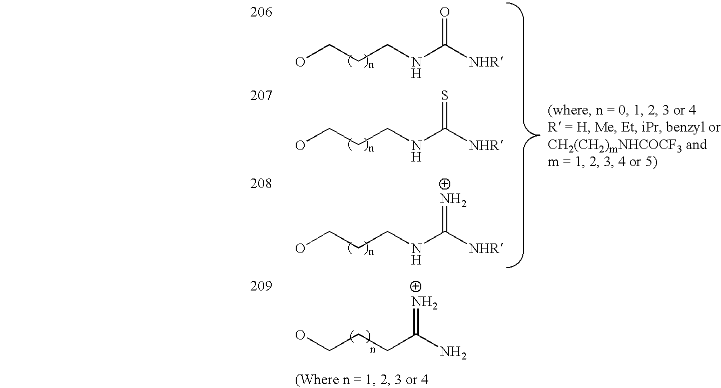

### EXAMPLE 65

### Compound 248 (R′=NHCbz, n=0, Scheme 22)

Compound 248 is prepared from compound 246 (1 mmol) and benzyl N-(2-hydroxyethyl)carbamate (1 mmol) according to the literature procedure [Lin and Matteucci, J. Am. Chem. Soc. 1998, 120, 8531-8532].

### EXAMPLE66

### Compound 249 (n=0, Scheme 22b)

Compound 248 (1 mmol) upon treatment with DMT-Cl (1 molar eq.) in pyridine yields the corresponding 5′-O-DMT derivative. The DMT derivative is stirred with ethyl trifluoroacetate in presence of TEA to obtain N-trifluoroacetyl-5′-O-DMT derivative of compound 248. Free 3′-hydroxy functional group of the product obtained is reacted with acetic anhydride in anhydrous pyridine to obtain the completely protected nucleoside 249.

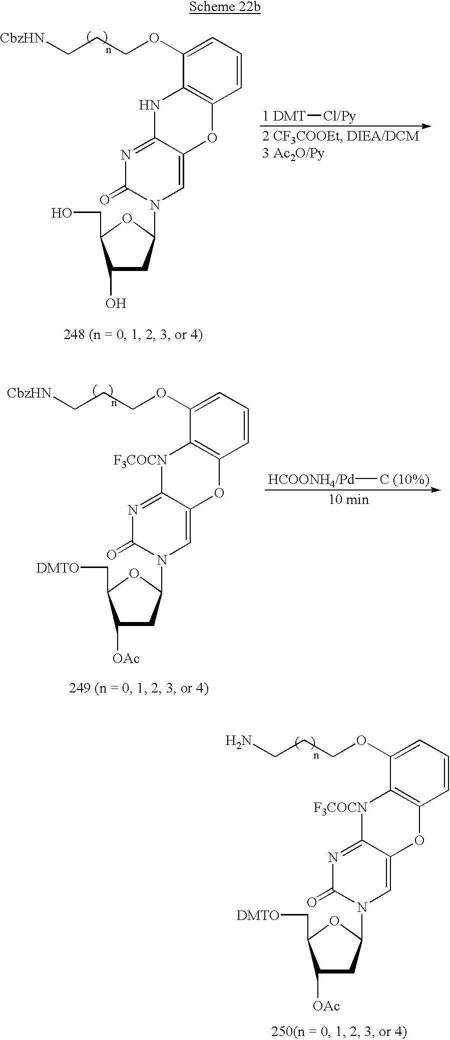

### EXAMPLE 67

### Compound 250 (n=0, Scheme 22b)

A suspension of compound 249 (1 mmol) and ammonium formate (5 mmol) in ethyl acetate is deoxygenated under argon and 10% palladium on charcoal (10 mol %) is added into the suspension under argon. The reaction mixture is stirred for 10 min at ambient temperature to obtain compound 250.

### EXAMPLE 68

### Compound 206 (n=0, R=Me, Scheme 22c, Table XX)

Compound 250 (1 mmol) in anhydrous THF is stirred with 1,1′-carbonyl-diimidazole (CDI, 1 mmol) under argon at ambient temperature for 2 h. After 2 h, the reaction mixture is cooled on an ice bath and anhydrous methylamine gas is bubbled

through the reaction mixture for 10 min. The resulting mixture is stirred for 30 min to obtain compound 206.

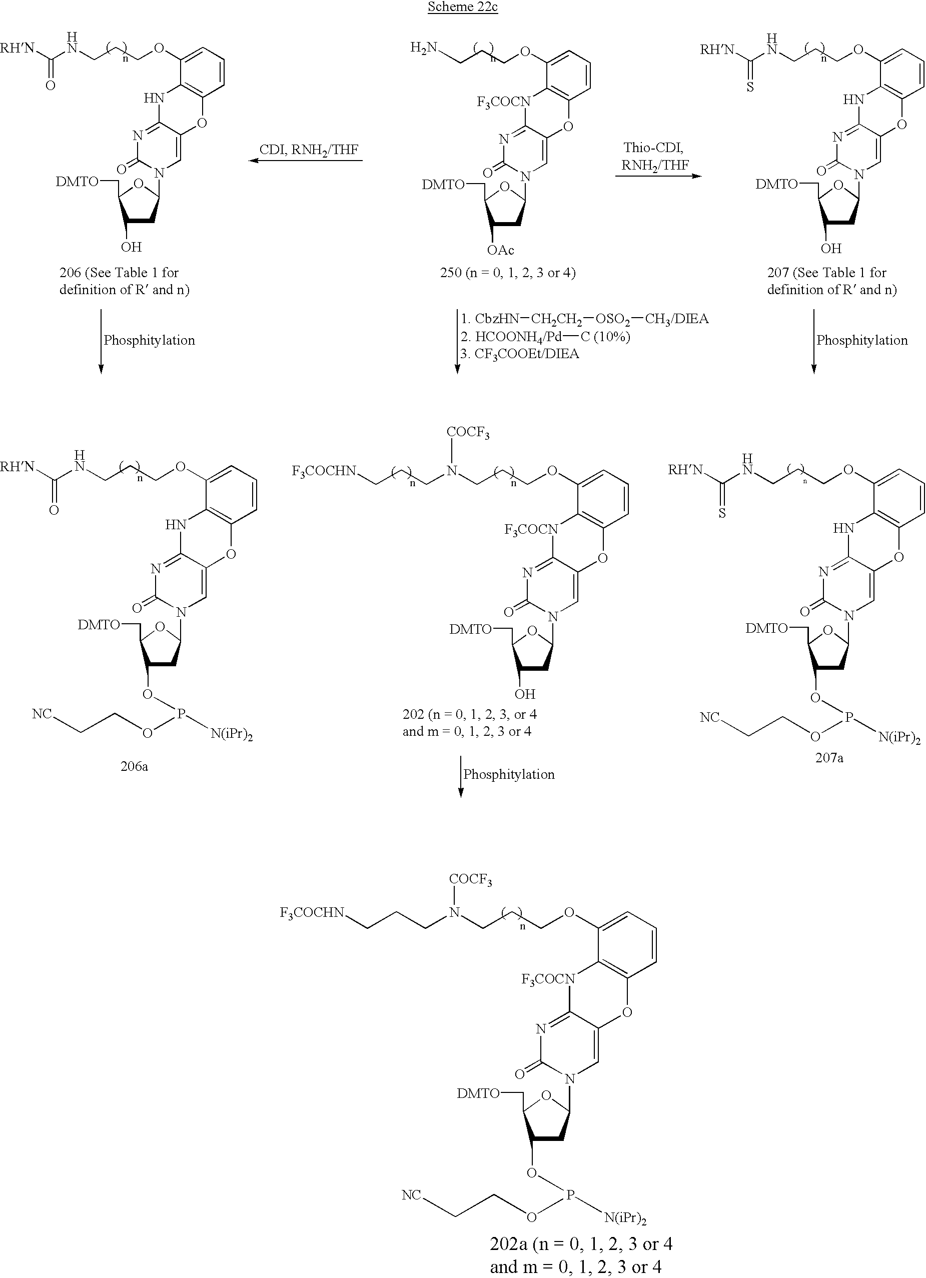

### EXAMPLE 69

### Compound 206a (n=0, R=Me, Scheme 22c)

Phosphitylation of the 3′-hydroxy group of compound 206 as described in Example 2 for the synthesis of compound 3 yields compound 206a.

### EXAMPLE 70

### Compound 207 (n=0, R=Me, Scheme 22c, Table XX)

Compound 207 is obtained from compound 250, 1,1′-thiocarbonyldiimidazole and methylamine under similar reaction conditions as described for the synthesis of compound 206 in Example 68.

### EXAMPLE 71

### Compound 207a (n=0, R=Me, Scheme 22c)

Phosphitylation of 3′-hydroxy group of compound 207 as described in Example 2 for the synthesis of compound 3 yields compound 207a.

### EXAMPLE 72

### Compound 202 (n=0, m=0, Scheme 22c, Table XX)

Compound 250 (1 mmol) is stirred with N-benzyloxycarbonyl-2-amino-ethanol-O-methane sulfonate (1 mmol) in presence DIEA in anhydrous DCM overnight. The secondary amine thus obtained is subjected to transfer hydrogenation as described in Example 59 to remove the benzyloxycarbonyl protection. The unprotected amine is then stirred with ethyl trifluoroacetate in presence of DIEA in DCM to obtain the desired compound 202.

### EXAMPLE 73

### Compound 202a (n=0, m=0, Scheme 22c)

Phosphitylation of compound 202 as described in Example 2 for the synthesis of compound 3 yields compound 202a.

### EXAMPLE 74

### Compound 208a (n=0, Scheme 22d, Table XX)

Compound 250 (1 mmol) and TEA (1 mmol) are added into a solution of compound A (1 mmol, Scheme 1d) and the resulting mixture is stirred at ambient temperature to obtain compound 208a.

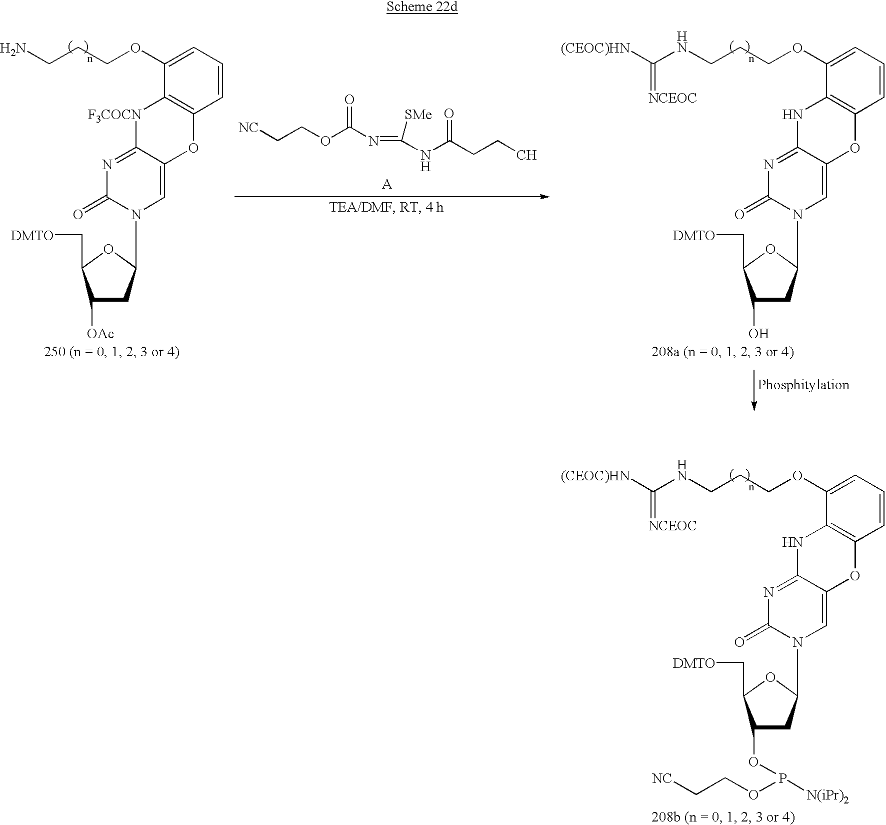

### EXAMPLE 75

### Compound 208b (n=0, Scheme 22d)

Phosphitylation of compound 208a as described in Example 2 yields compound 208b.

### EXAMPLE 76

### Compound 201a (n=1, Scheme 22e)

Reaction of compound 201 with DMTCl in pyridine yields compound 201a.

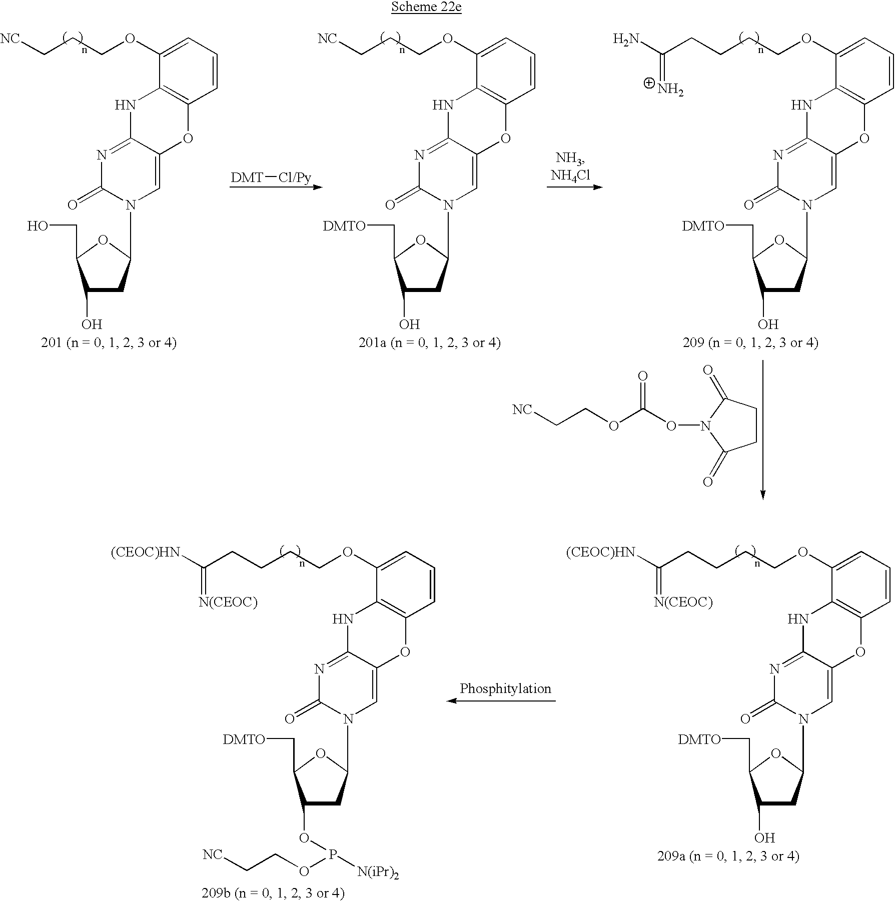

### EXAMPLE 77

### Compound 209 (n=1, Scheme 22e, Table XX)

Compound 201a is treated with ammonia and ammonium chloride in THF at elevated temperature under pressure to obtain compound 209 [Granik, Russ. Chem. Rev., 1983, 52, 377-393].

### EXAMPLE 78

### Compound 209a (n=1, Scheme 22e)

2-Cyanoethoxycarbonyloxysuccinimide (2 mmol) and DIEA are added into a solution of compound 209 (1 mmol) in DCM and the resulting mixture is stirred at ambient temperature to obtain compound 209a.

### EXAMPLE 79

### Compound 209b (n=0, Scheme 22e)

Phosphitylation of compound 209a as described in Example 2 for the synthesis of compound 3 yields compound 209b.

### EXAMPLE 80

### Compound 252 (Scheme 23a)

Phenoxazine nucleoside 252 with desired tether X is synthesized in five steps from 5-bromo-3′-O-TBDMS-5′-O-DMT-dU (251) according to the literature procedure by [Lin and Matteucci J. A. Chem. Soc., 1998, 120, 8531-8532].

### EXAMPLE 81

### Compound 253 (Scheme 23a)

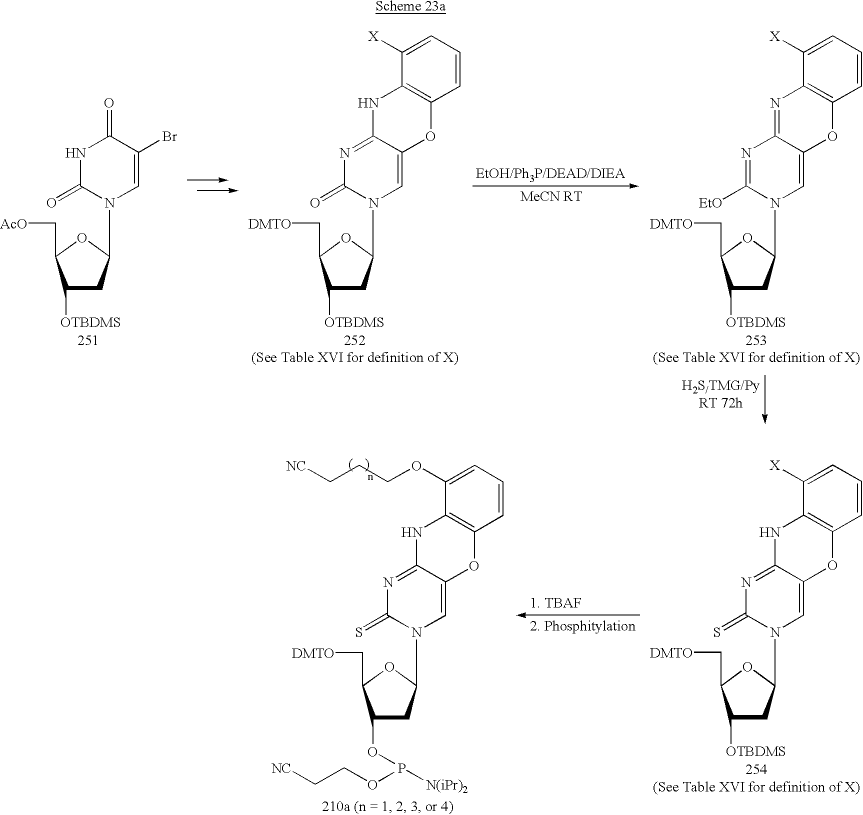

Reaction of compound 252 (1 mmol) with ethanol (1 mmol) under Mitsunobu alkylation condition (Ph₃P and DEAD 1 mmol each) in presence of DIEA in acetonitrile yields compound 253.

### EXAMPLE 82

### Compound 254 (Scheme 23a)

Compound 253 (1 mmol) after thorough drying over P₂O₅ under vacuum is taken in a reaction vessel under argon. TMG (10 mmol) in anhydrous pyridine, placed on a freezing bath, is saturated with anhydrous H₂S for 45 min. After 45 min, the resulting solution is transferred into the precooled vessel containing compound 253 under argon and is sealed. The sealed vessel is then brought to ambient temperature and is stored at ambient temperature for 3 days. Bubbles off the H₂S into a chlorox bath and removes pyridine from the reaction mixture under vacuum. The residue after standard work up and purification yields compound 254.

### EXAMPLE 83

### Compound 210a (n=1, Scheme 23a, Table XXI)

Compound 254 (X═O—(CH₂)₃—CN) is treated with TBAF in THF to remove the 3′-O-TBDMS group. The resulting 3′-OH group is subjected to phosphitylation under the conditions described in Example 2 to obtain compound 210a.

TABLE XXI

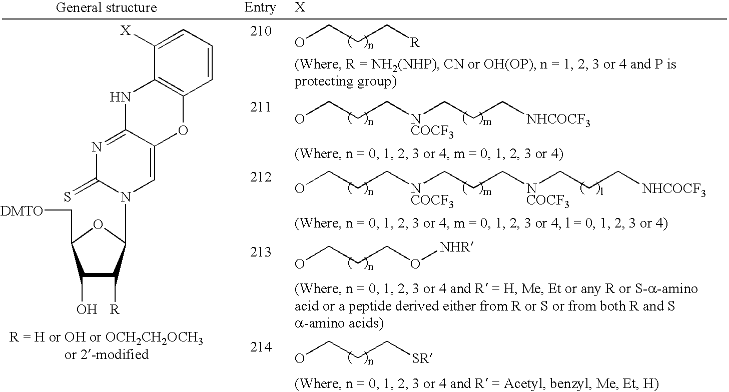

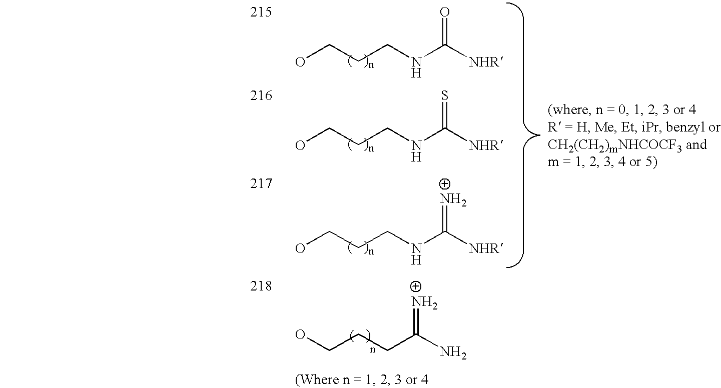

### EXAMPLE 84

### Compound 210b (n=0, Scheme 23b, Table XXI)

Compound 254 (1 mmol, n=0, Scheme 23b) is stirred with TBAF in THF to remove the 3′-O-protection. The resulting product is subjected to transfer hydrogenation using ammonium formate and Pd-C (10%) in ethyl acetate (See Example 67 for details) to remove the benzyloxycarbonyl protection from the side chain moiety. The free amine thus formed and the ring nitrogen are then protected as trifluoroacetamide by stirring the compound (1 mmol) with ethyl trifluoroacetate (10 mmol) in pyridine at ambient temperature. Finally the trifluoroacetamide derivative obtained is phosphitylated as described in Example 2 for the synthesis of compound 3 to obtain the desired phosphoramidite 210b.

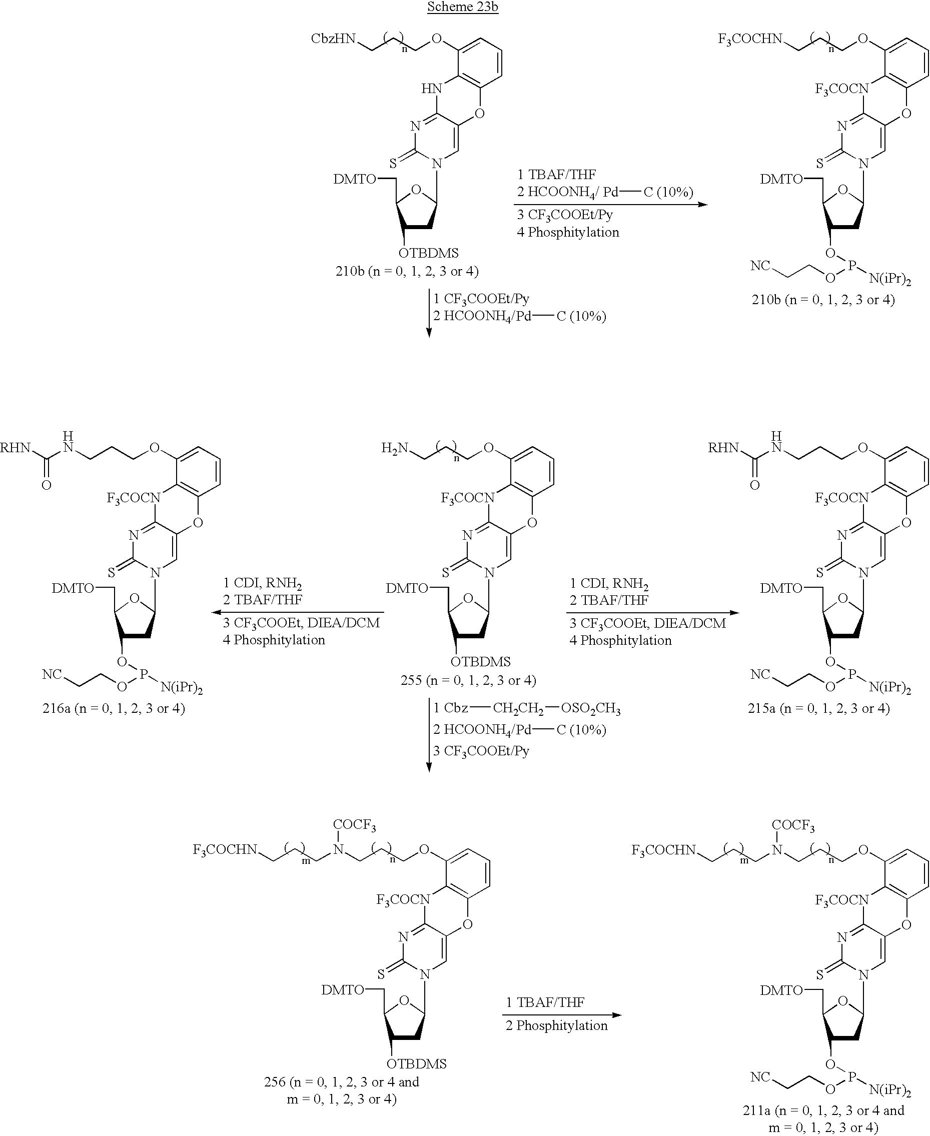

### EXAMPLE 85

### Compound 255 (n=0, Scheme 23b)

Compound 254 (n=0, 1 mmol) is stirred with ethyl trifluoroacetate (5 mmol) in pyridine at ambient temperature. The trifluoroacetamide formed after purification is stirred with ammonium formate (10 mmol) in the presence of Pd-C (10%) in ethyl acetate as described in Example 67 to obtain compound 255.

### EXAMPLE 86

### Compound 215a (n=0, R=Me, Scheme 23b, Table XXI)

Compound 255 (1 mmol) is reacted with CDI and methylamine as described in Example 68. The urea derivative thus obtained is stirred with TBAF in THF to remove 3′-O-protection. After deprotection of 3′-O-TBDMS, the resulting product is trifluoroacetylated at the ring nitrogen by stirring it with excess ethyl trifluoroacetate in anhydrous pyridine. Phosphitylation of the trifluoroacetamide derivative under the conditions described in Example 2 for the synthesis of compound 3 yields compound 215a.

### EXAMPLE 87

### Compound 216a (n=0, R=Me, Scheme 23b, Table XXI)

Compound 216a is synthesized from compound 255, 1,1′-thiocarbonyl-diimidazole and methylamine as described in Example 86 for the synthesis of compound 215a.

### EXAMPLE 88

### Compound 256 (m=0, n=0, Scheme 23b)

Compound 256 is prepared from compound 255 (1 mmol) and N-benzyloxy-carbonyl-2-aminoethanol-O-methane sulfonate (1 mmol) as described in Example 72.

### EXAMPLE 89

### Compound 211a (m=0, n=0, Scheme 23b, Table XXI)

Compound 256 is stirred with TBAF in THF to remove the TBDMS protection on the 3′-OH group. After deprotection, the 3′-OH group is phosphitylated as described in Example 2 for the synthesis of compound 3 to obtain compound 211a.

### EXAMPLE 90

### Compound 257 (n=0, Scheme 23c)

Compound 257 is obtained from compound 255 under the conditions described in Example 74.

### EXAMPLE 91

### Compound 217a (n=0, Scheme 23c, Table XXI)

Compound 217a is prepared from compound 257 as described in Example 89 for the preparation of compound 211a.

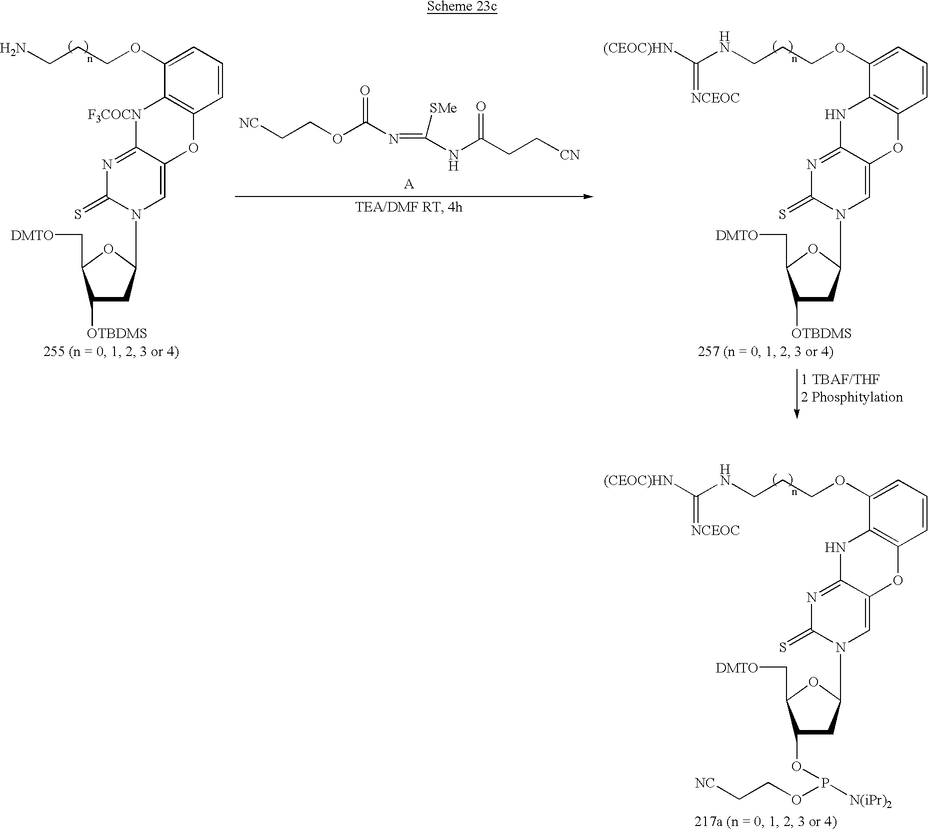

### EXAMPLE 92

### Compound 258 (n=1, Scheme 23d)

Compound 258 is synthesized from compound 252 as described in Examples 77 and 78.

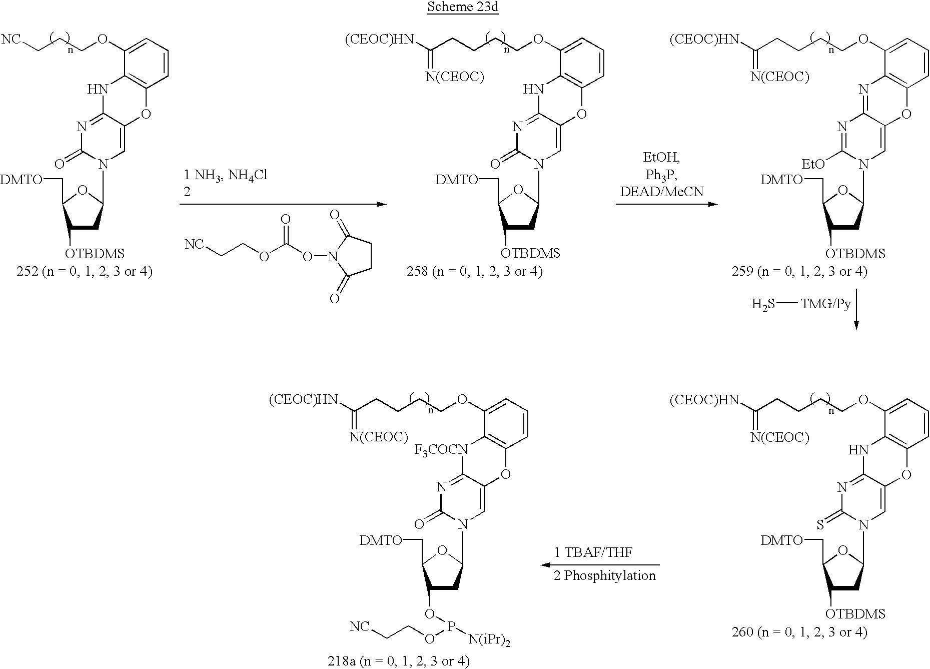

### EXAMPLE 93

### Compound 218a (n=1, Scheme 23d, Table XXI)

The phosphoramidite 218a is synthesized from compound 258 under identical conditions described in Examples 81 and 83 for the preparation of compound 210a from compound 253.

### EXAMPLE 94

### Compound 262 (Scheme 24)

2-Amino-3-methoxy-benzenethiol [Inoue et. al., Chem. Pharm. Bull., 1997, 45, 1008-1028] is reacted with Boc₂O in presence of NaHCO₃ and subsequently with Ac₂O in pyridine to obtain compound 262.

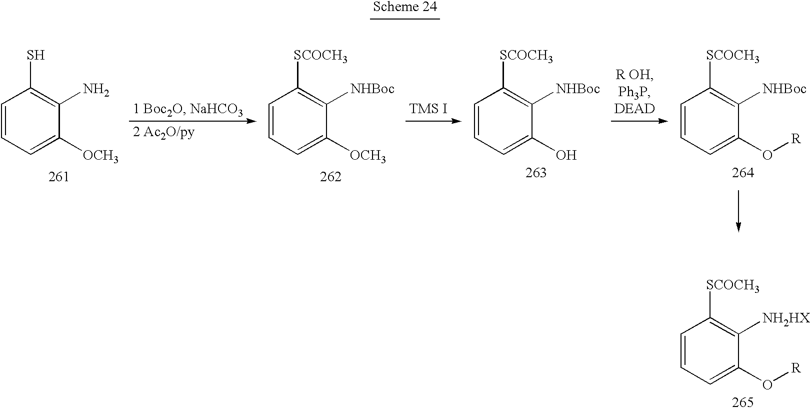

### EXAMPLE 95

### Compound 263 (Scheme 24)

After thorough drying over P₂O₅ under vacuum, compound 262 in anhydrous dichloromethane is treated with TMS-1 for 5 min to obtain compound 263.

### EXAMPLE 96

### Compound 264 (Scheme 24)

Tether of choice is attached to the hydroxyl function of compound 263 in presence of Ph₃P and DEAD (Mitsunobu alkylation) to obtain compound 264.

### EXAMPLE 97

### Compound 265 (Scheme 24)

Compound 264 is stirred with TFA in DCM for 30 min to obtain compound 265.

### EXAMPLE 98

### Compound 267 (Scheme 25a)

Compound 267 is synthesized from compound 266 and compound 265 according to reported procedures [Lin et. al., J. Am. Chem. Soc. 1995, 117, 3873-3874].

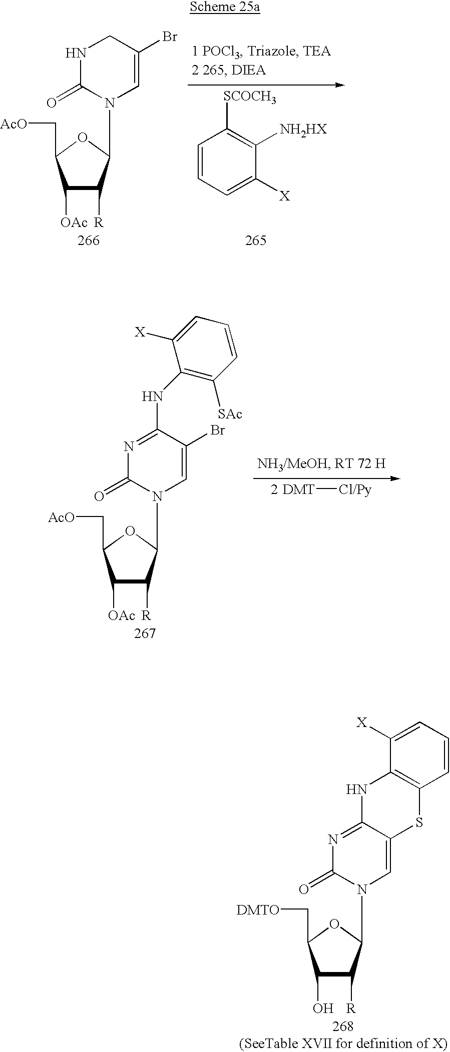

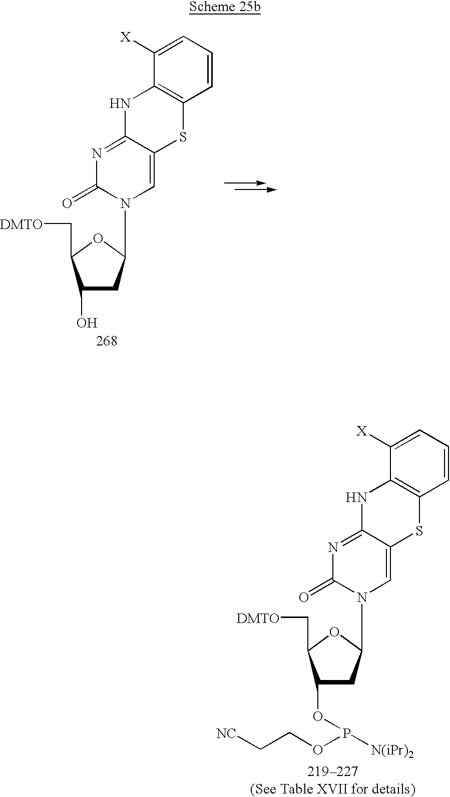

### EXAMPLE 99

### Compound 268 (Scheme 25b, Table XXII).

Tricyclic nucleoside 268 is prepared

TABLE XXII

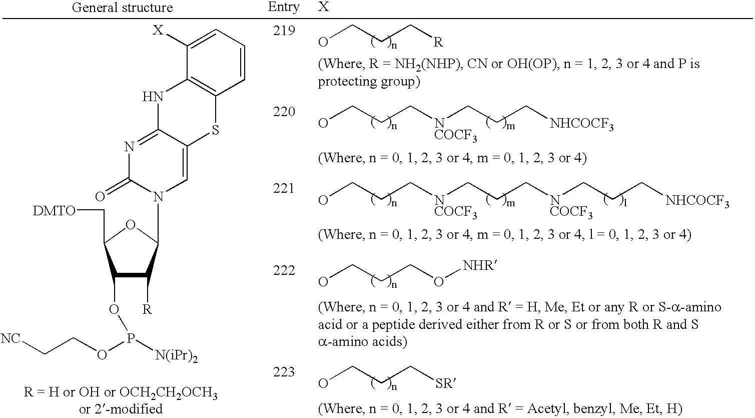

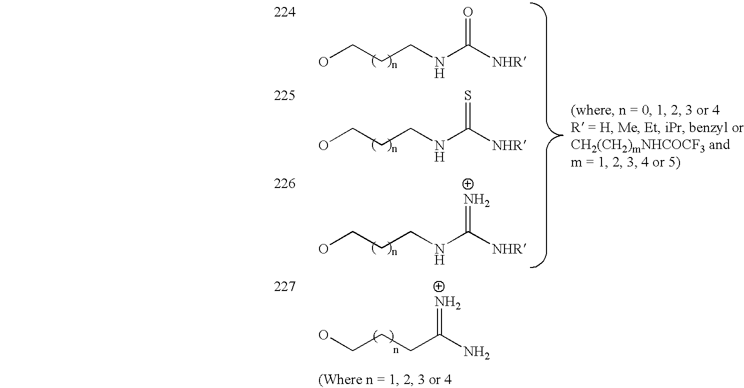

from compound 267 according to the reported procedure [Lin and Matteucci, J. Am. Chem. Soc., 1998, 120, 8531-8532].

TABLE XXIII

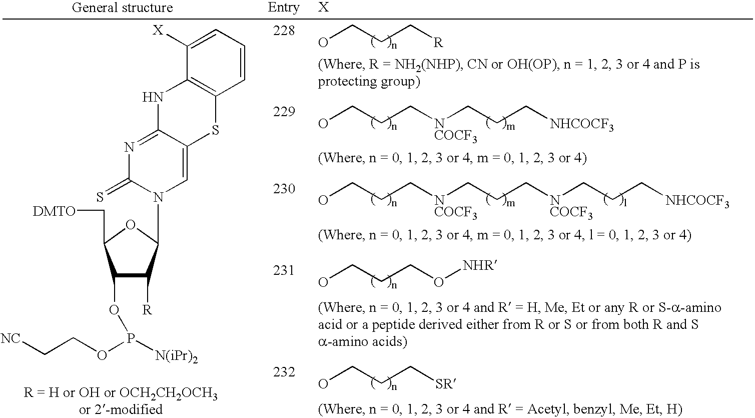

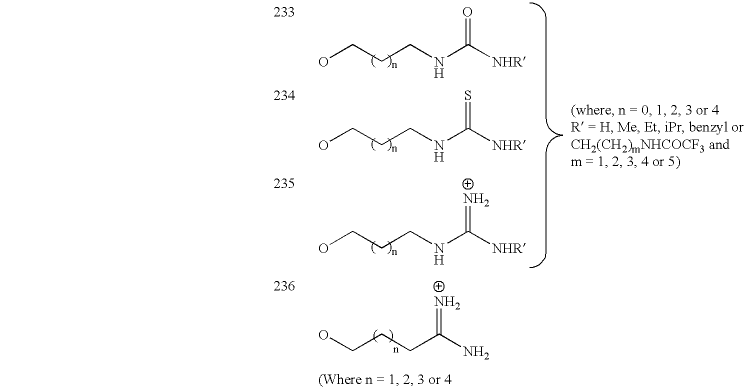

### EXAMPLE 100

### Compound 219 (X═O[CH₂]₃CN, Scheme 25b, Table XXII)

Compound 268 (X═O[CH₂]₃CN, Scheme 25a) is phosphitylated under the conditions described in Example 2 to obtain compound 219.

### EXAMPLE 101

### Compound 220 [X═O(CH₂)₂N(COCF₃)[CH₂]N(H)COCF₃], Scheme 25b, Table XXII)

Compound 220 (as specified) is prepared from compound 268 (X═O[CH₂]₂NHCbz) and N-benzyloxycarbonyl aminoethanol-O-methylsulfonate as described in Examples 67, 72 and 73.

### EXAMPLE 102

### Compound 224 (X═O[CH₂]₂NHCONHCH₃, Scheme 25b, Table XXII)

Compound 224 (as specified) is synthesized from compound 268 (X═O[CH₂]₂NHCbz), CDI and methylamine as described in Examples 67, 68 and 69.

### EXAMPLE 103

### Compound 225 (X═O[CH₂]₂NHCSNHCH₃, Scheme 25b, Table XXII)

Compound 224 (as specified) is synthesized from compound 268 (X═O[CH₂]₂NHCbz), 1,1′-thiocarbonyldiimidazole and methylamine as described in Examples 67, 70 and 71.

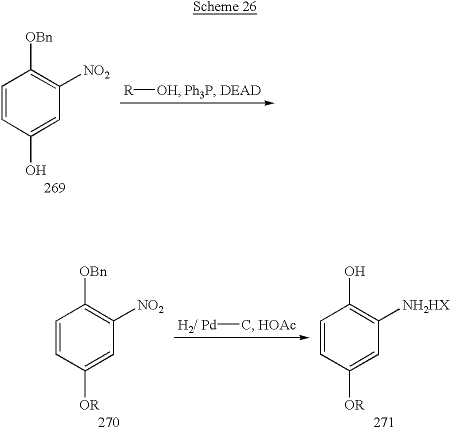

### EXAMPLE 104

### Compound 226 (X═O[CH₂]₂NHC[NH]NH₃, Scheme 25b, Table XXII)

Compound 226 (as specified) is synthesized from compound 268 (X═O[CH₂]₂NHCbz) and compound A (See Scheme 22d) as described in Examples 67, 74 and 75.

### EXAMPLE 105

### Compound 227 (X═O[CH₂]₃CH₂C[NH]NH₃, Scheme 25b, Table XXII)

Compound 227 (as specified) is synthesized from compound 268 (X═O[CH₂]₃CN) as described in Examples 77, 78 and 79.

### EXAMPLE 106

### Compound 270 (Scheme 26)

Alkylation of hydroxyl function of compound 269 [Bigge, C. F. et. al., PCT Int. Appl. (1997), 280 pp CODEN PIXXD2 WO 9723216 A1 19970703] using tether of choice (as defined in Table XXIV) is presence of Ph₃P and DEAD yields compound 270.

### EXAMPLE 107

### Compound 271 (Scheme 26)

Compound 271 (1 mmol) is dissolved in ethyl acetate containing 10% acetic acid, the resulting solution after deoxygenation is mixed with 10 mol percentage of pd-C (10%) subjects to catalytic hydrogenation under pressure to obtain compound 271.

### EXAMPLE 108

### Compound 273 (Scheme 27a)

Compound 272 is obtained from compound 266 and compound 271 as described in Examples 88 and 98.

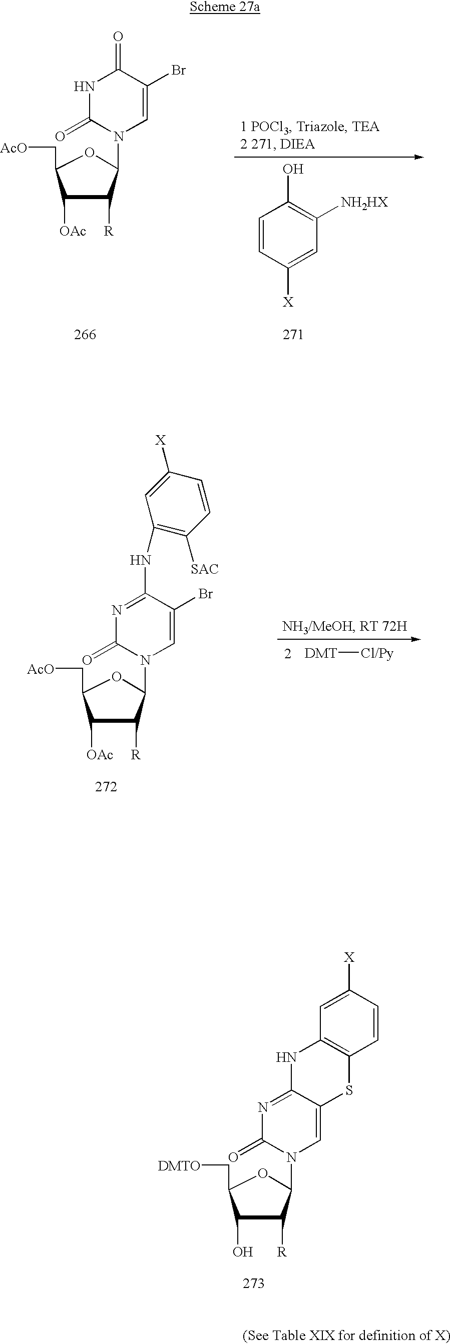

### EXAMPLE 109

### Compound 237 (X═O[CH₂]₂N(Phtaloyl), Scheme 27b, Table XXIV)

Phosphitylation of compound 273 (X═O[CH₂]₂N(Phthaloyl), Scheme 27a) under identical conditions described in Example 2 yields compound 237.

TABLE XXIV

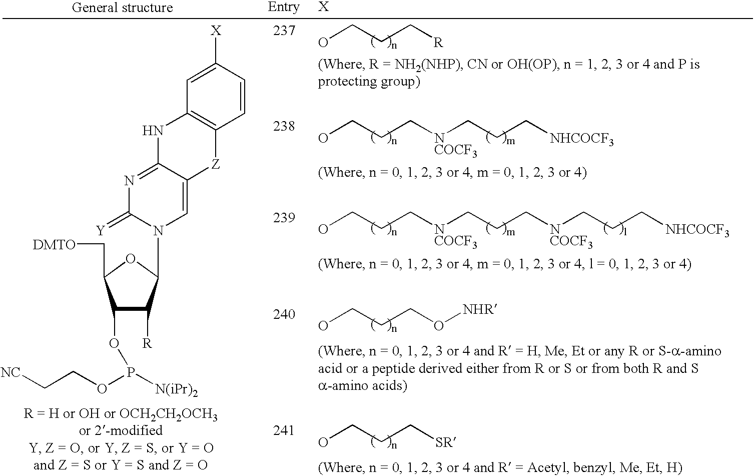

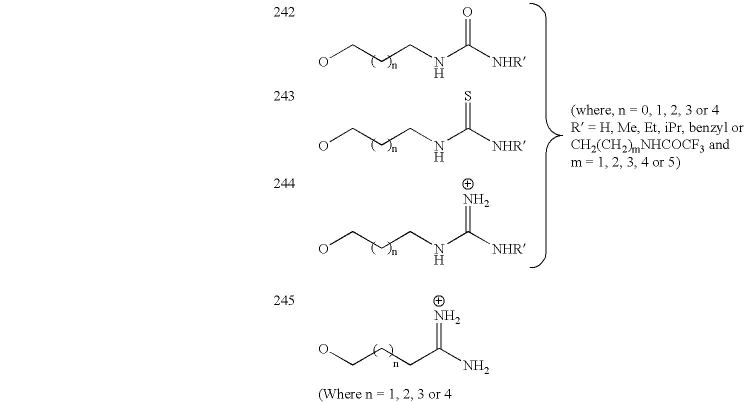

### EXAMPLE 110

### Compound 238 (X═O[CH₂]₂N{COCF₃}[CH₂]₂NH{COCF₃}, Scheme 27b, Table XXIV)

Compound 273 (X═O[CH₂]₂N {Phthaloyl}, Scheme 27a) is treated with hydrazine to remove the phthaloyl protection from the side chain. The corresponding free amine thus formed is reacted with N-benzyloxycarbonyl aminoethanol-O-methane sulfonate in presence of base as described in Example 64, followed by phosphitylation (Example 2) yields compound 238.

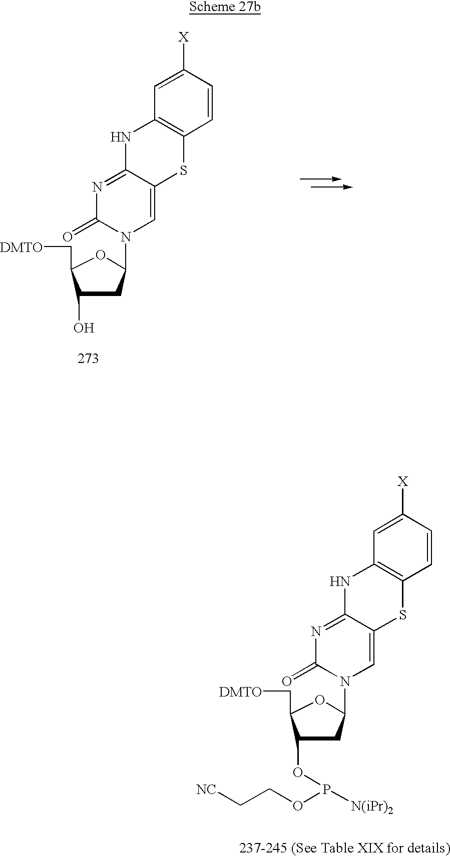

### EXAMPLE 111

### Compound 242 (X═O[CH₂]₂NHCONHCH₃, Scheme 27b, Table XXII)

Compound 273 (X═O[CH₂]₂N {Phthaloyl}, Scheme 27a) is treated with hydrazine to remove the phthaloyl protection from the side chain. The desired compound 242 is obtained by reacting the free amino group formed with CDI and methylamine as described in Examples 68 and 69.

### EXAMPLE 112

### Compound 243 (X═O[CH₂]₂NHCSNHCH₃, Scheme 27b, Table XXII)

Compound 273 (X═O[CH₂]₂N {Phthaloyl}, Scheme 27a is treated with hydrazine to remove the phthaloyl protection from the side chain. The desired compound 243 is obtained by reacting the free amino group formed with 1,1′-thiocarbonyldiimidazole and methylamine as described in Examples 68 and 69.

### EXAMPLE 113

### Compound 244 (X═O[CH₂]₂NHC{NH}NH₃, Scheme 27b, Table XXII)

Compound 273 (X═O[CH₂]₂N {Phthaloyl}, Scheme 27a) is treated with hydrazine to remove the phthaloyl protection from the side chain. The desired compound 243 is prepared from the amino compound and compound A (See Scheme 22d) as described in Examples 67, 74 and 75.

### EXAMPLE 114

### Compound 245 (X═O[CH₂]₃CH₂C[NH]NH₃, Scheme 27b, Table XXII)

Compound 227 (as specified) is synthesized from compound 273 (X═O[CH₂]₃CN) as described in Examples 77, 78 and 79.

### EXAMPLE 115

### Compound 284 (Scheme 28)

Compound 283 prepared according to the literature procedure [Pal, B, C. et al., Nucleosides & Nucleotides, 1988, 7, 1-21] is stirred with Boc₂O in presence of NaHCO₃ in aqueous methanol to protect the ring nitrogen as Boc. The Boc protected nucleoside is then acetylated in anhydrous pyridine to obtain compound 284.

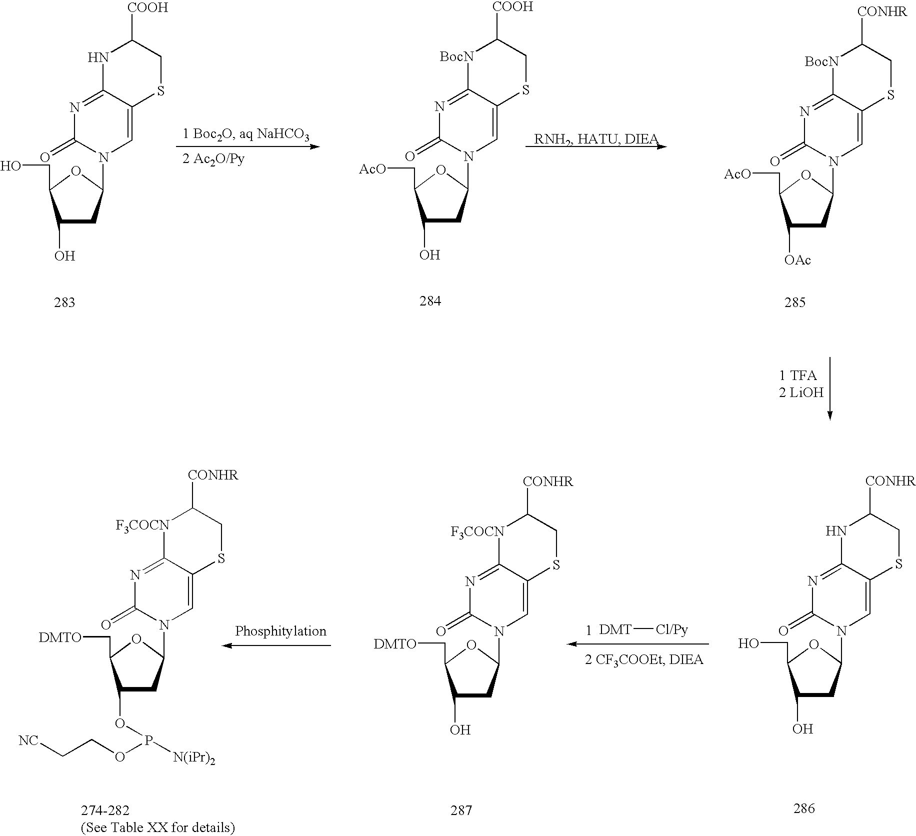

### EXAMPLE 116

### Compound 285 (R=[Phthaloyl]N[CH₂]₃—, Scheme 28)

N-(Phthaloyl)ethylenediamine is coupled to the carboxyl group of compound 284 in the presence of HATU and HOAT under peptide coupling conditions to obtain compound 285.

### EXAMPLE 117

### Compound 286 (R=[Phthaloyl]N[CH₂]₃—, Scheme 28)

Compound 285 is subjected to TFA treatment in dichloromethane for 30 min to remove the Boc protection. After deblocking the ring nitrogen, the resulting compound is stirred in aqueous THF containing 0.1 M LiOH at 0° C. to obtain compound 286 (as specified).

### EXAMPLE 118

### Compound 287 (R=[Phthaloyl]N[CH₂]₃—, Scheme 28)

Compound 287 (1 mmol) in anhydrous pyridine is treated with DMT-Cl (1 mmol) in presence of DMAP (10 mol %) to obtain the corresponding 5′-O-DMT derivative. After dimethoxytritylation, the resulting product is stirred with excess of ethyl trifluoroacetate in presence of DIEA in anhydrous dichloromethane to obtain compound 287.

### EXAMPLE 119

### Compound 274 (R=[Phthaloyl]N[CH₂]₃—, Scheme 28, Table XXV)

Phosphitylation of compound 287 under the conditions described in Example 2 for the synthesis of compound 3 yields compound 274.

TABLE XXV

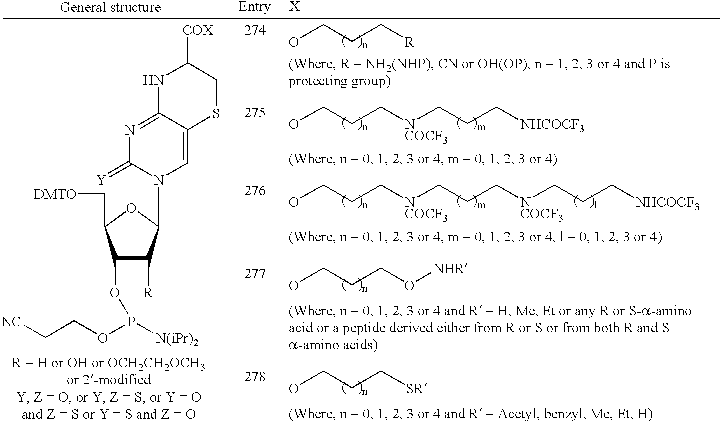

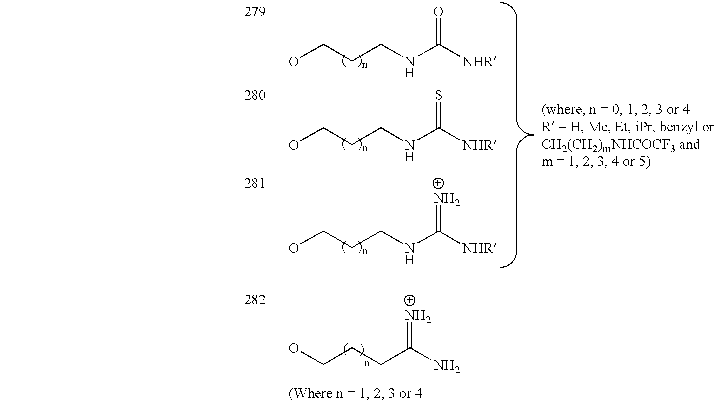

### EXAMPLE 120

### Nuclease Resistance of Oligonucleotides with Selected Modifications

Phenoxazine 151 and G-clamp 152 nucleosides were prepared by modifying previously published procedures [Lin, K. -Y.; Jones, R. J.; Matteucci, M. J. Am. Chem. Soc. 1995, 117, 3873-3874; Lin, K. -Y.; Matteucci, M. J. Am. Chem. Soc. 1998, 120, 8531-8532]. The succinates 153 and 154 and the corresponding substituted solid supports 155 and 156 were prepared as outline in Scheme 19. Using the CPG supports, the two cytidine analogs 151 and 152 were incorporated at the 3′-terminus of two model oligonucleotides 157 and 158, respectively, with the sequence T₁₈dC* (dC*=phenoxazine (SEQ ID NO:62) or G-clamp deoxyribonucleoside (SEQ ID NO:63). Solid phase oligonucleotide syntheses was carried out using standard phosphoramidite chemistry. Deprotection of G-clamp containing oligonucleotides 158 was performed with a 1:1 solution of MeNH₂ (40%, aq.) and NH₃ (28-30 aq.) at r.t. for 4 h. The oligonucleotides were purified and desalted by reversed phase HPLC.

Snake venom phosphodiesterase (SVPD) and bovine intestinal mucosal phosphodiesterase (BIPD), were utilized as the hydrolytic enzymes for in vitro nuclease resistance studies. Both enzymes predominantly exhibit 3′ exonuclease activity. An unmodified 19 mer oligothymidylate (oligonucleotide 159) (SEQ ID NO:64) was used as a control. Oligonucleotide samples were incubated with SVPD (2.5 units/μmol substrate) or BIPD (0.55 units/μmol substrate) in 50 mM Tris-HCl, 8 mM MgCl₂ buffer, pH 7.5 at 37° C. At certain time points aliquots of 10 μl were removed and heated in boiling water for 2 min to inactivate the enzyme. Subsequently, the samples were desalted by membrane dialysis against Nanopure deionized water using Millipore 0.025 μm VS membranes and stored frozen until they were analysed. The progress of enzymatic degradation was monitored by capillary gel electrophoresis (CGE).

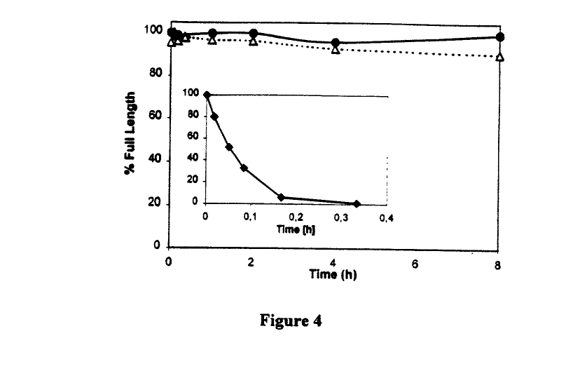

The results of the nuclease resistance study with SVPD as the hydrolytic enzyme are shown in FIG. 4. As expected, the unmodified control oligonucleotide 159 (insert) was degraded rapidly by sequential removal of the terminal nucleotides. Under the applied conditions the t_{½} for this oligonucleotide was reached at about 3 min. After 20 min of incubation the full length oligomer was almost completely degraded to a series of shorter fragments. In contrast, the modified oligonucleotides 157 and 158 bearing the heterocyclic modifications at their 3′ end were not significantly degraded even after an incubation time of 8 h. According to the degradation rates and the CGE profiles, there is no significant difference in the 3′ exonuclease resistance of these two oligomers. Very similar results for the nuclease resistance against BIPD as the hydrolytic enzyme were obtained for both modified oligonucleotides 157 and 158.

In a second set of experiments, the inhibitory effects of phenoxazine and G-clamp oligonucleotides on the nuclease activity was investigated. Unmodified oligonucleotide 159 was incubated with BIPD and the degradation of a 19 mer oligothymidylate with 5′ labeled with fluorescein was followed under the presence of various amounts of oligonucleotides 157 and 158, respectively. Olignucleotide samples were incubated with BIPD (0.55 units/μmol substrate) in 50 mM Tris-HCl, 8 mM MgCl₂, pH 7.5 at 37° C. At certain time points aliquots of 10 μl were withdrawn and diluted directly into 200 μL dH2O before CGE analysis. The influence of the modified oligonucleotides on the nucleolytic activity was determined by looking at the overall velocity of the enzymatic reaction. Therefore, all products of degradation were quantified at each time point, weighted considering their stage of degradation (n−x) and summarized to obtain the number of hydrolyzed linkages. The velocity of the enzymatic reaction was determined graphically from the number of hydrolyzed phosphodiester linkages as a function of the incubation time.

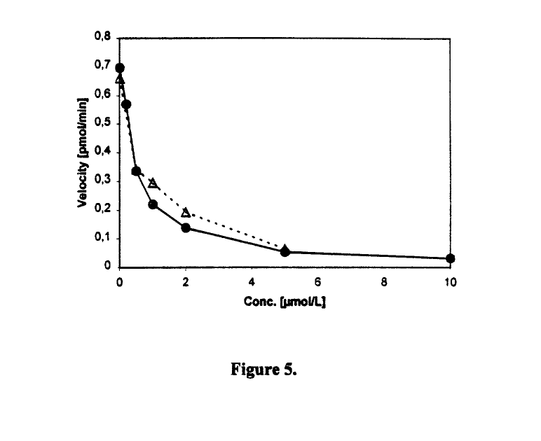

This second part of our study was driven by the question why oligonucleotides bearing these tricyclic base modifications at their 3′ terminus exhibit such extraordinary nuclease resistance. Therefore it was intended to determine whether or not they are recognized as a substrate, i.e. whether or not they are bound to the active site of the enzyme and are capable to affect the degradation of a natural DNA fragment. In FIG. 5, the velocity of the enzymatic degradation of unmodified oligonucleotide 159 is depicted as a function of the concentration of oligonucleotide 157 and 158. From the diagram it is obvious that the presence of the modified oligonucleotides has a distinct inhibitory effect on the enzymatic reaction. Again, no significant difference is detectable between the two derivatives phenoxazine and G-clamp. Both are capable to slow down the degradation process of oligonucleotide 159 at concentrations above 0.2 μM. The IC50 values are reached at about 0.5 μM and at concentrations of 5 μM and higher the enzymatic reaction is almost completely prohibited.

### EXAMPLE 120

### Nuclease Resistance of Oligonucleotides with Selected Modifications

Phenoxazine 151 and G-clamp 152 nucleosides were prepared by modifying previously published procedures [Lin, K. -Y.; Jones, R. J.; Matteucci, M. J. Am. Chem. Soc. 1995, 117, 3873-3874; Lin, K. -Y.; Matteucci, M. J. Am. Chem. Soc. 1998, 120, 8531-8532]. The succinates 153 and 154 and the corresponding substituted solid supports 155 and 156 were prepared as outlined in Scheme 19. Using the CPG supports, the two cytidine analogs 151 and 152 were incorporated at the 3′ terminus of two model oligonucleotides 157 and 158, respectively, with the sequence T₁₈dC* (dC*=phenoxazine (SEQ ID NO:62) or G-clamp deoxyribonucleoside (SEQ ID NO:63)). Solid phase oligonucleoside synthesis was carried out using standard phosphoramidite chemistry. Deprotection of G-clamp containing oligonucleotide 158 was performed with a 1:1 solution of MeNH₂ (40%, aq.) and NH₃ (28-30%, aq.) at r.t. for 4 h. The oligonucleotides were purified and desalted by reversed phase HPLC.

Snake venom phosphodiesterase (SVPD) and bovine intestinal mucosal phosphodiesterase (BIPD), were utilized as the hydrolytic enzymes for in vitro nuclease resistance studies. Both enzymes predominantly exhibit 3′ exonuclease activity. An unmodified 19 mer oligothymidylate (oligonucleotide 159) (SEQ ID NO:64) was used as a control. Oligonucleotide samples were incubated with SVPD (2.5 units/μmol substrate) or BIPD (0.55 units/μmol substrate) in 50 mM Tris-HCl, 8 mM MgCl₂ buffer, pH 7.5 at 37° C. At certain time points aliquots of 10 μl were removed and heated in boiling water for 2 min to inactivate the enzyme. Subsequently, the samples were desalted by membrane dialysis against Nanopure deionized water using Millipore 0.025 μm VS membranes and stored frozen until they were analysed. The progress of enzymatic degradation was monitored by capillary gel electrophoresis (CGE).

The results of the nuclease resistance study with SVPD as the hydrolytic enzyme are shown in FIG. 4. As expected, the unmodified control oligonucleotide 159 (insert) was degraded rapidly by sequential removal of the terminal nucleotides. Under the applied conditions the t_{½} for this oligonucleotide was reached at about 3 min. After 20 min of incubation the full length oligomer was almost completely degraded to a series of shorter fragments. In contrast, the modified oligonucleotides 157 and 158 bearing the heterocyclic modifications at their 3′ end were not significantly degraded even after an incubation time of 8 h. According to the degradation rates and the CGE profiles, there is no significant difference in the 3′ exonuclease resistance of these two oligomers. Very similar results for the nuclease resistance against BIPD as the hydrolytic enzyme were obtained for both modified oligonucleotides 157 and 158.

In a second set of experiments, the inhibitory effects of phenoxazine and G-clamp oligonucleotides on the nuclease activity was investigated. Unmodified oligonucleotide 159 was incubated with BIPD and the degradation of a 19 mer oligothymidylate with 5′ labeled with fluorescein was followed under the presence of various amounts of oligonucleotides 157 and 158, respectively. Oligonucleotide samples were incubated with BIPD (0.55 units/μmol substrate) in 50 mM Tris-HCl, 8 mM MgCl₂, pH 7.5 at 37° C. At certain time points aliquots of 10 μl were withdrawn and diluted directly into 200 μL dH2O before CGE analysis. The influence of the modified oligonucleotides on the nucleolytic activity was determined by looking at the overall velocity of the enzymatic reaction. Therefore, all products of degradation were quantified at each time point, weighted considering their stage of degradation (n−x) and summarized to obtain the number of hydrolyzed linkages. The velocity of the enzymatic reaction was determined graphically from the number of hydrolyzed phosphodiester linkage as a function of the incubation time.

The second part of our study was driven by the question why oligonucleotides bearing these tricyclic base modifications at their 3′ terminus exhibit such extraordinary nuclease resistance. Therefore it was intended to determine whether or not they are recognized as a substrate, i.e. whether or not they are bound to the active site of the enzyme and are capable to affect the degradation of unmodified oligonucleotide 159 is depicted as a function of the concentration of oligonucleotide 157 and 158. From the diagram it is obvious that the presence of the modified oligonucleotides has a distinct inhibitory effect on the enzymatic reaction. Again, no significant difference is detectable between the two derivatives phenoxazine and G-clamp. Both are capable to slow down the degradation process of oligonucleotide 159 at concentration above 0.2 μM. The IC50 values are reached at about 0.5 μM and at concentrations of 5 μM and higher the enzymatic reaction is almost completely prohibited.

The nuclease resistance data demonstrate that, despite their natural phosphodiester backbones, both heterocyclic modifications provide an almost complete protection against 3′ exonuclease attack. Obviously the enzyme is not capable to digest oligonucleotides, which contain the modified nucleobases phenoxazine or G-clamp at their 3′ terminus. The observed high nuclease stability could principally have various reasons. Either the bulky heterocycle moieties simply prevent the enzyme from binding to the 3′-terminus by steric hindrance, meaning that the oligonucleotides are not recognized as a substrate, or they bind to the active site of the enzyme without being hydrolyzed, which would directly affect the enzyme's activity. The observed decrease in the velocity of the enzymatic degradation of a natural DNA fragment indicates that oligonucleotides containing phenoxazine and G-clamp residues are able to bind to the enzyme's active site. Hydrolysis of the 3′ terminal nucleotide phosphodiester linkage, however, is prevented due to the presence of the unnatural tricyclic base moieties. The dose-dependence of the inhibitory effects with IC50 values of about 0.5 μMol suggests that the binding of the modified oligonucleotides is competitive and reversible.

There is not detectable difference between the nuclease resistance of oligonucleotides 157 and 158 indicating that the observed stabilizing effect is mainly due to presence of the bulky heterocycles. With the present data, however, it remains unclear to what extent the positively charged amino tether of the G-clamp moiety contributes to the nuclease resistance of oligonucleotide 158. In previous studies it has been shown that cationic modifications of the sugar moieties, such as 2′-O-aminoalkyl, can efficiently protect phosphodiester oligonucleotides from enzymatic degradation [Manoharan, M.; Tivel, K. L.; Anrade, L. K., Cook, P. D. Tetrahedron Lett. 1995, 36, 3647-3650; Teplova, M.; Wallace, S. C.; Tereshko, V.; Minasov, G.; Symons, A. M.; Cook, P. D.; Manoharan, M.; Egli, M. PNAS 1999, 96, 14240-14245]. Crystal structure studies of a complex formed between a 2′-aminopropyl modified oligonucleotide and an exonuclease (DNA polymerase I Klenow fragment) demonstrate that the aminopropyl residue prevents binding of a metal ion, which is needed to catalyze hydrolysis of the 3′ phosphodiester linkage. The amino tether of a G-clamp residue, however, protrudes into the major groove, while the 2′ modification points into the shallow groove of a duplex. Whether or not the positive charge of the latter can interfere with the metal binding of an exonuclease remains to be investigated.

### EXAMPLE 121

### Degradation by SVPD

Oligonucleotides, at a final concentration of 2 μM, were incubated with snake venom phosphodiesterase (0.005 U/ml) in 50 mM Tris-HCl, pH 7.5, 8 mM MgCl₂ at 37° C. The total reaction volume was 100 μL. At each time point 10 μL aliquots of each reaction mixture were placed in a 500 μL microfuge tube and put in a boiling water bath for two minutes. The sample was then cooled on ice, quick spun to bring the entire volume to the bottom of the tube, and desalted on a Millipore 0.025 micron filter disk (Bedford, Mass.) that was floating in water in a 60 mm petrie dish. After 30-60 minutes on the membrane the sample was diluted with 200 μL distilled H₂O and analyzed by gel-filled capillary electrophoresis. The oligonucleotide and metabolites were separated and analyzed using the Beckman P/ACE MDQ capillary electrophoresis instrument using a 100 μm ID 30 cm coated capillary (Beckman No. 477477) with eCAP ssDNA 100-R gel (Beckman No. 477621) and Tris-Borate Urea buffer (Beckman No. 338481). The samples were injected electrokinetically using a field strength of between 5-10 kV for a duration of between 5 and 10 seconds. Separation wash achieved at 40° C. with an applied voltage of 15 kV. The percentage of full length oligonucleotide was calculated by integration using Caesar v.6 software (Senetec Software, New Jersey) followed by correction for differences in extinction coefficient for oligonucleotides of different length.

### EXAMPLE 122

### In Vivo Nuclease Stability and Binding Affinity Properties of L/S-oligonucleotide Chimera

Naturally occurring D-Oligonucleotides are degraded by nucleases very rapidly whereas enatiomeric L-DNA oligomers have enhanced resistance to the action of nucleases¹. However L-DNA have been found to hybridize either weakly or not at all with natural RNA and DNA. Damha and Capobianco [Damha, M. J.; Giannaris, P. A., Marfey, P. Biochemistry, 1994, 33, 7877-7885; Capobinaco, m. L.; Garbesi, A.; Arcamone, F.; Maschera, B.; Palu, G. Nucleic Acids Symp. Series 1991, 24, 274] independently have shown that chimeric L/D-oligomers with terminal L-units provided adequate duplex forming capability and excellent enzymatic stability in human serum [Damha, M. J.; Giannaris, P. A., Marfey, P. Biochemistry, 1994, 33, 7877-7885; Capobinaco, m. L.; Garbesi, A.; Arcamone, F.; Maschera, B.; Palu, G. Nucleic Acids Symp. Series 1991, 24, 274].

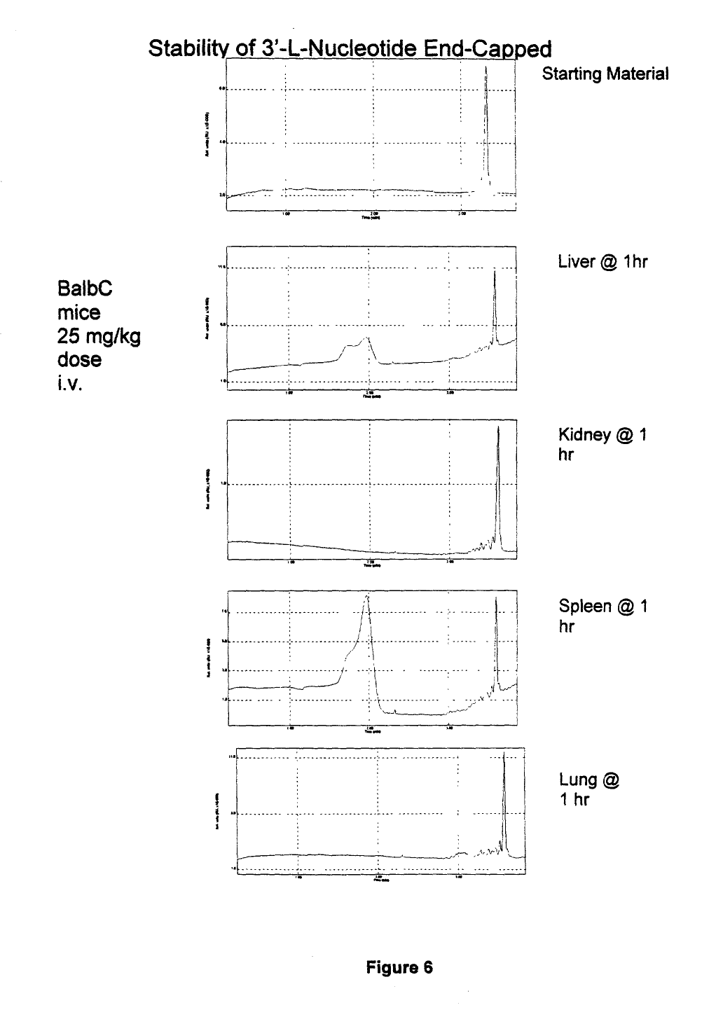

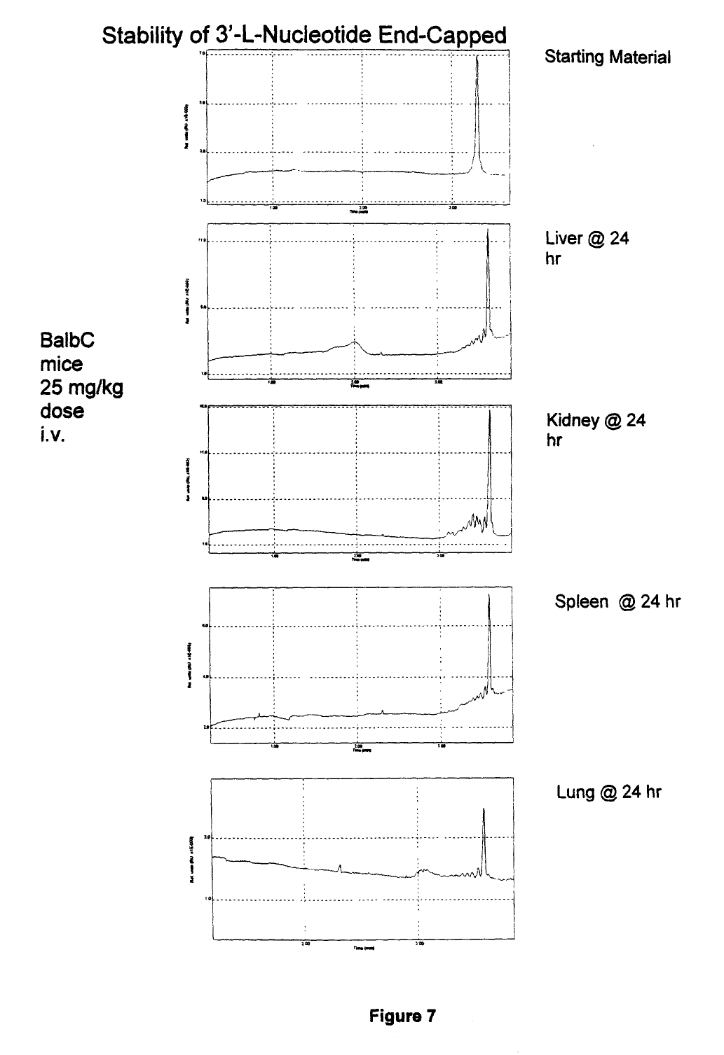

Here we report the in vivo nuclease stability of L/D-oligonucleotide chimera in mouse. We synthesized the phosphoramidite and CPG derived from L-thymidine, which was synthesized from a novel route [Jung, E. M.; Xu, Y. Tetrahedron Lett. 1997, 24, 4199-4202]. A 20 mer phosphorothioate oligonucleotide ISIS-120745 (antisense to mouse ICAM-1) was capped with L-2′-deoxy thymidine at 3′ and 5′-positions. The oligonucleotide was then administered IV bolus into BalbC mouse. After 24 h. mouse was sacrificed and the oligonucleotide was isolated from different organs. Percentage of full-length oligonucleotide present in different organs were analyzed by CGE. From all the major organs >90% of the intact L-thymidine capped oligonucleotide was isolated where as the parent oligonucleotide was degraded completely (FIGS. 6 and 7).

What is claimed is:

1. A method of enhancing the nuclease resistance of an oligonucleotide comprising preparing said oligonucleotide having at least one modified nucleoside at either the 3′ or the 5′-terminus wherein said nucleoside comprises a tricyclic heterocyclic base moiety thereon.

2. The method of claim 1 wherein said modified nucleoside is at the 5′-terminus of said oligonucleotide.

3. The method of claim 1 wherein said modified nucleoside is at the 3′-terminus of said oligonucleotide.

4. The method of claim 1 wherein said oligonucleotide having enhanced nuclease resistance is of the formula: 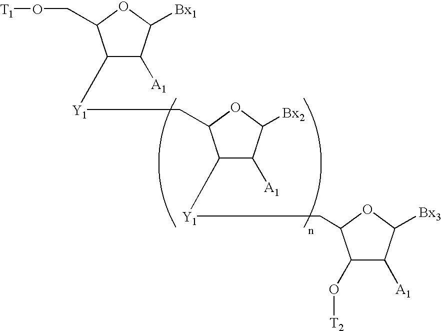
wherein:
each Y₁ is, independently, an internucleoside linking group;
each of Bx₁, Bx₂ and Bx₃ is a—ensure that the definition of heterocyclic base moiety includes optionally protected in the detailed description—heterocyclic, base moiety wherein at least one of Bx₁ and Bx₃ is a tricyclic heterocyclic base moiety;
each A₁ is, independently, hydrogen or a 2′-substituent group;
T₁ is hydrogen or a hydroxyl protecting group;
T₂ is hydrogen or a hydroxyl protecting group; and
n is from 2 to about 50.

5. The method of claim 4 wherein Bx₁ is a tricyclic heterocyclic base moiety.

6. The method of claim 4 wherein Bx₃ is a tricyclic heterocyclic base moiety.

7. The method of claim 4 wherein each of said tricyclic heterocyclic base moieties is of the formula: 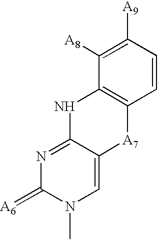
wherein
A₆ is O or S;
A₇ is CH₂, NCH₃, O or S;
each A₈ and A₉ is hydrogen or one of A₈ and A₉ is hydrogen and the other of A₈ and A₉ is selected from the group consisting of:
—O—(CH₂)ₚ₁—G₁
and 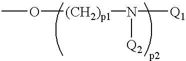
wherein
G₁ is —CN, —OA₂₀, —SA₂₀, —N(H)A₂₀, —ON(H)A₂₀ or —C(═NH)N(H)A₂₀,
Q₁ is H, —NHA₂₀, —C(═O)N(H)A₂₀, —C(═S)N(H)A₂₀ or —C(═NH)N(H)A₂₀,
each Q₂ is, independently, H or Pg;
A₂₀ is H, Pg, substituted or unsubstituted C₁-C₁₀ alkyl, acetyl, benzyl, —(CH₂)ₚ₃NH₂, —(CH₂)ₚ₃N(H)Pg, a D or L α-amino acid, or a peptide derived from D, L or racemic α-amino acids;
Pg is a nitrogen, oxygen or thiol protecting group;
each p1 is, independently, from 2 to about 6,
p2 is from 1 to about 3; and
p3 is from 1 to about 4.

8. The method of claim 7 wherein:
A₆ is O or S;
A₇, if O or S;
A₉ is H;
A₈ is —O—(CH₂)₂—N(H)A₂₁, —O—(CH₂)₂—ON(H)A₂₁ or —O—(CH₂)₂—C(═NH)N(H)A₂₁, —O (CH₂)₃—C(═NH)N(H)A₂₁, —O—(CH₂)₂—C(═O)N(H)A₂₁, —O— (CH₂)₂—C(═S)N(H)A₂₁ or —O—(CH₂)₂—N(H)C(═NH)N(H)A₂₁; and
A₂₁ is hydrogen or an amino protecting group.

9. The method of claim 8 wherein A₆ is O.

10. The method of claim 8 wherein A₆ and A₇ are both O.

11. The method of claim 4 wherein each of said internucleoside linking groups is a phosphorus-containing internucleoside linking group.

12. The method of claim 11 wherein each of said phosphorus containing internucleoside linking group is selected from the group consisting of phosphodiester, phosphorothioate, chiral phosphorothioate, phosphorodithioate, phosphotriester, aminoalkylphosphotriester, methyl phosphonate, alkyl phosphorate, 5′-alkylene phosphonate, chiral phosphonate, phosphinate, phosphoramidate, 3′-amino phosphoramidate, aminoalkylphosphoramidate, thionophosphoramidate, thionoalkylphosphonate, thionalkylphosphotriester, selenophosphate and boranophosphate.

13. The method of claim 4 wherein greater than 90% of said internucleoside linking groups are phosphodiester internucleoside linking groups.

14. The method of claim 4 wherein at least on of said internucleoside linking groups is a non-phosphorus containing internucleoside linking group.

15. The method of claim 14 wherein greater than 90% of said internucleoside linking groups are non-phosphorus containing internucleoside linking groups.

16. The method of claim 15 wherein each of said non-phosphorus containing internucleoside linking groups is, independently, selected from the group consisting of morpholi 2no, siloxane, sulfide, sulfoxide, sulfone, formacetyl, thioformacetyl, methylene formacetyl, thioformacetyl, sulfamate, methyleneimino, methylenehydrazino, sulfonate, sulfonamide, and amide.

17. The method of claim 16 wherein each of said non-phosphorus containing internucleoside linking groups is, independently, selected from the group consisting of CH₂—NH—O—CH₂—, —CH₂—N(CH₃)—O—CH₂, —CH₂—O—N(CH₃)—CH₂—, —CH₂—N(CH₃)— N(CH₃)—CH₂— and —O—N(CH₃)—CH₂—CH₂—.

18. The method of claim 4 wherein said oligomeric compound is a gapmer, hemimer or inverted gapmer.

19. The method of claim 18 wherein said oligomeric compound comprises at least one 2′—O—CH₂CH₂—O—CH₃ substituent group in at least one region of said gapmer, hemimer or inverted gapmer.

20. The method of claim 4 wherein n is from about 8 to about 30.

21. The method of claim 4 wherein n is from about 15 to about 25.

22. The method of claim 4 wherein each of said Bx₁ and Bx₃ that is not a tricyclic heterocyclic base moiety and each of said Bx₂ is, independently, selected from the group consisting of adeninyl, guaninyl, thyminyl, cytosinyl, uracilyl, 5-methylcytosinyl (5-me-C), 5-hydroxymethyl cytosinyl, xanthinyl, hyposanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 6-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

23. The method of claim 1 wherein each group sugar substituent group is, independently, C₁-C₂₀ alkyl, C₂-C₂₀ alkenyl, C₂-C₂₀ alkynyl, C₅-C₂₀ aryl, —O-alkyl, —O—alkenyl, —O—alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-aklenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—R), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—R), sulfone (—S(═O)₂—R), disulfide (—S—S—R), silyl, heterocyclyl, carboxyclyl, an intercalator, a reporter group, a conjugate group, polyamine, polyamide, polyalkylene glycol or a polyether of the formula (—O-alkyl)ₘ, where m is 1 to about 10;
wherein each R is, independently, hydrogen, a protecting group or substituted or unsubstituted alkyl, alkenyl, or alkynyl wherein the substituent groups are selected from haloalkyl, alkenyl, alkoxy, thioalkoxy, haloalkoxy or aryl as well as halogen, hydroxyl, amino, azido, carboxy, cyano, nitro, mercapto, a sulfide group, a sulfonyl group and a sulfoxide group;
or each sugar substituent group has one of formula I or II: 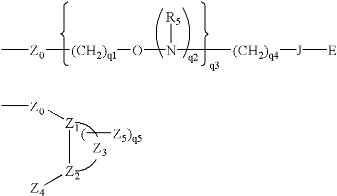
wherein
Z₀ is O, S or NH;
J is a single bond, O or C(═O);
E is C₁-C₁₀ alkyl, N(R₅)(R₆), N(R₅)(R₇), N═C(R₅ₐ)(R₆ₐ), N═C(R₅ₐ)(R₇ₐ) or has one of formula III or IV; 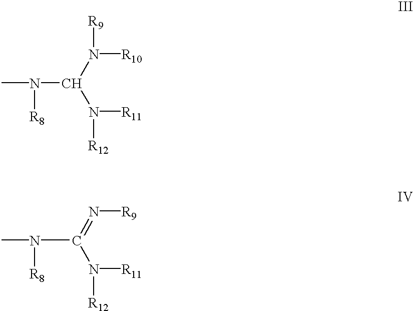
each R₈, R₉, R₁₀, R₁₁ and R₁₂ is, independently, hydrogen, C(O)R₁₃, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, R₉ and R₁₀, together from a phthalimido moiety with the nitrogen atom to which they are attached;
or optionally, R₁₁ and R₁₂, together form a phthalimido moiety with the nitrogen atom to which they are attached;
each R₁₃ is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
R₅ is hydrogen, a nitrogen protecting group or —T—L,
R₅ₐ is hydrogen, a nitrogen protecting group or —T—L,
T is a bond or a linking moiety;
L is a chemical functional group, a conjugate group or a solid support medium;
each R₆ and R₇ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, carboxy, benzyl, phenyl, nitro, thiol, thioalkoxy, halogen, alkyl, aryl, alkenyl, alkynyl; NH₃⁺, N(R₁₄)(R₁₅), guanidino and acyl where said acyl is an acid amide or an ester;
or R₆ and R₇, together, are a nitrogen protecting group, are joined in a ring structure that optionally includes an additional heteroatom selected from N and O or are a chemical functional group;
each R₁₄ and R₁₅ is, independently, H, C₁-C₁₀ alkyl, a nitrogen protecting group, or R₁₄ and R₁₅, together, are a nitrogen protecting group;
or R₁₄ and R₁₅ are joined in a ring structure that optionally includes an additional heteroatom selected from N and O;
Z₄ is OX, SX, or N(X)₂;
each X is, independently, H, C₁-C₈ alkyl, C₁-C₈ haloalkyl, C(═NH)N(H)R₁₆, C(═O)N(H)R₁₆ or OC(═O)N(H)R₁₆;
R₁₆ is H or C₁-C₈ alkyl;
Z₁, Z₂ and Z₃ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Z₅ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(R₅)(R₆)OR₅, halo, SR₅ or CN;
each q₁ is, independently, an integer from 1 to 10;
each q₂ is, independently, 0 or 1;
q₃ is 0 or an integer from 1 to 10;
q₄ is an integer from 1 to 10;
q₅ is from 0, 1 or 2; and
provided that when q₃ is 0, q₄ is greater than 1.

24. The method of claim 23 wherein said 2′-substituent group is —O— CH₂CH₂OCH₃, —O(CH₂)₂ON(CH₃)₂, —O—(CH₂)₂—O—(CH₂)₂-N(CH₃)₂, —O—CH₃, —OCH₂CH₂CH₂NH₂, —CH₂—CH═CH₂ or fluoro.

25. An oligomeric compound of the formula 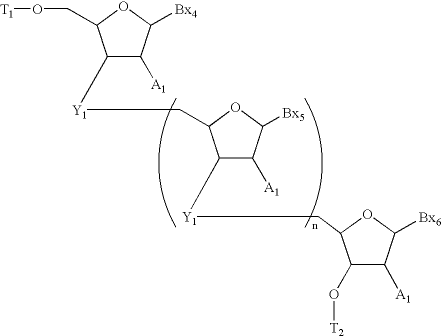
wherein
each Y₁ is, independently, an internucleoside linking group;
each A₁ is, independently, hydrogen or a 2′-substituent group;
T₁ is hydrogen or a hydroxyl protecting group;
T₂ is hydrogen or a hydroxyl protecting group; and
n is from 2 to about 50;
each of Bx₄, Bx₅ and Bx₆ is a heterocyclic base moiety wherein at least one of Bx₄, Bx₅ and Bx₆ is a tricyclic heterocyclic base moiety of the formula; 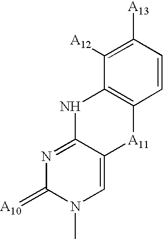
wherein
A₁₀ is S; and A₁₁ is CH₂, O or S; or
A₁₀ is O and A₁₁ is CH₂;
one of A₁₂ and A₁₃ is hydrogen and the other of A₁₂ and A₁₃ is a group of formula;
—O—(CH₂)ₚ₁—G₂
or 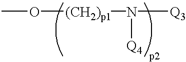
wherein
each A₈ and A₉ is hydrogen or one of A₈ and A₉ is hydrogen and the other of A₈ and A₉ is selected from the group consisting of:
—O—(CH₂)ₚ₁—G₁
and 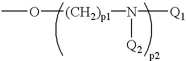
wherein
G₁ is —CN, —OA₂₀, —SA₂₀, —N(H)A₂₀, —ON(H)A₂₀ or —C(═NH)N(H)A₂₀;
Q₁ is H, —NHA₂₀, —C(═O)N(H)A₂₀, —C(═S)N(H)A₂₀ or —C(═NH)N(H)A₂₀,
each Q₂ is, independently, H or Pg;
A₂₀ is H, Pg, substituted or unsubstituted C₁-C₁₀ alkyl, acetyl, benzyl, —(CH₂)ₚ₃NH₂, —(CH₂)ₚ₃N(H)Pg, a D or L α-amino acid, or a peptide derived from D, L or racemic α-amino acids;
Pg is a nitrogen, oxygen or thiol protecting group;
each p1 is, independently, from 2 to about 6;
p2 is from 1 to about 3; and
p3 is from 1 to about 4.

26. The oligomeric compound of claim 25 wherein:
A₁₃ is H;
A₁₂ is —O—(CH₂)₂—N(H)A₂₁, —O—(CH₂)₂—ON(H)A₁₂ or —O—(CH₂)₂— C(═NH)N(H)A₂₁, —O—(CH₂)₃—C(═NH)N(H)A₂₁, —O—(CH₂)₂—C(═O)N(H)A₂₁, —O— (CH₂)₂—C(═S)N(H)A₂₁ or —O—(CH₂)₂—N(H)C(═NH)N(H)A₂₁; and
A₂₁ is hydrogen or an amino protecting group.

27. The oligomeric compound of claim 26 wherein A₁₀ is S.

28. The oligomeric compound of claim 27 wherein A₁₁ is O.

29. The oligomeric compound of claim 25 wherein at least one of Bx₄ and Bx₆ is a tricyclic heterocyclic base moiety.

30. The oligomeric compound of claim 25 wherein each of said internucleoside linking group is a phosphorus-containing internucleoside linking group.

31. The oligomeric compound of claim 30 wherein each of said phosphorus containing internucleoside linking groups is selected from the group consisting of phosphodiester, phosphorothioate, chiral phosphorothioate, phosphorodithioate, phosphotriester, aminoalkylphosphotriester, methyl phosphonate, alkyl phosphonate, 5′-alkylene phosphonate, chiral phosphonate, phosphinate, phosphoramidate, 3′-amino phosphoramidate, aminoalkylphosphoramidate, thionophosphoramidate, thionoalkylphosphonate, thionoalkylphosphotriester, selenophosphate and boranophosphate.

32. The oligomeric compound of claim 25 wherein greater than 90% of said internucleoside linking groups are phosphodiester internucleoside linking groups.

33. The oligomeric compound of claim 25 wherein at least one of said internucleoside linking groups is a non-phosphorus containing internucleoside linking group.

34. The oligomeric compound of claim 33 wherein greater than 90% of said internucleoside linking groups are non-phosphorus containing internucleoside linking groups.

35. The oligomeric compound of claim 34 wherein each of said non-phosphorus containing internucleoside linking groups is, independently, selected from the group consisting of morpholino; siloxane; sulfide; sulfoxide; sulfone; formacetyl; thioformacetyl; methylene formacetyl; thioformacetyl; sulfamate; methyleneimino; methylenehydrazino; sulfonate; sulfonamide; and amide.

36. The oligomeric compound of claim 35 wherein each of said non-phosphorus containing internucleoside linking groups is, independently, selected from the group consisting of CH₂—NH—O—CH₂—, —CH₂—N(CH₃)—O—CH₂—, —CH₂—O—N(CH₃)—CH₂—, —CH₂—N(CH₃)— N(CH₃)—CH₂— and —O—N(CH₃)—CH₂—CH₂—.

37. The oligomeric compound of claim 25 wherein said oligomeric compound is a gapmer, hemimer or inverted gapmer.

38. The oligomeric compound of claim 37 wherein the oligomeric compound comprises at least one 2′-O—CH₂CH₂—O—OH₃ substituent group in at least one region of said gapmer, hemimer or inverted gapmer.

39. The oligomeric compound of claim 25 wherein n is from about 8 to about 30.

40. The oligomeric compound of claim 25 wherein n is from about 15 to about 25.

41. The oligomeric compound of claim 25 wherein each of said Bx₁ and Bx₃ that is not a tricyclic heterocyclic base moiety and each of said Bx₂ is, independently, selected from the group consisting of adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

42. The oligomeric compound of claim 25 wherein each sugar substituent group is, independently, C₁-C₂₀ alkyl, C₂-C₂₀ alkenyl, C₂-C₂₀ alkynyl, C₅-C₂₀ aryl, —O— alkyl —O-alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—R), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—R), sulfone (—S(═O)₂—R), disulfide (—S—S—R), silyl, heterocyclyl, carboxyclyl, an intercalator, a reporter group, a conjugate group, polyamine, polyamide, polyalkylene glycol or a polyether of the formula (—O-alkyl)ₘ, where m is 1 to about 10;
wherein each R is, independently, hydrogen, a protecting group or substituted or unsubstituted alkyl, alkenyl, or alkynyl wherein the substituent groups are selected from haloalkyl, alkenyl, alkoxy, thioalkoxy, haloalkoxy or aryl as well as halogen, hydroxyl, amino, azido, carboxy, cyano, nitro, mercapto, a sulfide group, a sulfonyl group and a sulfoxide group;
or each sugar substituent group has one of formula I or II; 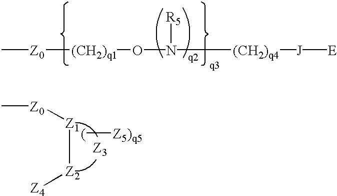
wherein:
Z₀ is O, S or NH;
J is a single bond, O or C(═O);
E is C₁-C₁₀ alkyl, N(R₅)(R₆), N(R₅)(R₇), N═C(R₅ₐ)(R₆ₐ), N═C(R₅ₐ)(R₇ₐ) or has one of formula III or IV; 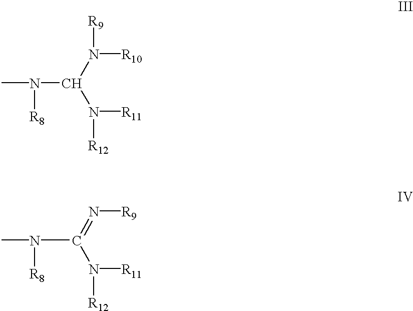
each R₈, R₉, R₁₀, R₁₁ and R₁₂ is, independently, hydrogen, C(O)R₁₃, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, R₉ and R₁₀, together form a phthalimido moiety with the nitrogen atom to which they are attached;
or optionally, R₉ and R₁₀, together form a phthalimido moiety with the nitrogen atom to which they are attached;
each R₁₃ is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
R₅ is hydrogen, a nitrogen protecting group or —T—L,
R₅ₐ is hydrogen, a nitrogen protecting group or —T—L,
T is a bond or a linking moiety;
L is a chemical functional group, a conjugate group or a solid support medium;
each R₆ and R₇ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(R₁₄)(R₁₅), guanidino and acyl where said acyl is an acid amide or an ester;
or R₆ and R₇, together, are a nitrogen protecting group, are joined in a ring structure that optionally includes an additional heteroatom selected from N and O or are a chemical functional group;
each R₁₄ and R₁₅ is, independently, H, C₁-C₁₀ alkyl, a nitrogen protecting group, or R₁₄ and R₁₅, together, are a nitrogen protecting group;
each R₁₄ and R₁₅ are joined in a ring structure that optionally includes an additional heteroatom selected from N and O;
Z₄ is OX, SX, or N(X)₂;
each X is, independently, H, C₁-C₈ alkyl, C₁-C₈ haloalkyl, C(═NH)N(H)R₁₆, C(═O)N(H)R₁₆ or OC(═O)N(H)R₁₆;
R₁₆ is H or C₁-C₈ alkyl;
Z₁, Z₂ and Z₃ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Z₅ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(R₅)(R₆)OR₅, halo, SR₅ or CN;
each q₁ is, independently, an integer from 1 to 10;
each q₂ is, independently, 0 or 1;
q₃ is 0 or an integer from 1 to 10;
q₄ is an integer from 1 to 10;
q₅ is from 0, 1 or 2; and
provided that when q₃ is 0, q₄ is greater than 1.

43. The oligomeric compound of claim 41 wherein said 2′-substituent group is —O—CH₂CH₂OCH₃, —O(CH₂)₂ON(CH₃)₂, —O—(CH₂)₂—O—(CH₂)₂—N(CH₃)₂, —O—CH₃, —OCH₂CH₂CH₂NH₂, —CH₂—CH═CH₂ or fluoro.

44. An oligomeric compound of the formula: 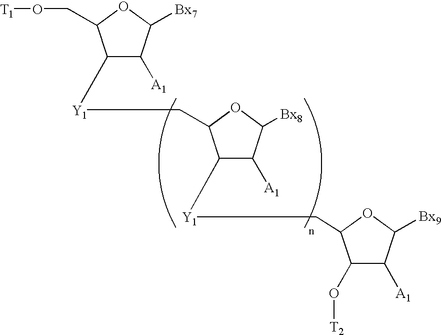
wherein
each Y₁ is, independently, an internucleoside linking group;
each A₁ is, independently, hydrogen or a 2′-substituent group;
T₁ is hydrogen or a hydroxyl protecting group;
T₂ is hydrogen or a hydroxyl protecting group; and
n is from 2 to about 50;
each of Bx₇, Bx₈ and Bx₉ is a heterocyclic base moiety wherein at least one of Bx₇, Bx₈ and Bx₉ is a tricyclic heterocyclic base moiety of the formula; 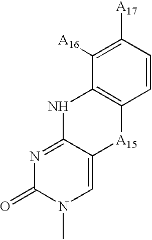
wherein
A₁₅ is O or S;
A₁₆ is H; and
A₁₇ is a group of formula:
—O—(CH₂)ₚ₁—G₁
and 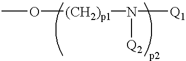
wherein
G₁ is —CN, —OA₂₀, —SA₂₀, —N(H)A₂₀, —ON(H)A₂₀ or —C(═NH)N(H)A₂₀;
Q₁ is H, —NHA₂₀, —C(═O)N(H)A₂₀, —C(═S)N(H)A₂₀ or —C(═NH)N(H)A₂₀,
each Q₂ is, independently, H or Pg;
A₂₀ is H, Pg, substituted or unsubstituted C₁-C₁₀ alkyl, acetyl, benzyl, —(CH₂)ₚ₃NH₂, —(CH₂)ₚ₃N(H)Pg, a D or L α-amino acid, or a peptide derived from D, L or racemic α-amino acids;
Pg is a nitrogen, oxygen or thiol protecting group;
each p1 is, independently, from 2 to about 6;
p2 is from 1 to about 3;
p3 is from 1 to about 4;
or A₁₇ is H and A₁₆ is selected from the group consisting of —O— (CH₂)ₚ₁C(═NH)N(H)A₂₀, —O—(CH₂)ₚ₁N(H)—C(═O)N(H)A₂₀ or —O—(CH₂)ₚ₁N(H)—C(═S)N(H)A₂₀.

45. The oligomeric compound of claim 44 wherein:
A₁₆ is H;
A₁₇ is —O—(CH₂)₂—N(H)A₂₁, —O—(CH₂)₂—ON(H)A₂₁ or —O—(CH₂)₂— C(═NH)N(H)A₂₁, —O—(CH₂)₃—C(50 NH)N(H)A₂₁, —O—(CH₂)₂—C(═O)N(H)A₂₁, —O— (CH₂)₂—C(═S)N(H)A₂₁ or —O—(CH₂)₂—N(H)C(═NH)N(H)A₂₁; and
A₂₁ is hydrogen or an amino protecting group.

46. The oligomeric compound of claim 44 wherein A₁₅ is S.

47. The oligomeric compound of claim 55 wherein A₁₅ is O.

48. The oligomeric compound of claim 44 wherein each of said internucleosides linking groups is a phosphorus-containing internucleoside linking group.

49. The oligomeric compound of claim 48 wherein each of said phosphorus containing internucleoside linking groups is selected from the group consisting of phosphodiester, phosphorothioate, chiral phosphorothioate, phosphorodithioate, phosphotriester, aminoalkylphosphotriester, methyl phosphonate, alkyl phosphonate, 5′-alkylene phosphonate, chiral phosphonate, phosphinate, phosphoramidate, 3′-amino phosphoramidate, aminoalkylphosphoramide, thionophosphoramidate, thionoalkylphosphonate, thionoalkylphosphotriester, selenophosphate and boranophosphate.

50. The oligomeric compound of claim 44 wherein greater than 90% of said internucleoside linking groups are phosphodiester internucleoside linking groups.

51. The oligomeric compound of claim 44 wherein at least one of said internucleoside linking groups is a non-phosphorus containing internucleoside linking group.

52. The oligomeric compound of claim 51 wherein greater than 90% of said internucleoside linking groups are non-phosphorus containing internucleoside linking groups.

53. The oligomeric compound of claim 52 wherein each of said non-phosphorus containing internucleoside linking groups is, independently, selected from the group consisting of morpholino; siloxane; sulfide; sulfoxide; sulfone; formacetyl; thioformacetyl; methylene formacetyl; thioformacetyl; sulfamate; methyleneimino; methylenehydrazino; sulfonate; sulfonamide; and amide.

54. The oligomeric compound of claim 53 wherein each of said non-phosphorus containing internucleoside linking groups is, independently, selected from the group consisting of CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂—.

55. The oligomeric compound of claim 44 wherein said oligomeric compound is a gapmer, hemimer or inverted gapmer.

56. The oligomeric compound of claim 55 wherein the oligomeric compound comprises at least one 2′—O—CH₂CH₂O—CH₃ substituent group in at least one region of said gapmer, hemimer or inverted gapmer.

57. The oligomeric compound of claim 44 wherein n is from about 8 to about 30.

58. The oligomeric compound of claim 44 wherein n is from about 15 to about 25.

59. The oligomeric compound of claim 44 wherein each of said Bx₂, and each of said Bx₁ and Bx₃ that is not a tricyclic heterocyclic base moiety is, independently, selected from the group consisting of adeninyl, guaninyl, thyminyl, cytosinyl, uracilyl, 5-methylcytosinyl (5-me-C), 5-hydroxymethyl cytosinyl, xanthinyl, hyposanthinyl, 2aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

60. The oligomeric compound of claim 44 wherein each sugar substituent group is, independently, C₁-C₂₀ alkyl, C₂-C₂₀ alkenyl, C₂-C₂₀ alkynyl, C₅-C₂₀ aryl, —O-alkyl, —O-alkenyl, —O-alkynyl, —O=alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—R), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—R), sulfone (—S(═O)₂—R), disulfide (—S—S—R), silyl, heterocyclyl, carboxyclyl, an intercalator, a reporter group, a conjugate group, polyamine, polyamide, polyalkylene glycol or a polyether of the formula (—O-alkyl)ₘ, where m is 1 to about 10;
wherein each R is, independently, hydrogen, a protecting group or substituted or unsubstituted alkyl, alkenyl, or alkynyl wherein the substituent groups are selected from haloalkyl, alkenyl, alkoxy, thioalkoxy, haloalkoxy or aryl as well as halogen, hydroxyl, amino, azido, carboxy, cyano, nitro, mercapto, a sulfide group, a sulfonyl group and a sulfoxide group;
or each sugar substituent group has one of formula I or II; 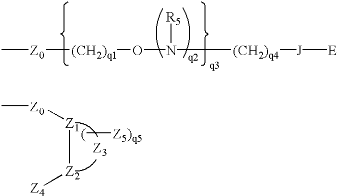
wherein:
Z₀ is O, S or NH;
J is a single bond, O or C(═O);
E is C₁-C₁₀ alkyl, N(R₅)(R₆), N(R₅)(R₇), N═C(R₅ₐ)(R₆ₐ), N═C(R₅ₐ)(R₇ₐ) or has one of formula III or IV; 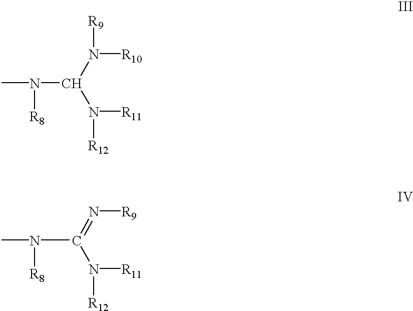
each R₈, R₉, R₁₀, R₁₁ and R₁₂ is, independently, hydrogen C(O)R₁₃, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, R₉ and R₁₀, together form a phthalimido moiety with the nitrogen atom to which they are attached;
or optionally, R₁₁ and R₁₂, together form a phthalimido moiety with the nitrogen atom to which they are attached;
each R₁₃ is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
R₅ is hydrogen, a nitrogen protecting group or —T—L,
R₅ₐ is hydrogen, a nitrogen protecting group or —T—L,
T is a bond or a linking moiety;
L is a chemical functional group, a conjugate group or a solid support medium;
each R₆ and R₇ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(R₁₄)(R₁₅), guanidino and acyl where said acyl is an acid amide or an ester;
or R₆ and R₇, together are a nitrogen protecting group, are joined in a ring structure that optionally includes an additional heteroatom selected from n and O or are a chemical functional group;
each R₁₄ and R₁₅ is, independently, H, C₁-C₁₀ alkyl, a nitrogen protecting group, or R₁₄ and R₁₅, together, are a nitrogen protecting group;
or R₁₄ and R₁₅ are joined in a ring structure that optionally includes an additional heteroatom selected from N and O;
Z₄ is OX, SX, or N(X)₂;
each X is, independently, H, C₁-C₈ alkyl, C₁-C₈ haloalkyl, C(═NH)N(H)R₁₆, C(═O)N(H)R₁₆ or OC(═O)N(H)R₁₆;
R₁₆ is H or C₁-C₈ alkyl;
Z₁, Z₂ and Z₃ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocycle;
Z₅ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(R₅)(R₆)OR₅, halo, SR₅ or CN;
each q₁ is, independently, an integer from 1 to 10;
each q₂ is, independently, 0 or 1;
q₃ is 0 or an integer from 1 to 10;
q₄ is an integer from 1 to 10;
q₅ is from 0, 1 or 2; and
provided that when q₃ is 0, q₄ is greater than 1.

61. The oligomeric compound of claim 60 wherein said 2′-substituent group is —O—CH₂CH₂OCH₃, —O(CH₂)₂ON(CH₃)₂, —O—(CH₂)₂—O—(CH₂)₂—N(CH₃)₂, —O— CH₃, —OCH₂CH₂CH₂NH₂, —CH₂—CH═CH₂ or fluoro.


SEQUENCE LISTING (55 sequences)

SEQ ID NO: 1 - DNA, 20 bp, Artificial Sequence
  Novel Sequence
ngcatcccccaggccaccan

SEQ ID NO: 2 - DNA, 17 bp, Artificial Sequence
  Novel Sequence
nnncgctgtgatgcnnn

SEQ ID NO: 3 - DNA, 20 bp, Artificial Sequence
  Novel Sequence
nnngtcatcgctnnnnnnnn

SEQ ID NO: 4 - DNA, 20 bp, Artificial Sequence
  Novel Sequence
tgcatcccccaggccaccat

SEQ ID NO: 5 - DNA, 20 bp, Artificial Sequence
  Novel Sequence
ngcatcccccaggccaccan

SEQ ID NO: 6 - DNA, 17 bp, Artificial Sequence
  Novel Sequence
tcccgctgtgatgcatt

SEQ ID NO: 7 - DNA, 17 bp, Artificial Sequence
  Novel Sequence
nnncgctgtgatgcnnn

SEQ ID NO: 8 - DNA, 20 bp, Artificial Sequence
  Novel Sequence
nnnnnttccacactcnnnnn

SEQ ID NO: 9 - DNA, 20 bp, Artificial Sequence
  Novel Sequence
nnnnnttccacactcnnnnn

SEQ ID NO: 10 - DNA, 20 bp, Artificial Sequence
  Novel Sequence
nnnnnttccacactcnnnnn

SEQ ID NO: 11 - DNA, 20 bp, Artificial Sequence
  Novel Sequence
ccggtacccnnnnntnnnnn

SEQ ID NO: 12 - DNA, 20 bp, Artificial Sequence
  Novel Sequence
ncggtacccnnnnntnnnnn

SEQ ID NO: 13 - DNA, 20 bp, Artificial Sequence
  Novel Sequence
ctagattcnnnnctctcgtn

SEQ ID NO: 14 - DNA, 20 bp, Artificial Sequence
  Novel Sequence
ntagattcnnnnctctcgtc

SEQ ID NO: 15 - DNA, 20 bp, Artificial Sequence
  Novel Sequence
ntagattcnnnnctctcgtn

SEQ ID NO: 16 - DNA, 20 bp, Artificial Sequence
  Novel Sequence
nnnnnttccacactcnnnnn

SEQ ID NO: 17 - DNA, 20 bp, Artificial Sequence
  Novel Sequence
ccggtacccnnnnntnnnnn

SEQ ID NO: 18 - DNA, 20 bp, Artificial Sequence
  Novel Sequence
nnnnnttccacactcnnnnn

SEQ ID NO: 19 - DNA, 20 bp, Artificial Sequence
  Novel Sequence
ccggtacccnnnnnnnnnnn

SEQ ID NO: 20 - DNA, 20 bp, Artificial Sequence
  Novel Sequence
nnnnnttccacactcnnnnn

SEQ ID NO: 21 - DNA, 20 bp, Artificial Sequence
  Novel Sequence
nnnnnttccacactcnnnnn

SEQ ID NO: 22 - DNA, 20 bp, Artificial Sequence
  Novel Sequence
nnnnnttccacactcnnnnn

SEQ ID NO: 23 - DNA, 21 bp, Artificial Sequence
  Novel Sequence
nnnnnnttccacactcnnnnn

SEQ ID NO: 24 - DNA, 21 bp, Artificial Sequence
  Novel Sequence
ccggtacccnnnnnnnnnnan

SEQ ID NO: 25 - DNA, 22 bp, Artificial Sequence
  Novel Sequence
nccggtacccnnnnnnnnnnan

SEQ ID NO: 26 - DNA, 20 bp, Artificial Sequence
  Novel Sequence
nnnnnttccacactcnnnnn

SEQ ID NO: 27 - DNA, 20 bp, Artificial Sequence
  Novel Sequence
nnnnnttccacactcnnnnn

SEQ ID NO: 28 - DNA, 20 bp, Artificial Sequence
  Novel Sequence
nnnnnttccacactcnnnnn

SEQ ID NO: 29 - DNA, 20 bp, Artificial Sequence
  Novel Sequence
nnnnnttccacactcnnnnn

SEQ ID NO: 30 - DNA, 20 bp, Artificial Sequence
  Novel Sequence
nnnnnttccacactcnnnnn

SEQ ID NO: 31 - DNA, 20 bp, Artificial Sequence
  Novel Sequence
nnnnnttccacactcnnnnn

SEQ ID NO: 32 - DNA, 20 bp, Artificial Sequence
  Novel Sequence
nnnnnttccacactcnnnnn

SEQ ID NO: 33 - DNA, 20 bp, Artificial Sequence
  Novel Sequence
nnnnnttccacactcnnnnn

SEQ ID NO: 34 - DNA, 10 bp, Artificial Sequence
  Novel Sequence
ttttnttttt

SEQ ID NO: 35 - DNA, 10 bp, Artificial Sequence
  Novel Sequence
tctcnctctc

SEQ ID NO: 36 - DNA, 10 bp, Artificial Sequence
  Novel Sequence
tctcnctctc

SEQ ID NO: 37 - DNA, 18 bp, Artificial Sequence
  Novel Sequence
ctcgtaccntcccggtcc

SEQ ID NO: 38 - DNA, 10 bp, Artificial Sequence
  Novel Sequence
gngtanacgc

SEQ ID NO: 39 - DNA, 10 bp, Artificial Sequence
  Novel Sequence
gcgtanangc

SEQ ID NO: 40 - DNA, 15 bp, Artificial Sequence
  Novel Sequence
aaaaagagagggaga

SEQ ID NO: 41 - DNA, 10 bp, Artificial Sequence
  Novel Sequence
gngtanacgc

SEQ ID NO: 42 - DNA, 20 bp, Artificial Sequence
  Novel Sequence
atgcattctgcccccaagga

SEQ ID NO: 43 - DNA, 20 bp, Artificial Sequence
  Novel Sequence
atgnattctgcccccaagga

SEQ ID NO: 44 - DNA, 20 bp, Artificial Sequence
  Novel Sequence
atgcattntgcccccaagga

SEQ ID NO: 45 - DNA, 20 bp, Artificial Sequence
  Novel Sequence
atgcattctgnccccaagga

SEQ ID NO: 46 - DNA, 20 bp, Artificial Sequence
  Novel Sequence
atgcattctgcncccaagga

SEQ ID NO: 47 - DNA, 20 bp, Artificial Sequence
  Novel Sequence
atgcattctgccnccaagga

SEQ ID NO: 48 - DNA, 20 bp, Artificial Sequence
  Novel Sequence
atgcattctgcccncaagga

SEQ ID NO: 49 - DNA, 20 bp, Artificial Sequence
  Novel Sequence
atgcattctgccccnaagga

SEQ ID NO: 50 - DNA, 22 bp, Artificial Sequence
  Novel Sequence
ctagattccacactctctcgtc

SEQ ID NO: 51 - DNA, 20 bp, Artificial Sequence
  Novel Sequence
ntagattccacactctcgtc

SEQ ID NO: 52 - DNA, 20 bp, Artificial Sequence
  Novel Sequence
ctagattccacactctcgtn

SEQ ID NO: 53 - DNA, 20 bp, Artificial Sequence
  Novel Sequence
ntagattccacactctcgtn

SEQ ID NO: 54 - DNA, 19 bp, Artificial Sequence
  Novel Sequence
ttttttttttttttttttn

SEQ ID NO: 55 - DNA, 19 bp, Artificial Sequence
  Novel Sequence
ttttttttttttttttttn